SEQUENCE LISTING (414 sequences)

SEQ ID NO: 1 - RNA, 36 nt, Artificial Sequence
  Synthetic Construct
guugcaaaacccaagaaauccgucuuucauugacgg

SEQ ID NO: 2 - RNA, 36 nt, Artificial Sequence
  Synthetic Construct
aauagcggcccuaagaaauccgucuuucauugacgg

SEQ ID NO: 3 - RNA, 36 nt, Artificial Sequence
  Synthetic Construct
auuggaacuggcgagaaauccgucuuucauugacgg

SEQ ID NO: 4 - RNA, 36 nt, Artificial Sequence
  Synthetic Construct
ccagcaacaccuaagaaauccgucuuucauugacgg

SEQ ID NO: 5 - RNA, 36 nt, Artificial Sequence
  Synthetic Construct
cggcgcucgaauaggaaauccgucuuucauugacgg

SEQ ID NO: 6 - RNA, 36 nt, Artificial Sequence
  Synthetic Construct
guggcaacaccuaagaaauccgucuuucauugacgg

SEQ ID NO: 7 - RNA, 36 nt, Artificial Sequence
  Synthetic Construct
guugcaacaccuaagaaauccgucuuucauugacgg

SEQ ID NO: 8 - RNA, 36 nt, Artificial Sequence
  Synthetic Construct
guugcaaugccuaagaaauccgucuuucauugacgg

SEQ ID NO: 9 - RNA, 34 nt, Artificial Sequence
  Synthetic Construct
gcaacaccuaagaaauccgucuuucauugacggg

SEQ ID NO: 10 - RNA, 23 nt, Artificial Sequence
  Synthetic Construct
agaaauccgucuuucauugacgg

SEQ ID NO: 11 - DNA, 30 bp, Artificial Sequence
  Synthetic Construct
ctttgtgacacatttgtttgagaatcaaaa

SEQ ID NO: 12 - DNA, 30 bp, Artificial Sequence
  Synthetic Construct
accgattttgattctcaaacaaatgtgtca

SEQ ID NO: 13 - DNA, 30 bp, Artificial Sequence
  Synthetic Construct
tgattctcaaacaaatgtgtcacaaagtaa

SEQ ID NO: 14 - DNA, 30 bp, Artificial Sequence
  Synthetic Construct
gattctcaaacaaatgtgtcacaaagtaag

SEQ ID NO: 15 - DNA, 30 bp, Artificial Sequence
  Synthetic Construct
attctcaaacaaatgtgtcacaaagtaagg

SEQ ID NO: 16 - DNA, 30 bp, Artificial Sequence
  Synthetic Construct
tcaaacaaatgtgtcacaaagtaaggattc

SEQ ID NO: 17 - DNA, 30 bp, Artificial Sequence
  Synthetic Construct
tgatgtgtatatcacagacaaaactgtgct

SEQ ID NO: 18 - DNA, 30 bp, Artificial Sequence
  Synthetic Construct
aagagcaacagtgctgtggcctggagcaac

SEQ ID NO: 19 - DNA, 30 bp, Artificial Sequence
  Synthetic Construct
gcatgtgcaaacgccttcaacaacagcatt

SEQ ID NO: 20 - DNA, 30 bp, Artificial Sequence
  Synthetic Construct
catgtgcaaacgccttcaacaacagcatta

SEQ ID NO: 21 - DNA, 30 bp, Artificial Sequence
  Synthetic Construct
aacaacagcattattccagaagacaccttc

SEQ ID NO: 22 - DNA, 30 bp, Artificial Sequence
  Synthetic Construct
ttccagaagacaccttcttccccagcccag

SEQ ID NO: 23 - DNA, 30 bp, Artificial Sequence
  Synthetic Construct
cagaagacaccttcttccccagcccaggta

SEQ ID NO: 24 - DNA, 30 bp, Artificial Sequence
  Synthetic Construct
ttccccagcccaggtaagggcagctttggt

SEQ ID NO: 25 - DNA, 30 bp, Artificial Sequence
  Synthetic Construct
cccagcccaggtaagggcagctttggtgcc

SEQ ID NO: 26 - DNA, 30 bp, Artificial Sequence
  Synthetic Construct
ggtgccttcgcaggctgtttccttgcttca

SEQ ID NO: 27 - DNA, 30 bp, Artificial Sequence
  Synthetic Construct
gtgccttcgcaggctgtttccttgcttcag

SEQ ID NO: 28 - DNA, 30 bp, Artificial Sequence
  Synthetic Construct
gcaggctgtttccttgcttcaggaatggcc

SEQ ID NO: 29 - DNA, 30 bp, Artificial Sequence
  Synthetic Construct
ctgaagcaaggaaacagcctgcgaaggcac

SEQ ID NO: 30 - DNA, 30 bp, Artificial Sequence
  Synthetic Construct
cctgggctggggaagaaggtgtcttctgga

SEQ ID NO: 31 - DNA, 30 bp, Artificial Sequence
  Synthetic Construct
tcccacagatatccagaaccctgaccctgc

SEQ ID NO: 32 - DNA, 30 bp, Artificial Sequence
  Synthetic Construct
tggaataatgctgttgttgaaggcgtttgc

SEQ ID NO: 33 - DNA, 30 bp, Artificial Sequence
  Synthetic Construct
ttgaaggcgtttgcacatgcaaagtcagat

SEQ ID NO: 34 - DNA, 30 bp, Artificial Sequence
  Synthetic Construct
gcacatgcaaagtcagatttgttgctccag

SEQ ID NO: 35 - DNA, 30 bp, Artificial Sequence
  Synthetic Construct
cacatgcaaagtcagatttgttgctccagg

SEQ ID NO: 36 - DNA, 30 bp, Artificial Sequence
  Synthetic Construct
gttgctccaggccacagcactgttgctctt

SEQ ID NO: 37 - DNA, 30 bp, Artificial Sequence
  Synthetic Construct
ttgctccaggccacagcactgttgctcttg

SEQ ID NO: 38 - DNA, 30 bp, Artificial Sequence
  Synthetic Construct
ctccaggccacagcactgttgctcttgaag

SEQ ID NO: 39 - DNA, 30 bp, Artificial Sequence
  Synthetic Construct
ctcttgaagtccatagacctcatgtctagc

SEQ ID NO: 40 - DNA, 30 bp, Artificial Sequence
  Synthetic Construct
aagtccatagacctcatgtctagcacagtt

SEQ ID NO: 41 - DNA, 30 bp, Artificial Sequence
  Synthetic Construct
tgtctgtgatatacacatcagaatccttac

SEQ ID NO: 42 - DNA, 30 bp, Artificial Sequence
  Synthetic Construct
gtctgtgatatacacatcagaatccttact

SEQ ID NO: 43 - DNA, 30 bp, Artificial Sequence
  Synthetic Construct
tctgtgatatacacatcagaatccttactt

SEQ ID NO: 44 - DNA, 30 bp, Artificial Sequence
  Synthetic Construct
gtgacacatttgtttgagaatcaaaatcgg

SEQ ID NO: 45 - DNA, 30 bp, Artificial Sequence
  Synthetic Construct
aaggcgtttgcacatgcaaagtcagatttg

SEQ ID NO: 46 - DNA, 30 bp, Artificial Sequence
  Synthetic Construct
tgacacatttgtttgagaatcaaaatcggt

SEQ ID NO: 47 - DNA, 30 bp, Artificial Sequence
  Synthetic Construct
gtttgagaatcaaaatcggtgaataggcag

SEQ ID NO: 48 - DNA, 30 bp, Artificial Sequence
  Synthetic Construct
tttgagaatcaaaatcggtgaataggcaga

SEQ ID NO: 49 - DNA, 30 bp, Artificial Sequence
  Synthetic Construct
gagaatcaaaatcggtgaataggcagacag

SEQ ID NO: 50 - DNA, 30 bp, Artificial Sequence
  Synthetic Construct
agaatcaaaatcggtgaataggcagacaga

SEQ ID NO: 51 - DNA, 30 bp, Artificial Sequence
  Synthetic Construct
tcactggatttagagtctctcagctggtac

SEQ ID NO: 52 - DNA, 30 bp, Artificial Sequence
  Synthetic Construct
agagtctctcagctggtacacggcagggtc

SEQ ID NO: 53 - DNA, 30 bp, Artificial Sequence
  Synthetic Construct
gagtctctcagctggtacacggcagggtca

SEQ ID NO: 54 - DNA, 30 bp, Artificial Sequence
  Synthetic Construct
tggatatctgtgggacaagaggatcagggt

SEQ ID NO: 55 - DNA, 30 bp, Artificial Sequence
  Synthetic Construct
ggacatgatctcatttccctctttgcccca

SEQ ID NO: 56 - DNA, 30 bp, Artificial Sequence
  Synthetic Construct
agcatccagagcaggctgactctcccaccc

SEQ ID NO: 57 - DNA, 30 bp, Artificial Sequence
  Synthetic Construct
ttccttttagaaagttcctgtgatgtcaag

SEQ ID NO: 58 - DNA, 30 bp, Artificial Sequence
  Synthetic Construct
cagcatccagagcaggctgactctcccacc

SEQ ID NO: 59 - DNA, 30 bp, Artificial Sequence
  Synthetic Construct
taaaaggaaaaacagacattctttcagcat

SEQ ID NO: 60 - DNA, 30 bp, Artificial Sequence
  Synthetic Construct
tccttttagaaagttcctgtgatgtcaagc

SEQ ID NO: 61 - DNA, 30 bp, Artificial Sequence
  Synthetic Construct
ccttttagaaagttcctgtgatgtcaagct

SEQ ID NO: 62 - DNA, 30 bp, Artificial Sequence
  Synthetic Construct
cttttagaaagttcctgtgatgtcaagctg

SEQ ID NO: 63 - DNA, 30 bp, Artificial Sequence
  Synthetic Construct
tagaaagttcctgtgatgtcaagctggtcg

SEQ ID NO: 64 - DNA, 30 bp, Artificial Sequence
  Synthetic Construct
agaaagttcctgtgatgtcaagctggtcga

SEQ ID NO: 65 - DNA, 30 bp, Artificial Sequence
  Synthetic Construct
gaaagttcctgtgatgtcaagctggtcgag

SEQ ID NO: 66 - DNA, 30 bp, Artificial Sequence
  Synthetic Construct
ctgtgatgtcaagctggtcgagaaaagctt

SEQ ID NO: 67 - DNA, 30 bp, Artificial Sequence
  Synthetic Construct
gaaacaggtaagacaggggtctagcctggg

SEQ ID NO: 68 - DNA, 30 bp, Artificial Sequence
  Synthetic Construct
aaacaggtaagacaggggtctagcctgggt

SEQ ID NO: 69 - DNA, 30 bp, Artificial Sequence
  Synthetic Construct
gcacaggattgcggaagtgatgaacccgca

SEQ ID NO: 70 - DNA, 30 bp, Artificial Sequence
  Synthetic Construct
cgcaatcctgtgcaaacccaggctagaccc

SEQ ID NO: 71 - DNA, 30 bp, Artificial Sequence
  Synthetic Construct
cctgtttcaaagcttttctcgaccagcttg

SEQ ID NO: 72 - DNA, 30 bp, Artificial Sequence
  Synthetic Construct
caaagcttttctcgaccagcttgacatcac

SEQ ID NO: 73 - DNA, 30 bp, Artificial Sequence
  Synthetic Construct
aaagcttttctcgaccagcttgacatcaca

SEQ ID NO: 74 - DNA, 30 bp, Artificial Sequence
  Synthetic Construct
tctcgaccagcttgacatcacaggaacttt

SEQ ID NO: 75 - DNA, 30 bp, Artificial Sequence
  Synthetic Construct
ctcgaccagcttgacatcacaggaactttc

SEQ ID NO: 76 - DNA, 30 bp, Artificial Sequence
  Synthetic Construct
tcgaccagcttgacatcacaggaactttct

SEQ ID NO: 77 - DNA, 30 bp, Artificial Sequence
  Synthetic Construct
acatcacaggaactttctaaaaggaaaaac

SEQ ID NO: 78 - DNA, 30 bp, Artificial Sequence
  Synthetic Construct
ctaaaaggaaaaacagacattctttcagca

SEQ ID NO: 79 - DNA, 30 bp, Artificial Sequence
  Synthetic Construct
tttcagcatccagagcaggctgactctccc

SEQ ID NO: 80 - DNA, 30 bp, Artificial Sequence
  Synthetic Construct
cacaggattgcggaagtgatgaacccgcaa

SEQ ID NO: 81 - DNA, 30 bp, Artificial Sequence
  Synthetic Construct
gttttacagatacgaacctaaactttcaaa

SEQ ID NO: 82 - DNA, 30 bp, Artificial Sequence
  Synthetic Construct
gtatctgtaaaaccaagaggccacagcggt

SEQ ID NO: 83 - DNA, 30 bp, Artificial Sequence
  Synthetic Construct
tgggcttgcatgtcccaggcctgcatggcc

SEQ ID NO: 84 - DNA, 30 bp, Artificial Sequence
  Synthetic Construct
tacagatacgaacctaaactttcaaaacct

SEQ ID NO: 85 - DNA, 30 bp, Artificial Sequence
  Synthetic Construct
acagatacgaacctaaactttcaaaacctg

SEQ ID NO: 86 - DNA, 30 bp, Artificial Sequence
  Synthetic Construct
cagatacgaacctaaactttcaaaacctgt

SEQ ID NO: 87 - DNA, 30 bp, Artificial Sequence
  Synthetic Construct
aaaacctgtcagtgattgggttccgaatcc

SEQ ID NO: 88 - DNA, 30 bp, Artificial Sequence
  Synthetic Construct
ggttccgaatcctcctcctgaaagtggccg

SEQ ID NO: 89 - DNA, 30 bp, Artificial Sequence
  Synthetic Construct
cgaatcctcctcctgaaagtggccgggttt

SEQ ID NO: 90 - DNA, 30 bp, Artificial Sequence
  Synthetic Construct
aatctgctcatgacgctgcggctgtggtcc

SEQ ID NO: 91 - DNA, 30 bp, Artificial Sequence
  Synthetic Construct
atctgctcatgacgctgcggctgtggtcca

SEQ ID NO: 92 - DNA, 30 bp, Artificial Sequence
  Synthetic Construct
aagctgggagtggggtttagggacgcgggt

SEQ ID NO: 93 - DNA, 30 bp, Artificial Sequence
  Synthetic Construct
caaaacctgtcagtgattgggttccgaatc

SEQ ID NO: 94 - DNA, 30 bp, Artificial Sequence
  Synthetic Construct
aacccggccactttcaggaggaggattcgg

SEQ ID NO: 95 - DNA, 30 bp, Artificial Sequence
  Synthetic Construct
caggaggaggattcggaacccaatcactga

SEQ ID NO: 96 - DNA, 30 bp, Artificial Sequence
  Synthetic Construct
aggaggaggattcggaacccaatcactgac

SEQ ID NO: 97 - DNA, 30 bp, Artificial Sequence
  Synthetic Construct
ggaacccaatcactgacaggttttgaaagt

SEQ ID NO: 98 - DNA, 30 bp, Artificial Sequence
  Synthetic Construct
tgaaagtttaggttcgtatctgtaaaacca

SEQ ID NO: 99 - DNA, 30 bp, Artificial Sequence
  Synthetic Construct
gaaagtttaggttcgtatctgtaaaaccaa

SEQ ID NO: 100 - DNA, 30 bp, Artificial Sequence
  Synthetic Construct
aaagtttaggttcgtatctgtaaaaccaag

SEQ ID NO: 101 - DNA, 30 bp, Artificial Sequence
  Synthetic Construct
aggttcgtatctgtaaaaccaagaggccac

SEQ ID NO: 102 - DNA, 30 bp, Artificial Sequence
  Synthetic Construct
aaggcccctcacctcagctggaccacagcc

SEQ ID NO: 103 - DNA, 30 bp, Artificial Sequence
  Synthetic Construct
ggttcgtatctgtaaaaccaagaggccaca

SEQ ID NO: 104 - DNA, 30 bp, Artificial Sequence
  Synthetic Construct
tcaagacgtggggggaaattatctcattat

SEQ ID NO: 105 - DNA, 30 bp, Artificial Sequence
  Synthetic Construct
ctttatgaacactattaaaaaaaaataaac

SEQ ID NO: 106 - DNA, 30 bp, Artificial Sequence
  Synthetic Construct
agaagaagaatactatgtatttctttatga

SEQ ID NO: 107 - DNA, 30 bp, Artificial Sequence
  Synthetic Construct
cccccacgtcttgagaagaagaatactatg

SEQ ID NO: 108 - DNA, 30 bp, Artificial Sequence
  Synthetic Construct
ccccccacgtcttgagaagaagaatactat

SEQ ID NO: 109 - DNA, 30 bp, Artificial Sequence
  Synthetic Construct
atgaaggcatcggcagcaggacatacaaca

SEQ ID NO: 110 - DNA, 30 bp, Artificial Sequence
  Synthetic Construct
tatgtcctgctgccgatgccttcattaaaa

SEQ ID NO: 111 - DNA, 30 bp, Artificial Sequence
  Synthetic Construct
taatgaaggcatcggcagcaggacatacaa

SEQ ID NO: 112 - DNA, 30 bp, Artificial Sequence
  Synthetic Construct
caaatcattttaatgaaggcatcggcagca

SEQ ID NO: 113 - DNA, 24 bp, Artificial Sequence
  Synthetic Construct
attaaaatgatttggaagagcaga

SEQ ID NO: 114 - DNA, 30 bp, Artificial Sequence
  Synthetic Construct
tcgaggccctgctatgctgtgtatctgggc

SEQ ID NO: 115 - DNA, 30 bp, Artificial Sequence
  Synthetic Construct
tctcattatcgaggccctgctatgctgtgt

SEQ ID NO: 116 - DNA, 30 bp, Artificial Sequence
  Synthetic Construct
tttatgaacactattaaaaaaaaataaacc

SEQ ID NO: 117 - DNA, 30 bp, Artificial Sequence
  Synthetic Construct
aatgaaggcatcggcagcaggacatacaac

SEQ ID NO: 118 - DNA, 30 bp, Artificial Sequence
  Synthetic Construct
atgaacactattaaaaaaaaataaacccct

SEQ ID NO: 119 - DNA, 30 bp, Artificial Sequence
  Synthetic Construct
ccgggggggcacagaggtggtagcagcttt

SEQ ID NO: 120 - DNA, 30 bp, Artificial Sequence
  Synthetic Construct
aaaaaaaataaacccctcacaacattctgc

SEQ ID NO: 121 - DNA, 30 bp, Artificial Sequence
  Synthetic Construct
gagagggagaagaggggcaatgcagaggaa

SEQ ID NO: 122 - DNA, 30 bp, Artificial Sequence
  Synthetic Construct
ggagagggagaagaggggcaatgcagagga

SEQ ID NO: 123 - DNA, 30 bp, Artificial Sequence
  Synthetic Construct
cctctgtttggagagggagaagaggggcaa

SEQ ID NO: 124 - DNA, 30 bp, Artificial Sequence
  Synthetic Construct
ggggtaggagagttccctctgtttggagag

SEQ ID NO: 125 - DNA, 30 bp, Artificial Sequence
  Synthetic Construct
acctccttgggggtaggagagttccctctg

SEQ ID NO: 126 - DNA, 30 bp, Artificial Sequence
  Synthetic Construct
cacctccttgggggtaggagagttccctct

SEQ ID NO: 127 - DNA, 30 bp, Artificial Sequence
  Synthetic Construct
ttctcaagacgtggggggaaattatctcat

SEQ ID NO: 128 - DNA, 30 bp, Artificial Sequence
  Synthetic Construct
gtggcattgccgggggggcacagaggtggt

SEQ ID NO: 129 - DNA, 30 bp, Artificial Sequence
  Synthetic Construct
gggtaggatccagttggtggcattgccggg

SEQ ID NO: 130 - DNA, 30 bp, Artificial Sequence
  Synthetic Construct
atcataaattcgggtaggatccagttggtg

SEQ ID NO: 131 - DNA, 30 bp, Artificial Sequence
  Synthetic Construct
agcaatcttaatcataaattcgggtaggat

SEQ ID NO: 132 - DNA, 30 bp, Artificial Sequence
  Synthetic Construct
gcagctcttcagcaatcttaatcataaatt

SEQ ID NO: 133 - DNA, 30 bp, Artificial Sequence
  Synthetic Construct
ggcagctcttcagcaatcttaatcataaat

SEQ ID NO: 134 - DNA, 30 bp, Artificial Sequence
  Synthetic Construct
cctctgccgtgaatgtcaggcagtgacaag

SEQ ID NO: 135 - DNA, 30 bp, Artificial Sequence
  Synthetic Construct
tgcaggacctagagcccaagagaacccact

SEQ ID NO: 136 - DNA, 30 bp, Artificial Sequence
  Synthetic Construct
tgaacactattaaaaaaaaataaacccctc

SEQ ID NO: 137 - DNA, 30 bp, Artificial Sequence
  Synthetic Construct
ttcttctcaagacgtggggggaaattatct

SEQ ID NO: 138 - DNA, 30 bp, Artificial Sequence
  Synthetic Construct
atttttttttaatagtgttcataaagaaat

SEQ ID NO: 139 - DNA, 30 bp, Artificial Sequence
  Synthetic Construct
atagtgttcataaagaaatacatagtattc

SEQ ID NO: 140 - DNA, 30 bp, Artificial Sequence
  Synthetic Construct
ctgaagagctgccaaacactgctgccaccc

SEQ ID NO: 141 - DNA, 30 bp, Artificial Sequence
  Synthetic Construct
agattgctgaagagctgccaaacactgctg

SEQ ID NO: 142 - DNA, 30 bp, Artificial Sequence
  Synthetic Construct
tgattaagattgctgaagagctgccaaaca

SEQ ID NO: 143 - DNA, 30 bp, Artificial Sequence
  Synthetic Construct
atgattaagattgctgaagagctgccaaac

SEQ ID NO: 144 - DNA, 30 bp, Artificial Sequence
  Synthetic Construct
tccctctccaaacagagggaactctcctac

SEQ ID NO: 145 - DNA, 30 bp, Artificial Sequence
  Synthetic Construct
cccctcttctccctctccaaacagagggaa

SEQ ID NO: 146 - DNA, 30 bp, Artificial Sequence
  Synthetic Construct
ccttattgctgcttgtcactgcctgacatt

SEQ ID NO: 147 - DNA, 30 bp, Artificial Sequence
  Synthetic Construct
ctctgcattgcccctcttctccctctccaa

SEQ ID NO: 148 - DNA, 30 bp, Artificial Sequence
  Synthetic Construct
cagatctgcaagattgtaagacagcctgtg

SEQ ID NO: 149 - DNA, 30 bp, Artificial Sequence
  Synthetic Construct
ctcattccagatctgcaagattgtaagaca

SEQ ID NO: 150 - DNA, 30 bp, Artificial Sequence
  Synthetic Construct
ttcctcattccagatctgcaagattgtaag

SEQ ID NO: 151 - DNA, 30 bp, Artificial Sequence
  Synthetic Construct
tgttcttcctcattccagatctgcaagatt

SEQ ID NO: 152 - DNA, 30 bp, Artificial Sequence
  Synthetic Construct
ctcagtgtccagcctgacttctgttcttcc

SEQ ID NO: 153 - DNA, 30 bp, Artificial Sequence
  Synthetic Construct
caatcttgcagatctggaatgaggaagaac

SEQ ID NO: 154 - DNA, 30 bp, Artificial Sequence
  Synthetic Construct
taagacagcctgtgctccctcgctccttcc

SEQ ID NO: 155 - DNA, 30 bp, Artificial Sequence
  Synthetic Construct
ataaagaaatacatagtattcttcttctca

SEQ ID NO: 156 - DNA, 30 bp, Artificial Sequence
  Synthetic Construct
ttgctgcttgtcactgcctgacattcacgg

SEQ ID NO: 157 - DNA, 30 bp, Artificial Sequence
  Synthetic Construct
tcactgcctgacattcacggcagaggcaag

SEQ ID NO: 158 - DNA, 30 bp, Artificial Sequence
  Synthetic Construct
aatagtgttcataaagaaatacatagtatt

SEQ ID NO: 159 - DNA, 30 bp, Artificial Sequence
  Synthetic Construct
taatagtgttcataaagaaatacatagtat

SEQ ID NO: 160 - DNA, 30 bp, Artificial Sequence
  Synthetic Construct
ttaatagtgttcataaagaaatacatagta

SEQ ID NO: 161 - DNA, 30 bp, Artificial Sequence
  Synthetic Construct
tttaatagtgttcataaagaaatacatagt

SEQ ID NO: 162 - DNA, 30 bp, Artificial Sequence
  Synthetic Construct
ttttaatagtgttcataaagaaatacatag

SEQ ID NO: 163 - DNA, 30 bp, Artificial Sequence
  Synthetic Construct
tttttaatagtgttcataaagaaatacata

SEQ ID NO: 164 - DNA, 30 bp, Artificial Sequence
  Synthetic Construct
ctgcttgtcactgcctgacattcacggcag

SEQ ID NO: 165 - DNA, 30 bp, Artificial Sequence
  Synthetic Construct
ttttttaatagtgttcataaagaaatacat

SEQ ID NO: 166 - DNA, 30 bp, Artificial Sequence
  Synthetic Construct
tgaggggtttatttttttttaatagtgttc

SEQ ID NO: 167 - DNA, 30 bp, Artificial Sequence
  Synthetic Construct
ggctctaggtcctgcagaatgttgtgaggg

SEQ ID NO: 168 - DNA, 30 bp, Artificial Sequence
  Synthetic Construct
tcttgggctctaggtcctgcagaatgttgt

SEQ ID NO: 169 - DNA, 30 bp, Artificial Sequence
  Synthetic Construct
agtgggttctcttgggctctaggtcctgca

SEQ ID NO: 170 - DNA, 30 bp, Artificial Sequence
  Synthetic Construct
cctcctgctccccagagactgcctccgcca

SEQ ID NO: 171 - DNA, 30 bp, Artificial Sequence
  Synthetic Construct
acggcagaggcaaggctgctgcagcctccc

SEQ ID NO: 172 - DNA, 30 bp, Artificial Sequence
  Synthetic Construct
tttttttttaatagtgttcataaagaaata

SEQ ID NO: 173 - DNA, 30 bp, Artificial Sequence
  Synthetic Construct
cagatctggaatgaggaagaacagaagtca

SEQ ID NO: 174 - RNA, 30 nt, Artificial Sequence
  Synthetic Construct
cuuugugacacauuuguuugagaaucaaaa

SEQ ID NO: 175 - RNA, 30 nt, Artificial Sequence
  Synthetic Construct
accgauuuugauucucaaacaaauguguca

SEQ ID NO: 176 - RNA, 30 nt, Artificial Sequence
  Synthetic Construct
ugauucucaaacaaaugugucacaaaguaa

SEQ ID NO: 177 - RNA, 30 nt, Artificial Sequence
  Synthetic Construct
gauucucaaacaaaugugucacaaaguaag

SEQ ID NO: 178 - RNA, 30 nt, Artificial Sequence
  Synthetic Construct
auucucaaacaaaugugucacaaaguaagg

SEQ ID NO: 179 - RNA, 30 nt, Artificial Sequence
  Synthetic Construct
ucaaacaaaugugucacaaaguaaggauuc

SEQ ID NO: 180 - RNA, 30 nt, Artificial Sequence
  Synthetic Construct
ugauguguauaucacagacaaaacugugcu

SEQ ID NO: 181 - RNA, 30 nt, Artificial Sequence
  Synthetic Construct
aagagcaacagugcuguggccuggagcaac

SEQ ID NO: 182 - RNA, 30 nt, Artificial Sequence
  Synthetic Construct
gcaugugcaaacgccuucaacaacagcauu

SEQ ID NO: 183 - RNA, 30 nt, Artificial Sequence
  Synthetic Construct
caugugcaaacgccuucaacaacagcauua

SEQ ID NO: 184 - RNA, 30 nt, Artificial Sequence
  Synthetic Construct
aacaacagcauuauuccagaagacaccuuc

SEQ ID NO: 185 - RNA, 30 nt, Artificial Sequence
  Synthetic Construct
uuccagaagacaccuucuuccccagcccag

SEQ ID NO: 186 - RNA, 30 nt, Artificial Sequence
  Synthetic Construct
cagaagacaccuucuuccccagcccaggua

SEQ ID NO: 187 - RNA, 30 nt, Artificial Sequence
  Synthetic Construct
uuccccagcccagguaagggcagcuuuggu

SEQ ID NO: 188 - RNA, 30 nt, Artificial Sequence
  Synthetic Construct
cccagcccagguaagggcagcuuuggugcc

SEQ ID NO: 189 - RNA, 30 nt, Artificial Sequence
  Synthetic Construct
ggugccuucgcaggcuguuuccuugcuuca

SEQ ID NO: 190 - RNA, 30 nt, Artificial Sequence
  Synthetic Construct
gugccuucgcaggcuguuuccuugcuucag

SEQ ID NO: 191 - RNA, 30 nt, Artificial Sequence
  Synthetic Construct
gcaggcuguuuccuugcuucaggaauggcc

SEQ ID NO: 192 - RNA, 30 nt, Artificial Sequence
  Synthetic Construct
cugaagcaaggaaacagccugcgaaggcac

SEQ ID NO: 193 - RNA, 30 nt, Artificial Sequence
  Synthetic Construct
ccugggcuggggaagaaggugucuucugga

SEQ ID NO: 194 - RNA, 30 nt, Artificial Sequence
  Synthetic Construct
ucccacagauauccagaacccugacccugc

SEQ ID NO: 195 - RNA, 30 nt, Artificial Sequence
  Synthetic Construct
uggaauaaugcuguuguugaaggcguuugc

SEQ ID NO: 196 - RNA, 30 nt, Artificial Sequence
  Synthetic Construct
uugaaggcguuugcacaugcaaagucagau

SEQ ID NO: 197 - RNA, 30 nt, Artificial Sequence
  Synthetic Construct
gcacaugcaaagucagauuuguugcuccag

SEQ ID NO: 198 - RNA, 30 nt, Artificial Sequence
  Synthetic Construct
cacaugcaaagucagauuuguugcuccagg

SEQ ID NO: 199 - RNA, 30 nt, Artificial Sequence
  Synthetic Construct
guugcuccaggccacagcacuguugcucuu

SEQ ID NO: 200 - RNA, 30 nt, Artificial Sequence
  Synthetic Construct
uugcuccaggccacagcacuguugcucuug

SEQ ID NO: 201 - RNA, 30 nt, Artificial Sequence
  Synthetic Construct
cuccaggccacagcacuguugcucuugaag

SEQ ID NO: 202 - RNA, 30 nt, Artificial Sequence
  Synthetic Construct
cucuugaaguccauagaccucaugucuagc

SEQ ID NO: 203 - RNA, 30 nt, Artificial Sequence
  Synthetic Construct
aaguccauagaccucaugucuagcacaguu

SEQ ID NO: 204 - RNA, 30 nt, Artificial Sequence
  Synthetic Construct
ugucugugauauacacaucagaauccuuac

SEQ ID NO: 205 - RNA, 30 nt, Artificial Sequence
  Synthetic Construct
gucugugauauacacaucagaauccuuacu

SEQ ID NO: 206 - RNA, 30 nt, Artificial Sequence
  Synthetic Construct
ucugugauauacacaucagaauccuuacuu

SEQ ID NO: 207 - RNA, 30 nt, Artificial Sequence
  Synthetic Construct
gugacacauuuguuugagaaucaaaaucgg

SEQ ID NO: 208 - RNA, 30 nt, Artificial Sequence
  Synthetic Construct
aaggcguuugcacaugcaaagucagauuug

SEQ ID NO: 209 - RNA, 30 nt, Artificial Sequence
  Synthetic Construct
ugacacauuuguuugagaaucaaaaucggu

SEQ ID NO: 210 - RNA, 30 nt, Artificial Sequence
  Synthetic Construct
guuugagaaucaaaaucggugaauaggcag

SEQ ID NO: 211 - RNA, 30 nt, Artificial Sequence
  Synthetic Construct
uuugagaaucaaaaucggugaauaggcaga

SEQ ID NO: 212 - RNA, 30 nt, Artificial Sequence
  Synthetic Construct
gagaaucaaaaucggugaauaggcagacag

SEQ ID NO: 213 - RNA, 30 nt, Artificial Sequence
  Synthetic Construct
agaaucaaaaucggugaauaggcagacaga

SEQ ID NO: 214 - RNA, 30 nt, Artificial Sequence
  Synthetic Construct
ucacuggauuuagagucucucagcugguac

SEQ ID NO: 215 - RNA, 30 nt, Artificial Sequence
  Synthetic Construct
agagucucucagcugguacacggcaggguc

SEQ ID NO: 216 - RNA, 30 nt, Artificial Sequence
  Synthetic Construct
gagucucucagcugguacacggcaggguca

SEQ ID NO: 217 - RNA, 30 nt, Artificial Sequence
  Synthetic Construct
uggauaucugugggacaagaggaucagggu

SEQ ID NO: 218 - RNA, 30 nt, Artificial Sequence
  Synthetic Construct
ggacaugaucucauuucccucuuugcccca

SEQ ID NO: 219 - RNA, 30 nt, Artificial Sequence
  Synthetic Construct
agcauccagagcaggcugacucucccaccc

SEQ ID NO: 220 - RNA, 30 nt, Artificial Sequence
  Synthetic Construct
uuccuuuuagaaaguuccugugaugucaag

SEQ ID NO: 221 - RNA, 30 nt, Artificial Sequence
  Synthetic Construct
cagcauccagagcaggcugacucucccacc

SEQ ID NO: 222 - RNA, 30 nt, Artificial Sequence
  Synthetic Construct
uaaaaggaaaaacagacauucuuucagcau

SEQ ID NO: 223 - RNA, 30 nt, Artificial Sequence
  Synthetic Construct
uccuuuuagaaaguuccugugaugucaagc

SEQ ID NO: 224 - RNA, 30 nt, Artificial Sequence
  Synthetic Construct
ccuuuuagaaaguuccugugaugucaagcu

SEQ ID NO: 225 - RNA, 30 nt, Artificial Sequence
  Synthetic Construct
cuuuuagaaaguuccugugaugucaagcug

SEQ ID NO: 226 - RNA, 30 nt, Artificial Sequence
  Synthetic Construct
uagaaaguuccugugaugucaagcuggucg

SEQ ID NO: 227 - RNA, 30 nt, Artificial Sequence
  Synthetic Construct
agaaaguuccugugaugucaagcuggucga

SEQ ID NO: 228 - RNA, 30 nt, Artificial Sequence
  Synthetic Construct
gaaaguuccugugaugucaagcuggucgag

SEQ ID NO: 229 - RNA, 30 nt, Artificial Sequence
  Synthetic Construct
cugugaugucaagcuggucgagaaaagcuu

SEQ ID NO: 230 - RNA, 30 nt, Artificial Sequence
  Synthetic Construct
gaaacagguaagacaggggucuagccuggg

SEQ ID NO: 231 - RNA, 30 nt, Artificial Sequence
  Synthetic Construct
aaacagguaagacaggggucuagccugggu

SEQ ID NO: 232 - RNA, 30 nt, Artificial Sequence
  Synthetic Construct
gcacaggauugcggaagugaugaacccgca

SEQ ID NO: 233 - RNA, 30 nt, Artificial Sequence
  Synthetic Construct
cgcaauccugugcaaacccaggcuagaccc

SEQ ID NO: 234 - RNA, 30 nt, Artificial Sequence
  Synthetic Construct
ccuguuucaaagcuuuucucgaccagcuug

SEQ ID NO: 235 - RNA, 30 nt, Artificial Sequence
  Synthetic Construct
caaagcuuuucucgaccagcuugacaucac

SEQ ID NO: 236 - RNA, 30 nt, Artificial Sequence
  Synthetic Construct
aaagcuuuucucgaccagcuugacaucaca

SEQ ID NO: 237 - RNA, 30 nt, Artificial Sequence
  Synthetic Construct
ucucgaccagcuugacaucacaggaacuuu

SEQ ID NO: 238 - RNA, 30 nt, Artificial Sequence
  Synthetic Construct
cucgaccagcuugacaucacaggaacuuuc

SEQ ID NO: 239 - RNA, 30 nt, Artificial Sequence
  Synthetic Construct
ucgaccagcuugacaucacaggaacuuucu

SEQ ID NO: 240 - RNA, 30 nt, Artificial Sequence
  Synthetic Construct
acaucacaggaacuuucuaaaaggaaaaac

SEQ ID NO: 241 - RNA, 30 nt, Artificial Sequence
  Synthetic Construct
cuaaaaggaaaaacagacauucuuucagca

SEQ ID NO: 242 - RNA, 30 nt, Artificial Sequence
  Synthetic Construct
uuucagcauccagagcaggcugacucuccc

SEQ ID NO: 243 - RNA, 30 nt, Artificial Sequence
  Synthetic Construct
cacaggauugcggaagugaugaacccgcaa

SEQ ID NO: 244 - RNA, 30 nt, Artificial Sequence
  Synthetic Construct
guuuuacagauacgaaccuaaacuuucaaa

SEQ ID NO: 245 - RNA, 30 nt, Artificial Sequence
  Synthetic Construct
guaucuguaaaaccaagaggccacagcggu

SEQ ID NO: 246 - RNA, 30 nt, Artificial Sequence
  Synthetic Construct
ugggcuugcaugucccaggccugcauggcc

SEQ ID NO: 247 - RNA, 30 nt, Artificial Sequence
  Synthetic Construct
uacagauacgaaccuaaacuuucaaaaccu

SEQ ID NO: 248 - RNA, 30 nt, Artificial Sequence
  Synthetic Construct
acagauacgaaccuaaacuuucaaaaccug

SEQ ID NO: 249 - RNA, 30 nt, Artificial Sequence
  Synthetic Construct
cagauacgaaccuaaacuuucaaaaccugu

SEQ ID NO: 250 - RNA, 30 nt, Artificial Sequence
  Synthetic Construct
aaaaccugucagugauuggguuccgaaucc

SEQ ID NO: 251 - RNA, 30 nt, Artificial Sequence
  Synthetic Construct
gguuccgaauccuccuccugaaaguggccg

SEQ ID NO: 252 - RNA, 30 nt, Artificial Sequence
  Synthetic Construct
cgaauccuccuccugaaaguggccggguuu

SEQ ID NO: 253 - RNA, 30 nt, Artificial Sequence
  Synthetic Construct
aaucugcucaugacgcugcggcuguggucc

SEQ ID NO: 254 - RNA, 30 nt, Artificial Sequence
  Synthetic Construct
aucugcucaugacgcugcggcuguggucca

SEQ ID NO: 255 - RNA, 30 nt, Artificial Sequence
  Synthetic Construct
aagcugggagugggguuuagggacgcgggu

SEQ ID NO: 256 - RNA, 30 nt, Artificial Sequence
  Synthetic Construct
caaaaccugucagugauuggguuccgaauc

SEQ ID NO: 257 - RNA, 30 nt, Artificial Sequence
  Synthetic Construct
aacccggccacuuucaggaggaggauucgg

SEQ ID NO: 258 - RNA, 30 nt, Artificial Sequence
  Synthetic Construct
caggaggaggauucggaacccaaucacuga

SEQ ID NO: 259 - RNA, 30 nt, Artificial Sequence
  Synthetic Construct
aggaggaggauucggaacccaaucacugac

SEQ ID NO: 260 - RNA, 30 nt, Artificial Sequence
  Synthetic Construct
ggaacccaaucacugacagguuuugaaagu

SEQ ID NO: 261 - RNA, 30 nt, Artificial Sequence
  Synthetic Construct
ugaaaguuuagguucguaucuguaaaacca

SEQ ID NO: 262 - RNA, 30 nt, Artificial Sequence
  Synthetic Construct
gaaaguuuagguucguaucuguaaaaccaa

SEQ ID NO: 263 - RNA, 30 nt, Artificial Sequence
  Synthetic Construct
aaaguuuagguucguaucuguaaaaccaag

SEQ ID NO: 264 - RNA, 30 nt, Artificial Sequence
  Synthetic Construct
agguucguaucuguaaaaccaagaggccac

SEQ ID NO: 265 - RNA, 30 nt, Artificial Sequence
  Synthetic Construct
aaggccccucaccucagcuggaccacagcc

SEQ ID NO: 266 - RNA, 30 nt, Artificial Sequence
  Synthetic Construct
gguucguaucuguaaaaccaagaggccaca

SEQ ID NO: 267 - RNA, 30 nt, Artificial Sequence
  Synthetic Construct
ucaagacguggggggaaauuaucucauuau

SEQ ID NO: 268 - RNA, 30 nt, Artificial Sequence
  Synthetic Construct
cuuuaugaacacuauuaaaaaaaaauaaac

SEQ ID NO: 269 - RNA, 30 nt, Artificial Sequence
  Synthetic Construct
agaagaagaauacuauguauuucuuuauga

SEQ ID NO: 270 - RNA, 30 nt, Artificial Sequence
  Synthetic Construct
cccccacgucuugagaagaagaauacuaug

SEQ ID NO: 271 - RNA, 30 nt, Artificial Sequence
  Synthetic Construct
ccccccacgucuugagaagaagaauacuau

SEQ ID NO: 272 - RNA, 30 nt, Artificial Sequence
  Synthetic Construct
augaaggcaucggcagcaggacauacaaca

SEQ ID NO: 273 - RNA, 30 nt, Artificial Sequence
  Synthetic Construct
uauguccugcugccgaugccuucauuaaaa

SEQ ID NO: 274 - RNA, 30 nt, Artificial Sequence
  Synthetic Construct
uaaugaaggcaucggcagcaggacauacaa

SEQ ID NO: 275 - RNA, 30 nt, Artificial Sequence
  Synthetic Construct
caaaucauuuuaaugaaggcaucggcagca

SEQ ID NO: 276 - RNA, 24 nt, Artificial Sequence
  Synthetic Construct
auuaaaaugauuuggaagagcaga

SEQ ID NO: 277 - RNA, 30 nt, Artificial Sequence
  Synthetic Construct
ucgaggcccugcuaugcuguguaucugggc

SEQ ID NO: 278 - RNA, 30 nt, Artificial Sequence
  Synthetic Construct
ucucauuaucgaggcccugcuaugcugugu

SEQ ID NO: 279 - RNA, 30 nt, Artificial Sequence
  Synthetic Construct
uuuaugaacacuauuaaaaaaaaauaaacc

SEQ ID NO: 280 - RNA, 30 nt, Artificial Sequence
  Synthetic Construct
aaugaaggcaucggcagcaggacauacaac

SEQ ID NO: 281 - RNA, 30 nt, Artificial Sequence
  Synthetic Construct
augaacacuauuaaaaaaaaauaaaccccu

SEQ ID NO: 282 - RNA, 30 nt, Artificial Sequence
  Synthetic Construct
ccgggggggcacagaggugguagcagcuuu

SEQ ID NO: 283 - RNA, 30 nt, Artificial Sequence
  Synthetic Construct
aaaaaaaauaaaccccucacaacauucugc

SEQ ID NO: 284 - RNA, 30 nt, Artificial Sequence
  Synthetic Construct
gagagggagaagaggggcaaugcagaggaa

SEQ ID NO: 285 - RNA, 30 nt, Artificial Sequence
  Synthetic Construct
ggagagggagaagaggggcaaugcagagga

SEQ ID NO: 286 - RNA, 30 nt, Artificial Sequence
  Synthetic Construct
ccucuguuuggagagggagaagaggggcaa

SEQ ID NO: 287 - RNA, 30 nt, Artificial Sequence
  Synthetic Construct
gggguaggagaguucccucuguuuggagag

SEQ ID NO: 288 - RNA, 30 nt, Artificial Sequence
  Synthetic Construct
accuccuuggggguaggagaguucccucug

SEQ ID NO: 289 - RNA, 30 nt, Artificial Sequence
  Synthetic Construct
caccuccuuggggguaggagaguucccucu

SEQ ID NO: 290 - RNA, 30 nt, Artificial Sequence
  Synthetic Construct
uucucaagacguggggggaaauuaucucau

SEQ ID NO: 291 - RNA, 30 nt, Artificial Sequence
  Synthetic Construct
guggcauugccgggggggcacagagguggu

SEQ ID NO: 292 - RNA, 30 nt, Artificial Sequence
  Synthetic Construct
ggguaggauccaguugguggcauugccggg

SEQ ID NO: 293 - RNA, 30 nt, Artificial Sequence
  Synthetic Construct
aucauaaauucggguaggauccaguuggug

SEQ ID NO: 294 - RNA, 30 nt, Artificial Sequence
  Synthetic Construct
agcaaucuuaaucauaaauucggguaggau

SEQ ID NO: 295 - RNA, 30 nt, Artificial Sequence
  Synthetic Construct
gcagcucuucagcaaucuuaaucauaaauu

SEQ ID NO: 296 - RNA, 30 nt, Artificial Sequence
  Synthetic Construct
ggcagcucuucagcaaucuuaaucauaaau

SEQ ID NO: 297 - RNA, 30 nt, Artificial Sequence
  Synthetic Construct
ccucugccgugaaugucaggcagugacaag

SEQ ID NO: 298 - RNA, 30 nt, Artificial Sequence
  Synthetic Construct
ugcaggaccuagagcccaagagaacccacu

SEQ ID NO: 299 - RNA, 30 nt, Artificial Sequence
  Synthetic Construct
ugaacacuauuaaaaaaaaauaaaccccuc

SEQ ID NO: 300 - RNA, 30 nt, Artificial Sequence
  Synthetic Construct
uucuucucaagacguggggggaaauuaucu

SEQ ID NO: 301 - RNA, 30 nt, Artificial Sequence
  Synthetic Construct
auuuuuuuuuaauaguguucauaaagaaau

SEQ ID NO: 302 - RNA, 30 nt, Artificial Sequence
  Synthetic Construct
auaguguucauaaagaaauacauaguauuc

SEQ ID NO: 303 - RNA, 30 nt, Artificial Sequence
  Synthetic Construct
cugaagagcugccaaacacugcugccaccc

SEQ ID NO: 304 - RNA, 30 nt, Artificial Sequence
  Synthetic Construct
agauugcugaagagcugccaaacacugcug

SEQ ID NO: 305 - RNA, 30 nt, Artificial Sequence
  Synthetic Construct
ugauuaagauugcugaagagcugccaaaca

SEQ ID NO: 306 - RNA, 30 nt, Artificial Sequence
  Synthetic Construct
augauuaagauugcugaagagcugccaaac

SEQ ID NO: 307 - RNA, 30 nt, Artificial Sequence
  Synthetic Construct
ucccucuccaaacagagggaacucuccuac

SEQ ID NO: 308 - RNA, 30 nt, Artificial Sequence
  Synthetic Construct
ccccucuucucccucuccaaacagagggaa

SEQ ID NO: 309 - RNA, 30 nt, Artificial Sequence
  Synthetic Construct
ccuuauugcugcuugucacugccugacauu

SEQ ID NO: 310 - RNA, 30 nt, Artificial Sequence
  Synthetic Construct
cucugcauugccccucuucucccucuccaa

SEQ ID NO: 311 - RNA, 30 nt, Artificial Sequence
  Synthetic Construct
cagaucugcaagauuguaagacagccugug

SEQ ID NO: 312 - RNA, 30 nt, Artificial Sequence
  Synthetic Construct
cucauuccagaucugcaagauuguaagaca

SEQ ID NO: 313 - RNA, 30 nt, Artificial Sequence
  Synthetic Construct
uuccucauuccagaucugcaagauuguaag

SEQ ID NO: 314 - RNA, 30 nt, Artificial Sequence
  Synthetic Construct
uguucuuccucauuccagaucugcaagauu

SEQ ID NO: 315 - RNA, 30 nt, Artificial Sequence
  Synthetic Construct
cucaguguccagccugacuucuguucuucc

SEQ ID NO: 316 - RNA, 30 nt, Artificial Sequence
  Synthetic Construct
caaucuugcagaucuggaaugaggaagaac

SEQ ID NO: 317 - RNA, 30 nt, Artificial Sequence
  Synthetic Construct
uaagacagccugugcucccucgcuccuucc

SEQ ID NO: 318 - RNA, 30 nt, Artificial Sequence
  Synthetic Construct
auaaagaaauacauaguauucuucuucuca

SEQ ID NO: 319 - RNA, 30 nt, Artificial Sequence
  Synthetic Construct
uugcugcuugucacugccugacauucacgg

SEQ ID NO: 320 - RNA, 30 nt, Artificial Sequence
  Synthetic Construct
ucacugccugacauucacggcagaggcaag

SEQ ID NO: 321 - RNA, 30 nt, Artificial Sequence
  Synthetic Construct
aauaguguucauaaagaaauacauaguauu

SEQ ID NO: 322 - RNA, 30 nt, Artificial Sequence
  Synthetic Construct
uaauaguguucauaaagaaauacauaguau

SEQ ID NO: 323 - RNA, 30 nt, Artificial Sequence
  Synthetic Construct
uuaauaguguucauaaagaaauacauagua

SEQ ID NO: 324 - RNA, 30 nt, Artificial Sequence
  Synthetic Construct
uuuaauaguguucauaaagaaauacauagu

SEQ ID NO: 325 - RNA, 30 nt, Artificial Sequence
  Synthetic Construct
uuuuaauaguguucauaaagaaauacauag

SEQ ID NO: 326 - RNA, 30 nt, Artificial Sequence
  Synthetic Construct
uuuuuaauaguguucauaaagaaauacaua

SEQ ID NO: 327 - RNA, 30 nt, Artificial Sequence
  Synthetic Construct
cugcuugucacugccugacauucacggcag

SEQ ID NO: 328 - RNA, 30 nt, Artificial Sequence
  Synthetic Construct
uuuuuuaauaguguucauaaagaaauacau

SEQ ID NO: 329 - RNA, 30 nt, Artificial Sequence
  Synthetic Construct
ugagggguuuauuuuuuuuuaauaguguuc

SEQ ID NO: 330 - RNA, 30 nt, Artificial Sequence
  Synthetic Construct
ggcucuagguccugcagaauguugugaggg

SEQ ID NO: 331 - RNA, 30 nt, Artificial Sequence
  Synthetic Construct
ucuugggcucuagguccugcagaauguugu

SEQ ID NO: 332 - RNA, 30 nt, Artificial Sequence
  Synthetic Construct
aguggguucucuugggcucuagguccugca

SEQ ID NO: 333 - RNA, 30 nt, Artificial Sequence
  Synthetic Construct
ccuccugcuccccagagacugccuccgcca

SEQ ID NO: 334 - RNA, 30 nt, Artificial Sequence
  Synthetic Construct
acggcagaggcaaggcugcugcagccuccc

SEQ ID NO: 335 - RNA, 30 nt, Artificial Sequence
  Synthetic Construct
uuuuuuuuuaauaguguucauaaagaaaua

SEQ ID NO: 336 - RNA, 30 nt, Artificial Sequence
  Synthetic Construct
cagaucuggaaugaggaagaacagaaguca

SEQ ID NO: 337 - DNA, 3162 bp, Artificial Sequence
  Synthetic Construct
atgagcagcgcgatcaaaagctacaagagcgttctgcgtccgaacgagcgtaagaaccaactgctgaaaagcaccattcagtgcctggaagacggtagcgcgttctttttcaagatgctgcaaggcctgtttggtggcatcaccccggagattgttcgtttcagcaccgaacaggagaaacagcaacaggatatcgcgctgtggtgcgcggttaactggttccgtccggtgagccaagacagcctgacccacaccattgcgagcgataacctggtggagaagtttgaggaatactatggtggcaccgcgagcgacgcgatcaaacagtacttcagcgcgagcattggcgaaagctactattggaacgactgccgtcaacagtactatgatctgtgccgtgagctgggtgttgaggtgagcgacctgacccatgatctggagatcctgtgccgtgaaaagtgcctggcggttgcgaccgagagcaaccagaacaacagcatcattagcgttctgtttggcaccggcgaaaaagaggaccgtagcgtgaaactgcgtatcaccaagaaaattctggaggcgatcagcaacctgaaagaaatcccgaagaacgttgcgccgattcaagagatcattctgaacgtggcgaaagcgaccaaggaaaccttccgtcaggtgtatgcgggtaacctgggtgcgccgagcaccctggagaaatttatcgcgaaggacggccaaaaagagttcgatctgaagaaactgcagaccgacctgaagaaagttattcgtggtaaaagcaaggagcgtgattggtgctgccaggaagagctgcgtagctacgtggagcaaaacaccatccagtatgacctgtgggcgtggggcgaaatgttcaacaaagcgcacaccgcgctgaaaatcaagagcacccgtaactacaactttgcgaagcaacgtctggaacagttcaaagagattcagagcctgaacaacctgctggttgtgaagaagctgaacgactttttcgatagcgaatttttcagcggcgaggaaacctacaccatctgcgttcaccatctgggtggcaaggacctgagcaaactgtataaggcgtgggaggatgatccggcggacccggaaaacgcgattgtggttctgtgcgacgatctgaaaaacaactttaagaaagagccgatccgtaacattctgcgttacatcttcaccattcgtcaagaatgcagcgcgcaggacatcctggcggcggcgaagtacaaccaacagctggatcgttataaaagccaaaaggcgaacccgagcgttctgggtaaccagggctttacctggaccaacgcggtgatcctgccggagaaggcgcagcgtaacgaccgtccgaacagcctggatctgcgtatttggctgtacctgaaactgcgtcacccggacggtcgttggaagaaacaccatatcccgttctacgatacccgtttcttccaagaaatttatgcggcgggcaacagcccggttgacacctgccagtttcgtaccccgcgtttcggttatcacctgccgaaactgaccgatcagaccgcgatccgtgttaacaagaaacatgtgaaagcggcgaagaccgaggcgcgtattcgtctggcgatccaacagggcaccctgccggtgagcaacctgaagatcaccgaaattagcgcgaccatcaacagcaaaggtcaagtgcgtattccggttaagtttgacgtgggtcgtcaaaaaggcaccctgcagatcggtgaccgtttctgcggctacgatcaaaaccagaccgcgagccacgcgtatagcctgtgggaagtggttaaagagggtcaataccataaagagctgggctgctttgttcgtttcatcagcagcggtgacatcgtgagcattaccgagaaccgtggcaaccaatttgatcagctgagctatgaaggtctggcgtacccgcaatatgcggactggcgtaagaaagcgagcaagttcgtgagcctgtggcagatcaccaagaaaaacaagaaaaaggaaatcgtgaccgttgaagcgaaagagaagtttgacgcgatctgcaagtaccagccgcgtctgtataaattcaacaaggagtacgcgtatctgctgcgtgatattgttcgtggcaaaagcctggtggaactgcaacagattcgtcaagagatctttcgtttcattgaacaggactgcggtgttacccgtctgggcagcctgagcctgagcaccctggaaaccgtgaaagcggttaagggtatcatttacagctattttagcaccgcgctgaacgcgagcaagaacaacccgatcagcgacgaacagcgtaaagagtttgatccggaactgttcgcgctgctggaaaagctggagctgattcgtacccgtaaaaagaaacaaaaagtggaacgtatcgcgaacagcctgattcagacctgcctggagaacaacatcaagttcattcgtggtgaaggcgacctgagcaccaccaacaacgcgaccaagaaaaaggcgaacagccgtagcatggattggttggcgcgtggtgtttttaacaaaatccgtcaactggcgccgatgcacaacattaccctgttcggttgcggcagcctgtacaccagccaccaggacccgctggtgcatcgtaacccggataaagcgatgaagtgccgttgggcggcgatcccggttaaggacattggcgattgggtgctgcgtaagctgagccaaaacctgcgtgcgaaaaacatcggcaccggcgagtactatcaccaaggtgttaaagagttcctgagccattatgaactgcaggacctggaggaagagctgctgaagtggcgtagcgatcgtaaaagcaacattccgtgctgggtgctgcagaaccgtctggcggagaagctgggcaacaaagaagcggtggtttacatcccggttcgtggtggccgtatttattttgcgacccacaaggtggcgaccggtgcggtgagcatcgttttcgaccaaaaacaagtgtgggtttgcaacgcggatcatgttgcggcggcgaacatcgcgctgaccgtgaagggtattggcgaacaaagcagcgacgaagagaacccggatggtagccgtatcaaactgcagctgaccagc

SEQ ID NO: 338 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 339 - DNA, 5114 bp, Homo sapiens
aacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttcattaaaatgatttggaagagcaga

SEQ ID NO: 340 - DNA, 373 bp, Homo sapiens
caaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatg

SEQ ID NO: 341 - DNA, 145 bp, Homo sapiens
gagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaa

SEQ ID NO: 342 - DNA, 208 bp, Homo sapiens
aggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctggg

SEQ ID NO: 343 - DNA, 602 bp, Homo sapiens
cctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttcattaaaatgatttggaagagcaga

SEQ ID NO: 344 - RNA, 43 nt, Artificial Sequence
  Synthetic Construct
agaaauccgucuuucauugacggaagagcaacagugcuguggc

SEQ ID NO: 345 - RNA, 43 nt, Artificial Sequence
  Synthetic Construct
agaaauccgucuuucauugacggaacaacagcauuauuccaga

SEQ ID NO: 346 - RNA, 43 nt, Artificial Sequence
  Synthetic Construct
agaaauccgucuuucauugacgggaaacagguaagacaggggu

SEQ ID NO: 347 - RNA, 43 nt, Artificial Sequence
  Synthetic Construct
agaaauccgucuuucauugacggcaggaggaggauucggaacc

SEQ ID NO: 348 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 349 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 350 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 351 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 352 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 353 - DNA, 3222 bp, Artificial Sequence
  Synthetic Construct
atggcttccatctctaggccatacggcaccaagctgcgaccggacgcacggaagaaggagatgctcgataagttctttaatacactgactaagggtcagcgcgtgttcgcagacctggccctgtgcatctatggctccctgaccctggagatggccaagtctctggagccagaaagtgattcagaactggtgtgcgctattgggtggtttcggctggtggacaagaccatctggtccaaggatggcatcaagcaggagaatctggtgaaacagtacgaagcctattccggaaaggaggcttctgaagtggtcaaaacatacctgaacagccccagctccgacaagtacgtgtggatcgattgcaggcagaaattcctgaggtttcagcgcgagctcggcactcgcaacctgtccgaggacttcgaatgtatgctctttgaacagtacattagactgaccaagggcgagatcgaagggtatgccgctatttcaaatatgttcggaaacggcgagaaggaagaccggagcaagaaaagaatgtacgctacacggatgaaagattggctggaggcaaacgaaaatatcacttgggagcagtatagagaggccctgaagaaccagctgaatgctaaaaacctggagcaggttgtggccaattacaaggggaacgctggcggggcagaccccttctttaagtatagcttctccaaagagggaatggtgagcaagaaagaacatgcacagcagctcgacaagttcaaaaccgtcctgaagaacaaagcccgggacctgaattttccaaacaaggagaagctgaagcagtacctggaggccgaaatcggcattccggtcgacgctaacgtgtactcccagatgttctctaacggggtgagtgaggtccagcctaagaccacacggaatatgtcttttagtaacgagaaactggatctgctcactgaactgaaggacctgaacaagggcgatgggttcgagtacgccagagaagtgctgaacgggttctttgactccgagctccacactaccgaggataagtttaatatcacctctaggtacctgggaggcgacaaatcaaaccgcctgagcaaactctataagatctggaagaaagagggtgtggactgcgaggaaggcattcagcagttctgtgaagccgtcaaagataagatgggccagatccccattcgaaatgtgctgaagtacctgtggcagttccgggagacagtcagtgccgaggattttgaagcagccgctaaggctaaccatctggaggaaaagatcagccgggtgaaagcccacccaatcgtgattagcaataggtactgggcttttgggacttccgcactggtgggaaacattatgcccgcagacaagaggcatcagggagagtatgccggtcagaatttcaaaatgtggctggaggctgaactgcactacgatggcaagaaagcaaagcaccatctgcctttttataacgcccgcttctttgaggaagtgtactgctatcacccctctgtcgccgagatcactcctttcaaaaccaagcagtttggctgtgaaatcgggaaggacattccagattacgtgagcgtcgctctgaaggacaatccgtataagaaagcaaccaaacgaatcctgcgtgcaatctacaatcccgtcgccaacacaactggcgttgataagaccacaaactgcagcttcatgatcaaacgcgagaatgacgaatataagctggtcatcaaccgaaaaatttccgtggatcggcctaagagaatcgaagtgggcaggacaattatggggtacgaccgcaatcagacagctagcgatacttattggattggccggctggtgccacctggaacccggggcgcataccgcatcggagagtggagcgtccagtatattaagtccgggcctgtcctgtctagtactcagggagttaacaattccactaccgaccagctggtgtacaacggcatgccatcaagctccgagcggttcaaggcctggaagaaagccagaatggcttttatccgaaaactcattcgtcagctgaatgacgagggactggaatctaagggtcaggattatatccccgagaacccttctagtttcgatgtgcggggcgaaaccctgtacgtctttaacagtaattatctgaaggccctggtgagcaaacacagaaaggccaagaaacctgttgaggggatcctggacgagattgaagcctggacatctaaagacaaggattcatgcagcctgatgcggctgagcagcctgagcgatgcttccatgcagggaatcgccagcctgaagagtctgattaacagctacttcaacaagaatggctgtaaaaccatcgaggacaaagaaaagtttaatcccgtgctgtatgccaagctggttgaggtggaacagcggagaacaaacaagcggtctgagaaagtgggaagaatcgcaggtagtctggagcagctggccctgctgaacggggttgaggtggtcatcggcgaagctgacctgggggaggtcgaaaaaggaaagagtaagaaacagaattcacggaacatggattggtgcgcaaagcaggtggcacagcggctggagtacaaactggccttccatggaatcggttactttggagtgaaccccatgtataccagccaccaggaccctttcgaacataggcgcgtggctgatcacatcgtcatgcgagcacgttttgaggaagtcaacgtggagaacattgccgaatggcacgtgcgaaatttctcaaactacctgcgtgcagacagcggcactgggctgtactataagcaggccaccatggacttcctgaaacattacggtctggaggaacacgctgagggcctggaaaataagaaaatcaagttctatgactttagaaagatcctggaggataaaaacctgacaagcgtgatcattccaaagaggggcgggcgcatctacatggccaccaacccagtgacatccgactctaccccgattacatacgccggcaagacttataataggtgtaacgctgatgaggtggcagccgctaatatcgttatttctgtgctggctccccgcagtaagaaaaacgaggaacaggacgatatccctctgattaccaagaaagccgagagtaagtcaccaccgaaagaccggaagagatcaaaaacaagccagctgcctcagaaa

SEQ ID NO: 354 - protein, 1074 aa, Artificial Sequence
  Synthetic Construct
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISVDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNEEQDDIPLITKKAESKSPPKDRKRSKTSQLPQK

SEQ ID NO: 355 - protein, 1074 aa, Artificial Sequence
  Synthetic Construct
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 356 - protein, 1074 aa, Artificial Sequence
  Synthetic Construct
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKHHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 357 - protein, 1093 aa, Artificial Sequence
  Synthetic Construct
MSNKEKNASETRKAYTTKMIPRSHDRMKLLGNFMDYLMDGTPIFFELWNQFGGGIDRDIISGTANKDKISDDLLLAVNWFKVMPINSKPQGVSPSNLANLFQQYSGSEPDIQAQEYFASNFDTEKHQWKDMRVEYERLLAELQLSRSDMHHDLKLMYKEKCIGLSLSTAHYITSVMFGTGAKNNRQTKHQFYSKVIQLLEESTQINSVEQLASIILKAGDCDSYRKLRIRCSRKGATPSILKIVQDYELGTNHDDEVNVPSLIANLKEKLGRFEYECEWKCMEKIKAFLASKVGPYYLGSYSAMLENALSPIKGMTTKNCKFVLKQIDAKNDIKYENEPFGKIVEGFFDSPYFESDTNVKWVLHPHHIGESNIKTLWEDLNAIHSKYEEDIASLSEDKKEKRIKVYQGDVCQTINTYCEEVGKEAKTPLVQLLRYLYSRKDDIAVDKIIDGITFLSKKHKVEKQKINPVIQKYPSFNFGNNSKLLGKIISPKDKLKHNLKCNRNQVDNYIWIEIKVLNTKTMRWEKHHYALSSTRFLEEVYYPATSENPPDALAARFRTKTNGYEGKPALSAEQIEQIRSAPVGLRKVKKRQMRLEAARQQNLLPRYTWGKDFNINICKRGNNFEVTLATKVKKKKEKNYKVVLGYDANIVRKNTYAAIEAHANGDGVIDYNDLPVKPIESGFVTVESQVRDKSYDQLSYNGVKLLYCKPHVESRRSFLEKYRNGTMKDNRGNNIQIDFMKDFEAIADDETSLYYFNMKYCKLLQSSIRNHSSQAKEYREEIFELLRDGKLSVLKLSSLSNLSFVMFKVAKSLIGTYFGHLLKKPKNSKSDVKAPPITDEDKQKADPEMFALRLALEEKRLNKVKSKKEVIANKIVAKALELRDKYGPVLIKGENISDTTKKGKKSSTNSFLMDWLARGVANKVKEMVMMHQGLEFVEVNPNFTSHQDPFVHKNPENTFRARYSRCTPSELTEKNRKEILSFLSDKPSKRPTNAYYNEGAMAFLATYGLKKNDVLGVSLEKFKQIMANILHQRSEDQLLFPSRGGMFYLATYKLDADATSVNWNGKQFWVCNADLVAAYNVGLVDIQKDFKKK

SEQ ID NO: 358 - protein, 1098 aa, Artificial Sequence
  Synthetic Construct
MSISNNNILPYNPKLLPDDRKHKMLVDTFNQLDLIRNNLHDMIIALYGALKYDNIKQFASKEKPHISADALCSINWFRLVKTNERKPAIESNQIISKFIQYSGHTPDKYALSHITGNHEPSHKWIDCREYAINYARIMHLSFSQFQDLATACLNCKILILNGTLTSSWAWGANSALFGGSDKENFSVKAKILNSFIENLKDEMNTTKFQVVEKVCQQIGSSDAADLFDLYRSTVKDGNRGPATGRNPKVMNLFSQDGEISSEQREDFIESFQKVMQEKNSKQIIPHLDKLKYHLVKQSGLYDIYSWAAAIKNANSTIVASNSSNLNTILNKTEKQQTFEELRKDEKIVACSKILLSVNDTLPEDLHYNPSTSNLGKNLDVFFDLLNENSVHTIENKEEKNKIVKECVNQYMEECKGLNKPPMPVLLTFISDYAHKHQAQDFLSAAKMNFIDLKIKSIKVVPTVHGSSPYTWISNLSKKNKDGKMIRTPNSSLIGWIIPPEEIHDQKFAGQNPIIWAVLRVYCNNKWEMHHFPFSDSRFFTEVYAYKPNLPYLPGGENRSKRFGYRHSTNLSNESRQILLDKSKYAKANKSVLRCMENMTHNVVFDPKTSLNIRIKTDKNNSPVLDDKGRITFVMQINHRILEKYNNTKIEIGDRILAYDQNQSENHTYAILQRTEEGSHAHQFNGWYVRVLETGKVTSIVQGLSGPIDQLNYDGMPVTSHKFNCWQADRSAFVSQFASLKISETETFDEAYQAINAQGAYTWNLFYLRILRKALRVCHMENINQFREEILAISKNRLSPMSLGSLSQNSLKMIRAFKSIINCYMSRMSFVDELQKKEGDLELHTIMRLTDNKLNDKRVEKINRASSFLTNKAHSMGCKMIVGESDLPVADSKTSKKQNVDRMDWCARALSHKVEYACKLMGLAYRGIPAYMSSHQDPLVHLVESKRSVLRPRFVVADKSDVKQHHLDNLRRMLNSKTKVGTAVYYREAVELMCEELGIHKTDMAKGKVSLSDFVDKFIGEKAIFPQRGGRFYMSTKRLTTGAKLICYSGSDVWLSDADEIAAINIGMFVVCDQTGAFKKKKKEKLDDEECDILPFRPM

SEQ ID NO: 359 - RNA, 36 nt, Artificial Sequence
  Synthetic Construct
ucucaacgauagucagacauguguccucagugacac

SEQ ID NO: 360 - RNA, 36 nt, Artificial Sequence
  Synthetic Construct
uuuuaacaacacucaggcauguguccacagugacac

SEQ ID NO: 361 - RNA, 36 nt, Artificial Sequence
  Synthetic Construct
uugaacggauacucagacauguguuuccagugacac

SEQ ID NO: 362 - RNA, 36 nt, Artificial Sequence
  Synthetic Construct
ugcccucaauagucagauguguguccacagugacac

SEQ ID NO: 363 - RNA, 36 nt, Artificial Sequence
  Synthetic Construct
ucucaaugauacuuagauacguguccucagugacac

SEQ ID NO: 364 - RNA, 36 nt, Artificial Sequence
  Synthetic Construct
ucucaaugauacucagacauguguccccagugacac

SEQ ID NO: 365 - RNA, 36 nt, Artificial Sequence
  Synthetic Construct
ucucaaugauacuaagacauguguccucagugacac

SEQ ID NO: 366 - RNA, 36 nt, Artificial Sequence
  Synthetic Construct
ucucaacuauacucagacauguguccucagugacac

SEQ ID NO: 367 - RNA, 36 nt, Artificial Sequence
  Synthetic Construct
ucucaacgauacucagacauguguccucagugacac

SEQ ID NO: 368 - RNA, 36 nt, Artificial Sequence
  Synthetic Construct
ucucaacgauacuaagauauguguccucagcgacac

SEQ ID NO: 369 - RNA, 36 nt, Artificial Sequence
  Synthetic Construct
ucucaacgauacuaagauauguguccccagugacac

SEQ ID NO: 370 - RNA, 36 nt, Artificial Sequence
  Synthetic Construct
ucucaacgauacuaagauauguguccacagugacac

SEQ ID NO: 371 - RNA, 36 nt, Artificial Sequence
  Synthetic Construct
ucucaacaauacucagacauguguccccagugacac

SEQ ID NO: 372 - RNA, 36 nt, Artificial Sequence
  Synthetic Construct
ucucaacaauacuaaggcauguguccccagugaccc

SEQ ID NO: 373 - RNA, 36 nt, Artificial Sequence
  Synthetic Construct
ucucaaagauacucagacacguguccccagugacac

SEQ ID NO: 374 - RNA, 36 nt, Artificial Sequence
  Synthetic Construct
ucucaaaaauacucagacauguguccucagugacac

SEQ ID NO: 375 - RNA, 36 nt, Artificial Sequence
  Synthetic Construct
gcgaaacaacagucagacauguguccccagugacac

SEQ ID NO: 376 - RNA, 36 nt, Artificial Sequence
  Synthetic Construct
ccucaacgauauuaagacauguguccgcagugacac

SEQ ID NO: 377 - RNA, 22 nt, Artificial Sequence
  Synthetic Construct
agacauguguccucagugacac

SEQ ID NO: 378 - RNA, 36 nt, Artificial Sequence
  Synthetic Construct
guuggaaugacuaauuuuugugcccaccguuggcac

SEQ ID NO: 379 - RNA, 24 nt, Artificial Sequence
  Synthetic Construct
aauuuuugugcccaucguuggcac

SEQ ID NO: 380 - RNA, 23 nt, Artificial Sequence
  Synthetic Construct
auuuuugugcccaucguuggcac

SEQ ID NO: 381 - RNA, 36 nt, Artificial Sequence
  Synthetic Construct
cuagcaaugaccuaauaguguguccuuaguugacau

SEQ ID NO: 382 - RNA, 36 nt, Artificial Sequence
  Synthetic Construct
ccuacaauaccuaagaaauccguccuaaguugacgg

SEQ ID NO: 383 - RNA, 22 nt, Artificial Sequence
  Synthetic Construct
auaguguguccuuaguugacau

SEQ ID NO: 384 - RNA, 100 nt, Artificial Sequence
  Synthetic Construct
  misc_feature (1)..(4): The linkages between nucleotides at positions 1, 2, 3, and 4 are phosphorothioate linkages
  modified_base (1)..(2): 2′-O-methyladenosine
  modified_base (3)..(3): gm
  misc_feature (97)..(100): The linkages between nucleotides at positions 97, 98, 99, and 100 are phosphorothioate linkages
  modified_base (97)..(99): um
aaguuccugugaugucaagcguuuuagagcuagaaauagcaaguuaaaauaaggcuaguccguuaucaacuugaaaaaguggcaccgagucggugcuuuu

SEQ ID NO: 385 - RNA, 43 nt, Artificial Sequence
  Synthetic Construct
agaaauccgucuuucauugacgguggauaucugugggacaaga

SEQ ID NO: 386 - RNA, 43 nt, Artificial Sequence
  Synthetic Construct
agaaauccgucuuucauugacgggugacacauuuguuugagaa

SEQ ID NO: 387 - RNA, 43 nt, Artificial Sequence
  Synthetic Construct
agaaauccgucuuucauugacgggcaugugcaaacgccuucaa

SEQ ID NO: 388 - RNA, 43 nt, Artificial Sequence
  Synthetic Construct
agaaauccgucuuucauugacgguggaauaaugcuguuguuga

SEQ ID NO: 389 - RNA, 43 nt, Artificial Sequence
  Synthetic Construct
agaaauccgucuuucauugacgguuccccagcccagguaaggg

SEQ ID NO: 390 - RNA, 43 nt, Artificial Sequence
  Synthetic Construct
agaaauccgucuuucauugacggcccagcccagguaagggcag

SEQ ID NO: 391 - RNA, 43 nt, Artificial Sequence
  Synthetic Construct
agaaauccgucuuucauugacggcuaaaaggaaaaacagacau

SEQ ID NO: 392 - RNA, 43 nt, Artificial Sequence
  Synthetic Construct
agaaauccgucuuucauugacggcugugaugucaagcuggucg

SEQ ID NO: 393 - RNA, 43 nt, Artificial Sequence
  Synthetic Construct
agaaauccgucuuucauugacggucucgaccagcuugacauca

SEQ ID NO: 394 - RNA, 43 nt, Artificial Sequence
  Synthetic Construct
agaaauccgucuuucauugacggguaucuguaaaaccaagagg

SEQ ID NO: 395 - RNA, 43 nt, Artificial Sequence
  Synthetic Construct
agaaauccgucuuucauugacggcaaaaccugucagugauugg

SEQ ID NO: 396 - RNA, 43 nt, Artificial Sequence
  Synthetic Construct
agaaauccgucuuucauugacggcgaauccuccuccugaaagu

SEQ ID NO: 397 - RNA, 43 nt, Artificial Sequence
  Synthetic Construct
agaaauccgucuuucauugacggaaggccccucaccucagcug

SEQ ID NO: 398 - DNA, 20 bp, Artificial Sequence
  Synthetic Construct
tggatatctgtgggacaaga

SEQ ID NO: 399 - DNA, 20 bp, Artificial Sequence
  Synthetic Construct
gtgacacatttgtttgagaa

SEQ ID NO: 400 - DNA, 20 bp, Artificial Sequence
  Synthetic Construct
aagagcaacagtgctgtggc

SEQ ID NO: 401 - DNA, 20 bp, Artificial Sequence
  Synthetic Construct
gcatgtgcaaacgccttcaa

SEQ ID NO: 402 - DNA, 20 bp, Artificial Sequence
  Synthetic Construct
aacaacagcattattccaga

SEQ ID NO: 403 - DNA, 20 bp, Artificial Sequence
  Synthetic Construct
tggaataatgctgttgttga

SEQ ID NO: 404 - DNA, 20 bp, Artificial Sequence
  Synthetic Construct
ttccccagcccaggtaaggg

SEQ ID NO: 405 - DNA, 20 bp, Artificial Sequence
  Synthetic Construct
cccagcccaggtaagggcag

SEQ ID NO: 406 - DNA, 20 bp, Artificial Sequence
  Synthetic Construct
ctaaaaggaaaaacagacat

SEQ ID NO: 407 - DNA, 20 bp, Artificial Sequence
  Synthetic Construct
ctgtgatgtcaagctggtcg

SEQ ID NO: 408 - DNA, 20 bp, Artificial Sequence
  Synthetic Construct
tctcgaccagcttgacatca

SEQ ID NO: 409 - DNA, 20 bp, Artificial Sequence
  Synthetic Construct
gaaacaggtaagacaggggt

SEQ ID NO: 410 - DNA, 20 bp, Artificial Sequence
  Synthetic Construct
gtatctgtaaaaccaagagg

SEQ ID NO: 411 - DNA, 20 bp, Artificial Sequence
  Synthetic Construct
caaaacctgtcagtgattgg

SEQ ID NO: 412 - DNA, 20 bp, Artificial Sequence
  Synthetic Construct
cgaatcctcctcctgaaagt

SEQ ID NO: 413 - DNA, 20 bp, Artificial Sequence
  Synthetic Construct
caggaggaggattcggaacc

SEQ ID NO: 414 - DNA, 20 bp, Artificial Sequence
  Synthetic Construct
aaggcccctcacctcagctg

(19) United States
(12) Patent Application Publication
(10) Pub. No.: US 2024/0026351 A1
(43) Pub. Date: Jan. 25, 2024

(54) COMPOSITIONS COMPRISING AN RNA GUIDE TARGETING TRAC AND USES THEREOF

(71) Applicant: ARBOR BIOTECHNOLOGIES, INC., CAMBRIDGE, MA (US)

(72) Inventor: Quinton Norman Wessells, (US)

(73) Assignee: Arbor Biotechnologies, Inc., Cambridge, MA (US)

(21) Appl. No.: 18/034,539

(22) PCT Filed: Oct. 29, 2021
(86) PCT No.: PCT/US2021/057404

§ 371 Date: Apr. 28, 2023

(51) Int. Cl. C12N 15/11; C12N 9/22; C12N 15/90; C07K 14/725

(52) U.S. Cl. CPC C12N 15/11; C12N 9/22; C12N 15/907; C12N 15/111; C07K 14/7051; C12N 2310/20; C12N 2800/80

(57) ABSTRACT

The present invention relates to compositions comprising RNA guides targeting TRAC, processes for characterizing the compositions, cells comprising the compositions, and methods of using the compositions.

### SEQUENCE LISTING

The instant application contains a Sequence Listing which has been submitted electronically in ASCII format and is hereby incorporated by reference in its entirety. Said ASCII copy, created on Oct. 29, 2021, is named 51451-015WO3_Sequence_Listing_10_29_21_ST25, and is 196,584 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art. Although this invention disclosed herein is not limited to specific advantages or functionalities, the invention provides a composition comprising an RNA guide, wherein the RNA guide comprises (i) a spacer sequence that is substantially complementary to a target sequence within a TRAC gene and (ii) a direct repeat sequence: wherein the target sequence is adjacent to a protospacer adjacent motif (PAM) comprising the sequence 5′-NTTN-3′.

In one aspect of the composition, the target sequence is within exon 1, exon 2, exon 3, or exon 4 of the TRAC gene.

In another aspect of the composition, the TRAC gene sequence comprises the sequence of SEQ ID NO: 339, the reverse complement of SEQ ID NO: 339, a variant of the sequence of SEQ ID NO: 339, or the reverse complement of a variant of SEQ ID NO: 339.

In another aspect of the composition, the spacer sequence comprises: a. nucleotide 1 through nucleotide 16 of a sequence that is at least 90% identical to a sequence of any one of SEQ ID NOs: 174-336; b. nucleotide 1 through nucleotide 17 of a sequence that is at least 90% identical to a sequence of any one of SEQ ID NOs: 174-336; c. nucleotide 1 through nucleotide 18 of a sequence that is at least 90% identical to a sequence of any one of SEQ ID NOs: 174-336; d. nucleotide 1 through nucleotide 19 of a sequence that is at least 90% identical to a sequence of any one of SEQ ID NOs: 174-336; e. nucleotide 1 through nucleotide 20 of a sequence that is at least 90% identical to a sequence of any one of SEQ ID NOs: 174-336; f. nucleotide 1 through nucleotide 21 of a sequence that is at least 90% identical to a sequence of any one of SEQ ID NOs: 174.336; g. nucleotide 1 through nucleotide 22 of a sequence that is at least 90% identical to a sequence of any one of SEQ ID NOs: 174-336, h. nucleotide 1 through nucleotide 23 of a sequence that is at least 90% identical to a sequence of any one of SEQ ID NOs: 174-336; i. nucleotide 1 through nucleotide 24 of a sequence that is at least 90% identical to a sequence of any one of SEQ ID NOs. 174-336; j. nucleotide 1 through nucleotide 25 of a sequence that is at least 90% identical to a sequence of any one of SEQ ID NOs: 174-275 and 277-336; k. nucleotide 1 through nucleotide 26 of a sequence that is at least 90% identical to a sequence of any one of SEQ ID NOs: 174-275 and 277-336; l. nucleotide 1 through nucleotide 27 of a sequence that is at least 90% identical to a sequence of any one of SEQ ID NOs: 174-275 and 277-336; m. nucleotide 1 through nucleotide 28 of a sequence that is at least 90% identical to a sequence of any one of SEQ ID NOs: 174-275 and 277-336; n. nucleotide 1 through nucleotide 29 of a sequence that is at least 90% identical to a sequence of any one of SEQ ID NOs: 174-275 and 277-336; or o. nucleotide 1 through nucleotide 30 of a sequence that is at least 90% identical to a sequence of any one of SEQ ID NOs: 174-275 and 277-336.

In another aspect of the composition, the spacer sequence comprises: a. nucleotide 1 through nucleotide 16 of any one of SEQ ID NOs: 174-336; b. nucleotide 1 through nucleotide 17 of any one of SEQ ID NOs: 174-336; c. nucleotide 1 through nucleotide 18 of any one of SEQ ID NOs: 174-336; d. nucleotide 1 through nucleotide 19 of any one of SEQ ID NOs: 174-336; e. nucleotide 1 through nucleotide 20 of any one of SEQ 10 NOs: 174-336; f. nucleotide 1 through nucleotide 21 of any one of SEQ 10 NOs: 174-336; g. nucleotide 1 through nucleotide 22 of any one of SEQ ID NOs: 174-336: h. nucleotide 1 through nucleotide 23 of any one of SEQ ID NOs: 174-336; i. nucleotide 1 through nucleotide 24 of any one of SEQ ID NOs: 174-336; j. nucleotide 1 through nucleotide 25 of any one of SEQ ID NOs: 174-275 and 277-336; k. nucleotide 1 through nucleotide 26 of any one of SEQ ID NOs: 174-275 and 277-336; l. nucleotide 1 through nucleotide 27 of any one of SEQ ID NOs: 174-275 and 277-336; m. nucleotide 1 through nucleotide 28 of any one of SEQ ID NOs: 174-275 and 277-336; n. nucleotide 1 through nucleotide 29 of any one of SEQ ID NOs: 174-275 and 277-336; or o. nucleotide 1 through nucleotide 30 of any one of SEQ ID NOs: 174-275 and 277-336.

In another aspect of the composition, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8; d. nucleotide 4 through nucleotide 36 of a sequence that is at least 90% identical to a sequence of any one of SEQ ID NOs: 1-8; e. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8: g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8: j. nucleotide 10 through nucleotide 36 of a sequence that is at least 90% identical to a sequence of any one of SEQ ID NOs: 1-8; k. nucleotide 11 through nucleotide 36 of a sequence that is at least 90% identical to a sequence of any one of SEQ ID NOs: 1-8; l. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 through nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 11 through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In another aspect of the composition, the direct repeat comprises: a. nucleotide 1 through nucleotide 36 of any one of SEQ ID NOs: 1-8; b. nucleotide 2 through nucleotide 36 of any one of SEQ ID NOs: 1-8: c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8: h. nucleotide 8 through nucleotide 36 of any one of SEQ ID NOs: 1-8; i. nucleotide 9 through nucleotide 36 of any one of SEQ ID NOs: 1-8; j. nucleotide 10 through nucleotide 36 of any one of SEQ ID NOs: 1-8; k. nucleotide 11 through nucleotide 36 of any one of SEQ ID NOs: 1-8; l. nucleotide 12 through nucleotide 36 of any one. of SEQ ID NOs: 1-8; n. nucleotide 13 through nucleotide 36 of any one of SEQ ID NOs: 1-8; n. nucleotide 14 through nucleotide 36 of any one of SEQ ID NOs: 1-8; o. nucleotide 1 through nucleotide 34 of SEQ ID NO: 9; p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9: t. nucleotide 6 through nucleotide 34 of SEQ ID NO: 9; u. nucleotide 7 through nucleotide 34 of SEQ ID NO: 9; v. nucleotide 8 through nucleotide 34 of SEQ ID NO: 9; w. nucleotide 9 through nucleotide 34 of SEQ ID NO: 9: x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9; or aa. SEQ ID NO: 10 or a portion thereof.

In another aspect of the composition, the direct repeat comprises: a. nucleotide 1 through nucleotide 36 of a sequence that is at least 90% identical to a sequence of any one of SEQ ID NOs: 359-376; b. nucleotide 2 through nucleotide 36 of a sequence that is at least 90% identical to a sequence of any one of SEQ ID NOs: 359-376; c. nucleotide 3 through nucleotide 36 of a sequence that is at least 90% identical to a sequence of any one of SEQ 10 NOs: 359-376; d. nucleotide 4 through nucleotide 36 of a sequence that is at least 90% identical to a sequence of any one of SEQ ID NOs: 359-376; c. nucleotide 5 through nucleotide 36 of a sequence that is at least 90% identical to a sequence of any one of SEQ ID NOs: 359-376; f. nucleotide 6 through nucleotide 36 of a sequence that is at least 90% identical to a sequence of any one of SEQ ID NOs: 359-376: g. nucleotide 7 through nucleotide 36 of a sequence that is at least 90% identical to a sequence of any one of SEQ ID NOs: 359-376; h. nucleotide 8 through nucleotide 36 of a sequence that is at least 90% identical to a sequence of any one of SEQ ID NOs: 359-376; i. nucleotide 9 through nucleotide 36 of a sequence that is at least 90% identical to a sequence of any one of SEQ ID NOs: 359-376; j. nucleotide 10 through nucleotide 36 of a sequence that is at least 90% identical to a sequence of any one of SEQ ID NOs: 359-376; k. nucleotide 11 through nucleotide 36 of a sequence that is at least 90% identical to a sequence of any one of SEQ ID NOs: 359-376; l. nucleotide 12 through nucleotide 36 of a sequence that is at least 90% identical to a sequence of any one of SEQ ID NOs: 359-376: m. nucleotide 13 through nucleotide 36 of a sequence that is at least 90% identical to a sequence of any one of SEQ ID NOs: 359-376: n. nucleotide 14 through nucleotide 36 of a sequence that is at least 90% identical to a sequence of any one of SEQ ID NOs: 359-376; or o. a sequence that is at least 90% identical to a sequence of SEQ ID NO: 377 or a portion thereof.

In another aspect of the composition, the direct repeat comprises: a. nucleotide 1 through nucleotide 36 of any one of SEQ ID NOs: 359-376; b. nucleotide 2 through nucleotide 36 of any one of SEQ ID NOs: 359-376; c. nucleotide 3 through nucleotide 36 of any one of SEQ ID NOs: 359-376; d. nucleotide 4 through nucleotide 36 of any one of SEQ ID NOs: 359-376; e. nucleotide 5 through nucleotide 36 of any one of SEQ ID NOs: 359-376; f. nucleotide 6 through nucleotide 36 of any one of SEQ ID NOs: 359-376; g. nucleotide 7 through nucleotide 36 of any one of SEQ ID NOs: 359-376; h. nucleotide 8 through nucleotide 36 of any one of SEQ ID NOs: 359-376; i. nucleotide 9 through nucleotide 36 of any one of SEQ ID NOs: 359-376; j. nucleotide 10 through nucleotide 36 of any one of SEQ ID NOs: 359-376; k. nucleotide 11 through nucleotide 36 of any one of SEQ ID NOs: 359-376; I. nucleotide 12 through nucleotide 36 of any one of SEQ ID NOs: 359-376; m. nucleotide 13 through nucleotide 36 of any one of SEQ ID NOs: 359-376; n. nucleotide 14 through nucleotide 36 of any one of SEQ ID NOs: 359-376; or o. SEQ ID NO: 377 or a portion thereof.

In another aspect of the composition, the direct repeat comprises: a. nucleotide 1 through nucleotide 36 of a sequence that is at least 90% identical to SEQ ID NO: 378; b. nucleotide 2 through nucleotide 36 of a sequence that is at least 90% identical to SEQ ID NO: 378; c. nucleotide 3 through nucleotide 36 of a sequence that is at least 90% identical to SEQ ID NO: 378; d. nucleotide 4 through nucleotide 36 of a sequence that is at least 90% identical to SEQ ID NO: 378; e. nucleotide 5 through nucleotide 36 of a sequence that is at least 90% identical to SEQ ID NO: 378; f. nucleotide 6 through nucleotide 36 of a sequence that is at least 90% identical to SEQ ID NO: 378; g. nucleotide 7 through nucleotide 36 of a sequence that is at least 90% identical to SEQ ID NO: 378; h. nucleotide 8 through nucleotide 36 of a sequence that is at least 90% identical to SEQ ID NO: 378; i. nucleotide 9 through nucleotide 36 of a sequence that is at least 90% identical to SEQ ID NO: 378; j. nucleotide 10 through nucleotide 36 of a sequence that is at least 90% identical to SEQ ID NO: 378; k. nucleotide 11 through nucleotide 36 of a sequence that is at least 90% identical to SEQ ID NO: 378; l. nucleotide 12 through nucleotide 36 of a sequence that is at least 90% identical to SEQ ID NO: 378; m. nucleotide 13 through nucleotide 36 of a sequence that is at least 90% identical to SEQ ID NO: 378; n. nucleotide 14 through nucleotide 36 of a sequence that is at least 90% identical to SEQ ID NO: 378; or o. a sequence that is at least 90% identical to a sequence of SEQ ID NO: 379 or SEQ ID NO: 380 or a portion thereof.

In another aspect of the composition, the direct repeat comprises: a. nucleotide 1 through nucleotide 36 of SEQ ID NO: 378; b. nucleotide 2 through nucleotide 36 of SEQ ID NO: 378; c. nucleotide 3 through nucleotide 36 of SEQ ID NO: 378; d. nucleotide 4 through nucleotide 36 of SEQ ID NO: 378; c. nucleotide 5 through nucleotide 36 of SEQ ID NO: 378; f. nucleotide 6 through nucleotide 36 of SEQ ID NO: 378; g. nucleotide 7 through nucleotide 36 of SEQ IL) NO: 378; h. nucleotide 8 through nucleotide 36 of SEQ ID NO: 378; i. nucleotide 9 through nucleotide 36 of SEQ ID NO: 378; j. nucleotide 10 through nucleotide 36 of SEQ ID NO: 378; k. nucleotide 11 through nucleotide 36 of SEQ ID NO: 378; l. nucleotide 12 through nucleotide 36 of SEQ ID NO: 378; m. nucleotide 13 through nucleotide 36 of SEQ ID NO: 378; n. nucleotide 14 through nucleotide 36 of SEQ ID NO: 378; or o. SEQ ID NO: 379 or SEQ ID NO: 380 or a portion thereof.

In another aspect of the composition, the direct repeat comprises: a. nucleotide 1 through nucleotide 36 of a sequence that is at least 90% identical to a sequence of SEQ ID NO: 381 or SEQ ID NO: 382; b. nucleotide 2 through nucleotide 36 of a sequence that is at least 90% identical to a sequence of SEQ ID NO: 381 or SEQ ID NO: 382; c. nucleotide 3 through nucleotide 36 of a sequence that is at least 90% identical to a sequence of SEQ ID NO: 381 or SEQ ID NO: 382: d. nucleotide 4 through nucleotide 36 of a sequence that is at least 90% identical to a sequence of SEQ ID NO: 381 or SEQ ID NO: 382; e. nucleotide 5 through nucleotide 36 of a sequence that is at least 90% identical to a sequence of SEQ ID NO: 381 or SEQ ID NO: 382; f. nucleotide 6 through nucleotide 36 of a sequence that is at least 90% identical to a sequence of SEQ ID NO: 381 or SEQ ID NO: 382: g. nucleotide 7 through nucleotide 36 of a sequence that is at least 90% identical to a sequence of SEQ ID NO: 381 or SEQ ID NO: 382; h. nucleotide 8 through nucleotide 36 of a sequence that is at least 90% identical to a sequence of SEQ ID NO: 381 or SEQ ID NO: 382; i. nucleotide 9 through nucleotide 36 of a sequence that is at least 90% identical to a sequence of SEQ ID NO: 381 or SEQ ID NO: 382; j. nucleotide 10 through nucleotide 36 of a sequence that is at least 90% identical to a sequence of SEQ ID NO: 381 or SEQ ID NO: 382; k. nucleotide 11 through nucleotide 36 of a sequence that is at least 90% identical to a sequence of SEQ ID NO: 381 or SEQ ID NO: 382; l. nucleotide 12 through nucleotide 36 of a sequence that is at least 90% identical to a sequence of SEQ ID NO: 381 or SEQ ID NO: 382; m. nucleotide 13 through nucleotide 36 of a sequence that is at least 90% identical to a sequence of SEQ ID NO: 381 or SEQ ID NO: 382; n. nucleotide 14 through nucleotide 36 of a sequence that is at least 90% identical to a sequence of SEQ ID NO: 381 or SEQ ID NO: 382; o. nucleotide 15 through nucleotide 36 of a sequence that is at least 90% identical to a sequence of SEQ ID NO: 381 or SEQ ID NO: 382; or p. a sequence that is at least 90% identical to a sequence of SEQ ID NO: 383 or a portion thereof.

In another aspect of the composition, the direct repeat comprises: a. nucleotide 1 through nucleotide 36 of SEQ ID NO: 381 or SEQ ID NO: 382: h. nucleotide 2 through nucleotide 36 of SEQ ID NO: 381 or SEQ ID NO: 382; c. nucleotide 3 through nucleotide 36 of SEQ ID NO: 381 or SEQ ID NO: 382; d. nucleotide 4 through nucleotide 36 of SEQ ID NO: 381 or SEQ ID NO: 382; e. nucleotide 5 through nucleotide 36 of SEQ ID NO: 381 or SEQ ID NO: 382; f. nucleotide 6 through nucleotide 36 of SEQ ID NO: 381 or SEQ ID NO: 382; g. nucleotide 7 through nucleotide 36 of SEQ ID NO: 381 or SEQ ID NO: 382; h. nucleotide 8 through nucleotide 36 of SEQ ID NO: 381 or SEQ ID NO: 382; i. nucleotide 9 through nucleotide 36 of SEQ ID NO: 381 or SEQ ID NO: 382; j. nucleotide 10 through nucleotide 36 of SEQ ID NO: 381 or SEQ ID NO: 382; k. nucleotide 11 through nucleotide 36 of SEQ ID NO: 381 or SEQ ID NO: 382; 1. nucleotide 12 through nucleotide 36 of SEQ ID NO: 381 or SEQ ID NO: 382; m. nucleotide 13 through nucleotide 36 of SEQ ID NO: 381 or SEQ ID NO: 382; n. nucleotide 14 through nucleotide 36 of SEQ ID NO: 381 or SEQ ID NO: 382; o. nucleotide 15 through nucleotide 36 of SEQ ID NO: 381 or SEQ ID NO: 382; or p. SEQ ID NO: 383 or a portion thereof.

In another aspect of the composition, the spacer sequence is substantially complementary to the complement of a sequence of any one of SEQ ID NOs: 11-173.

In another aspect of the composition, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In another aspect of the composition, the target sequence is immediately adjacent to the PAM sequence.

In another aspect of the composition, the composition further comprises a Cas12i polypeptide.

In another aspect of the composition, the Cas12i polypeptide iv: a. a Cas12i2 polypeptide comprising a sequence that is at least 90% identical to the sequence of SEQ ID NO: 338, SEQ ID NO: 348, SEQ ID NO: 349, SEQ ID NO: 350, SEQ ID NO: 351, or SEQ ID NO: 352; b. a Cas12i4 polypeptide comprising a sequence that is at least 90% identical to the sequence of SEQ ID NO: 354, SEQ ID NO: 355. or SEQ ID NO: 356; c. a Cas12i1 polypeptide comprising a sequence that is at least 90% identical to the sequence of SEQ ID NO: 357; or d. a Cas12i3 polypeptide comprising a sequence that is at least 90% identical to the sequence of SEQ ID NO: 358.

In another aspect of the composition, the Cas12i polypeptide is: a. a Cas12i2 polypeptide comprising a sequence of SEQ ID NO: 338, SEQ ID NO: 348, SEQ ID NO: 349, SEQ ID NO: 350, SEQ ID NO: 351, or SEQ ID NO: 352; b. a Cas12i4 polypeptide comprising a sequence of SEQ ID NO: 354, SEQ ID NO: 355, or SEQ ID NO: 356; c. a Cas12i1 polypeptide comprising a sequence of SEQ ID NO: 357; or d. a Cas12i3 polypeptide comprising a sequence of SEQ ID NO: 358.

In another aspect of the composition, the RNA guide and the Cas12i polypeptide form a ribonucleoprotein complex.

In another aspect of the composition, the ribonucleoprotein complex binds a target nucleic acid.

In another aspect of the composition, the composition is present within a cell.

In another aspect of the composition, the RNA guide and the Cas12i polypeptide are encoded in a vector, e.g., expression vector. In another aspect of the composition, the RNA guide and the Cas12i polypeptide are encoded in a single vector or the RNA guide is encoded in a first vector and the Cas12i polypeptide is encoded in a second vector.

The invention further provides a vector system comprising one or more vectors encoding an RNA guide disclosed herein and a Cas12i polypeptide. In an embodiment, the vector system comprises a first vector encoding an RNA guide disclosed herein and a second vector encoding a Cas12i polypeptide. The vectors may be expression vectors.

The invention further provides a composition comprising an RNA guide and a Cas12i polypeptide, wherein the RNA guide comprises (i) a spacer sequence that is substantially complementary to a target sequence within a TRAC gene and (ii) a direct repeat sequence.

In one aspect of the composition, the target sequence is within exon 1, exon 2, exon 3, or exon 4 of the TRAC gene.

In another aspect of the composition, the TRAC gene comprises the sequence of SEQ ID NO: 339, the reverse complement of SEQ ID NO: 339, a variant of the sequence of SEQ ID NO: 339, or the reverse complement of a variant of SEQ ID NO: 339.

In another aspect of the composition, the spacer sequence comprises: a. nucleotide 1 through nucleotide 16 of a sequence that is at least 90% identical to a sequence of any one of SEQ ID NOs: 174-336; b. nucleotide 1 through nucleotide 17 of a sequence that is at cast 90% identical to a sequence of any one of SEQ ID NOs: 174-336; c. nucleotide 1 through nucleotide 18 of a sequence that is at least 90% identical to a sequence of any one of SEQ ID NOs: 174-336; d. nucleotide 1 through nucleotide 19 of a sequence that is at least 90% identical to a sequence of any one of SEQ ID NOs: 174-336; e. nucleotide 1 through nucleotide 20 of a sequence that is at least 90% identical to a sequence of any one of SEQ ID NOs: 174-336; f. nucleotide 1 through nucleotide 21 of a sequence that is at least 90% identical to a sequence of any one of SEQ ID NOs: 174-336; g. nucleotide 1 through nucleotide 22 of a sequence that is at least 90% identical to a sequence of any one of SEQ ID NOs: 174-336; h. nucleotide 1 through nucleotide 23 of a sequence that is at least 90% identical to a sequence of any one of SEQ ID NOs: 174-336; i. nucleotide 1 through nucleotide 24 of a sequence that is at least 90% identical to a sequence of any one of SEQ ID NOs: 174-336; j. nucleotide 1 through nucleotide 25 of a sequence that is at least 90% identical to a sequence of any one of SEQ ID NOs: 174-275 and 277-336; k. nucleotide 1 through nucleotide 26 of a sequence that is at least 90% identical to a sequence of any one of SEQ ID NOs: 174-275 and 277-336; l. nucleotide 1 through nucleotide 27 of a sequence that is at least 90% identical to a sequence of any one of SEQ ID NOs: 174-275 and 277-336; m. nucleotide 1 through nucleotide 28 of a sequence that is at least 90% identical to a sequence of any one of SEQ ID NOs: 174-275 and 277-336; n. nucleotide 1 through nucleotide 29 of a sequence that is at least 90% identical to a sequence of any one of SEQ ID NOs: 174-275 and 277-336; or o. nucleotide 1 through nucleotide 30 of a sequence that is at least 90% identical to a sequence of any one of SEQ ID NOs: 174-275 and 277-336.

In another aspect of the composition, the spacer sequence comprises: a. nucleotide 1 through nucleotide 16 of any one of SEQ ID NOs: 174-336; h. nucleotide 1 through nucleotide 17 of any one of SEQ ID NOs: 174-336; c. nucleotide 1 through nucleotide 18 of any one of SEQ ID NOs: 174-336; d. nucleotide 1 through nucleotide 19 of any one of SEQ ID NOs: 174-336; e. nucleotide 1 through nucleotide 20 of any one of SEQ ID NOs: 174-336; f. nucleotide 1 through nucleotide 21 of any one of SEQ ID NOs: 174-336; g. nucleotide 1 through nucleotide 22 of any one of SEQ ID NOs: 174-336; h. nucleotide 1 through nucleotide 23 of any one of SEQ ID NOs: 174-336; i. nucleotide 1 through nucleotide 24 of any one of SEQ ID NOs: 174-336; j. nucleotide 1 through nucleotide 25 of any one of SEQ ID NOs: 174-275 and 277-336; k. nucleotide 1 through nucleotide 26 of any one of SEQ ID NOs: 174-275 and 277-336; l. nucleotide 1 through nucleotide 27 of any one of SEQ ID NOs: 174-275 and 277-336; m. nucleotide 1 through nucleotide 28 of any one of SEQ ID NOs: 174-275 and 277-336; n. nucleotide 1 through nucleotide 29 of any one of SEQ ID NOs: 174-275 and 277-336; or o. nucleotide 1 through nucleotide 30 of any one of SEQ ID NOs: 174-275 and 277-336.

In another aspect of the composition, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8: d. nucleotide 4 through nucleotide 36 of a sequence that is at least 90% identical to a sequence of any one of SEQ ID NOs: 1-8; c. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8; g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8; j. nucleotide 10 through nucleotide 36 of a sequence that is at least 90% identical to a sequence of any one of SEQ ID NOs: 1-8: k. nucleotide 11 through nucleotide 36 of a sequence that is at least 90% identical to a sequence of any one of SEQ ID NOs: 1-8; l. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 though nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 1 i through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In another aspect of the composition, the direct repeat comprises: a. nucleotide 1 through nucleotide 36 of any one of SEQ ID NOs: 1-8; b. nucleotide 2 through nucleotide 36 of any one of SEQ ID NOs: 1-8; c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8: h. nucleotide 8 through nucleotide 36 of any one of SEQ ID NOs: 1-8: i. nucleotide 9 through nucleotide 36 of any one of SEQ ID NOs: 1-8; j. nucleotide 10 through nucleotide 36 of any one of SEQ ID NOs: 1-8; k. nucleotide 11 through nucleotide 36 of any one of SEQ ID NOs: 1-8; I. nucleotide 12 through nucleotide 36 of any one of SEQ ID NOs: 1-8; m. nucleotide 13 through nucleotide 36 of any one of SEQ ID NOs: 1-8; n. nucleotide 14 through nucleotide 36 of any one of SEQ ID NOs: 1-8; o. nucleotide 1 through nucleotide 34 of SEQ ID NO: 9: p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9; t. nucleotide 6 through nucleotide 34 of SEQ ID NO: 9: n. nucleotide 7 through nucleotide 34 of SEQ ID NO: 9; v nucleotide 8 through nucleotide 34 of SEQ ID NO: 9; w. nucleotide 9 through nucleotide 34 of SEQ ID NO: 9; x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9: or aa. SEQ ID NO: 10 or a portion thereof.

In another aspect of the composition, the direct repeat comprises: a. nucleotide 1 through nucleotide 36 of a sequence that is at least 90% identical to a sequence of any one of SEQ ID NOs: 359-376; b. nucleotide 2 through nucleotide 36 of a sequence that is at least 90% identical to a sequence of any one of SEQ ID NOs: 359-376; c. nucleotide 3 through nucleotide 36 of a sequence that is at least 90% identical to a sequence of any one of SEQ ID NOs: 359-376; d nucleotide 4 through nucleotide 36 of a sequence that is at least 90% identical to a sequence of any one of SEQ ID NOs: 359-376; e. nucleotide 5 through nucleotide 36 of a sequence that is at least 90% identical to a sequence of any one of SEQ ID NOs: 359-376; f. nucleotide 6 through nucleotide 36 of a sequence that is at least 90% identical to a sequence of any one of SEQ ID NOs: 359-376; g. nucleotide 7 through nucleotide 36 of a sequence that is at least 90% identical to a sequence of any one of SEQ ID NOs: 359-376; h. nucleotide 8 through nucleotide 36 of a sequence that is at least 90% identical to a sequence of any one of SEQ ID NOs: 359-376; i. nucleotide 9 through nucleotide 36 of a sequence that is at least 90% identical to a sequence of any one of SEQ ID NOs: 359-376: j. nucleotide 10 through nucleotide 36 of a sequence that is at least 90% identical to a sequence of any one of SEQ ID NOs: 359-376; k. nucleotide 11 through nucleotide 36 of a sequence that is at least 90% identical to a sequence of any one of SEQ ID NOs: 359-376; l. nucleotide 12 through nucleotide 36 of a sequence that is at least 90% identical to a sequence of any one of SEQ ID NOs: 359-376; m. nucleotide 13 through nucleotide 36 of a sequence that is at least 90% identical to a sequence of any one of SEQ ID NOs: 359-376; n. nucleotide 14 through nucleotide 36 of a sequence that is at least 90% identical to a sequence of any one of SEQ ID NOs: 359-376; or o. a sequence that is at least 90% identical to a sequence of SEQ ID NO: 377 or a portion thereof.

In another aspect of the composition, the direct repeat comprises: a. nucleotide 1 through nucleotide 36 of any one of SEQ ID NOs: 359-376; h. nucleotide 2 through nucleotide 36 of any one of SEQ ID NOs: 359-376; c. nucleotide 3 through nucleotide 36 of any one of SEQ ID NOs: 359-376; d. nucleotide 4 through nucleotide 36 of any one of SEQ ID NOs: 359-376; e. nucleotide 5 through nucleotide 36 of any one of SEQ ID NOs: 359-376; f. nucleotide 6 through nucleotide 36 of any one of SEQ ID NOs: 359-376; g. nucleotide 7 through nucleotide 36 of any one of SEQ ID NOs: 359-376; h. nucleotide 8 through nucleotide 36 of any one of SEQ ID NOs: 359-376; i. nucleotide 9 through nucleotide 36 of any one of SEQ ID NOs: 359-376; j. nucleotide 10 through nucleotide 36 of any one of SEQ ID NOs: 359-376; k. nucleotide 11 through nucleotide 36 of any one of SEQ ID NOs: 359-376; l. nucleotide 12 through nucleotide 36 of any one of SEQ ID NOs: 359-376; n. nucleotide 13 through nucleotide 36 of any one of SEQ ID NOs: 359-376; n. nucleotide 14 through nucleotide 36 of any one of SEQ ID NOs: 359-376: or o. SEQ ID NO: 377 or a portion thereof.

In another aspect of the composition, the dire repeat comprises: a nucleotide 1 through nucleotide 36 of a sequence that is at least 90% identical to SEQ HD NO: 378; b. nucleotide 2 through nucleotide 36 of a sequence that is at least 90% identical to SEQ ID NO: 378; c. nucleotide 3 through nucleotide 36 of a sequence that is at least 90% identical to SEQ ID NO: 378; d. nucleotide 4 through nucleotide 36 of a sequence that is at least 90% identical to SEQ ID NO: 378; e. nucleotide 5 through nucleotide 36 of a sequence that is at least 90% identical to SEQ ID NO: 378; f. nucleotide 6 through nucleotide 36 of a sequence that is at least 90% identical to SEQ ID NO: 378; g. nucleotide 7 through nucleotide 36 of a sequence that is at least 90% identical to SEQ ID NO: 378; h. nucleotide 8 through nucleotide 36 of a sequence that is at least 90% identical to SEQ ID NO: 378; i. nucleotide 9 through nucleotide 36 of a sequence that is at least 90% identical to SEQ ID NO: 378; j. nucleotide 10 through nucleotide 36 of a sequence that is at least 90% identical to SEQ ID NO: 378; k. nucleotide 11 through nucleotide 36 of a sequence that is at least 90% identical to SEQ ID NO: 378; l. nucleotide 12 through nucleotide 36 of a sequence that is at least 90% identical to SEQ ID NO: 378; m. nucleotide 13 through nucleotide 36 of a sequence that is at least 90% identical to SEQ ID NO: 378; n. nucleotide 14 through nucleotide 36 of a sequence that is at least 90% identical to SEQ ID NO: 378; or o. a sequence that is at least 90% identical to a sequence of SEQ ID NO: 379 or SEQ ID NO: 380 or a portion thereof.

In another aspect of the composition, the direct repeat comprises: a. nucleotide 1 through nucleotide 36 of SEQ ID NO: 378; h. nucleotide 2 through nucleotide 36 of SEQ ID NO: 378; c. nucleotide 3 through nucleotide 36 of SEQ ID NO: 378; d. nucleotide 4 through nucleotide 36 of SEQ ID NO: 378; e. nucleotide 5 through nucleotide 36 of SEQ ID NO: 378; f. nucleotide 6 through nucleotide 36 of SEQ ID NO: 378; g. nucleotide 7 through nucleotide 36 of SEQ ID NO: 378; h. nucleotide 8 through nucleotide 36 of SEQ ID NO: 378; i. nucleotide 9 through nucleotide 36 of SEQ ID NO: 378; j. nucleotide 10 through nucleotide 36 of SEQ ID NO: 378; k. nucleotide 11 through nucleotide 36 of SEQ ID NO: 378; l. nucleotide 12 through nucleotide 36 of SEQ ID NO: 378; m. nucleotide 13 through nucleotide 36 of SEQ ID NO: 378; n. nucleotide 14 through nucleotide 36 of SEQ ID NO: 378; or o. SEQ ID NO: 379 or SEQ ID NO: 380 or a portion thereof.

In another aspect of the composition, the direct repeat comprises: a. nucleotide 1 through nucleotide 36 of a sequence that is at least 90% identical to a sequence of SEQ ID NO: 381 or SEQ ID NO: 382; b. nucleotide 2 through nucleotide 36 of a sequence that is at least 90% identical to a sequence of SEQ ID NO: 381 or SEQ ID NO: 382; c. nucleotide 3 through nucleotide 36 of a sequence that is at least 90% identical to a sequence of SEQ ID NO: 381 or SEQ ID NO: 382: d. nucleotide 4 through nucleotide 36 of a sequence that is at least 90% identical to a sequence of SEQ ID NO: 381 or SEQ ID NO: 382; e. nucleotide 5 through nucleotide 36 of a sequence that is at least 90% identical to a sequence of SEQ ID NO: 381 or SEQ ID NO: 382; f. nucleotide 6 through nucleotide 36 of a sequence that is at least 90% identical to a sequence of SEQ ID NO: 381 or SEQ ID NO: 382; g. nucleotide 7 through nucleotide 36 of a sequence that is at least 90% identical to a sequence of SEQ ID NO: 381 or SEQ ID NO: 382; h. nucleotide 8 through nucleotide 36 of a sequence that is at least 90% identical to a sequence of SEQ ID NO: 381 or SEQ ID NO: 382: i. nucleotide 9 through nucleotide 36 of a sequence that is at least 90% identical to a sequence of SEQ ID NO: 381 or SEQ ID NO: 382; j. nucleotide 10 through nucleotide 36 of a sequence that is at least 90% identical to a sequence of SEQ ID NO: 381 or SEQ ID NO: 382: k. nucleotide 11 through nucleotide 36 of a sequence that is at least 90% identical to a sequence of SEQ ID NO: 381 or SEQ ID NO: 382; l. nucleotide 12 through nucleotide 36 of a sequence that is at least 90% identical to a sequence of SEQ ID NO: 381 or SEQ ID NO: 382; m. nucleotide 13 through nucleotide 36 of a sequence that is at least 90% identical to a sequence of SEQ ID NO: 381 or SEQ ID NO: 382; n. nucleotide 14 through nucleotide 36 of a sequence that is at least 90% identical to a sequence of SEQ ID NO: 381 or SEQ ID NO: 382; o. nucleotide 15 through nucleotide 36 of a sequence that is at least 90% identical to a sequence of SEQ ID NO: 381 or SEQ ID NO: 382; or p. a sequence that is at lea-t 90% identical to a sequence of SEQ ID NO: 383 or a portion thereof.

In another aspect of the composition, the direct repeat comprises: a. nucleotide 1 through nucleotide 36 of SEQ ID NO: 381 or SEQ ID NO: 382; b. nucleotide 2 through nucleotide 36 of SEQ ID NO: 381 or SEQ ID NO: 382; c. nucleotide 3 through nucleotide 36 of SEQ ID NO: 381 or SEQ ID NO: 382; d. nucleotide 4 through nucleotide 36 of SEQ ID NO: 381 or SEQ ID NO: 382; e. nucleotide 5 through nucleotide 36 of SEQ ID NO: 381 or SEQ ID NO: 382; f. nucleotide 6 through nucleotide 36 of SEQ ID NO: 381 or SEQ ID NO: 382; g. nucleotide 7 through nucleotide 36 of SEQ ID NO: 381 or SEQ ID NO: 382; h. nucleotide 8 through nucleotide 36 of SEQ ID NO: 381 or SEQ ID NO: 382: i. nucleotide 9 through nucleotide 36 of SEQ ID NO: 381 or SEQ ID NO: 382; j. nucleotide 10 through nucleotide 36 of SEQ ID NO: 381 or SEQ ID NO: 382; k. nucleotide 11 through nucleotide 36 of SEQ ID NO: 381 or SEQ ID NO: 382; l. nucleotide 12 through nucleotide 36 of SEQ ID NO: 381 or SEQ ID NO: 382; m. nucleotide 13 through nucleotide 36 of SEQ ID NO: 381 or SEQ ID NO: 382; n. nucleotide 14 through nucleotide 36 of SEQ ID NO: 381 or SEQ ID NO: 382; o. nucleotide 15 through nucleotide 36 of SEQ ID NO: 381 or SEQ ID NO: 382; or p. SEQ ID NO: 383 or a portion thereof.

In another aspect of the composition, the spacer sequence is substantially complementary to the complement of a sequence of any one of SEQ ID NOs: 11-173.

In another aspect of the composition, the target sequence is adjacent to a protospacer adjacent motif (PAM) comprising the sequence 5′-NTTN-3′.

In another aspect of the composition, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In another aspect of the composition, the target sequence is immediately adjacent to the PAM sequence.

In another aspect of the composition, the target sequence is within 1, 2, 3, 4, or 5 nucleotides of the PAM sequence.

In another aspect of the composition, the Cas12i polypeptide is: (a) a Cas12i2 polypeptide comprising a sequence that is at least 90% identical to the sequence of SEQ ID NO: 338, SEQ ID NO: 348, SEQ ID NO: 349, SEQ ID NO: 350, SEQ ID NO: 351, or SEQ ID NO: 352; (b) a Cas12i4 polypeptide comprising a sequence that is at least 90% identical to the sequence of SEQ ID NO: 354, SEQ ID NO: 355, or SEQ ID NO: 356; (c) a Cas12i1 polypeptide comprising a sequence that is at least 90% identical to the sequence of SEQ ID NO: 357: or (d) a Cas12i3 polypeptide comprising a sequence that is at least 90% identical to the sequence of SEQ ID NO: 358.

In another aspect of the composition, the Cas12i polypeptide is: (a) a Cas12i2 polypeptide comprising a sequence of SEQ ID NO: 338, SEQ ID NO: 348, SEQ ID NO: 349, SEQ ID NO: 350, SEQ ID NO: 351, or SEQ ID NO: 352; (b) a Cas12i4 polypeptide comprising a sequence of SEQ ID NO: 354, SEQ ID NO: 355, or SEQ ID NO: 356; (c) a Cas12i1 polypeptide comprising a sequence of SEQ ID NO: 357; or (d) a Cas12i3 polypeptide comprising a sequence of SEQ ID NO: 358.

In another aspect of the composition, the RNA guide and the Cas12i polypeptide form a ribonucleoprotein complex.

In another aspect of the composition, the ribonucleoprotein complex binds a target nucleic acid.

In another aspect of the composition, the composition is present within a cell.

In another aspect of the composition, the RNA guide and the Cas12i polypeptide are encoded in a vector, e.g., expression vector. In another aspect of the composition, the RNA guide and the Cas12i polypeptide are encoded in a single vector or the RNA guide is encoded in a first vector and the Cas12i polypeptide is encoded in a second vector.

The invention further provides a vector system comprising one or more vectors encoding an RNA guide disclosed herein and a Cas12i polypeptide. In an embodiment, the vector system comprises a first vector encoding an RNA guide disclosed herein and a second vector encoding a Cas12i polypeptide. The vectors may be expression vectors.

In another aspect of the composition, the RNA guide does not consist of the sequence of:

(SEQ ID NO: 344)

AGAAAUCCGUCUUUCAUUGACGGAAGAGCAACAGUGCUGUGGC;

(SEQ ID NO: 345)

AGAAAUCCGUCUUUCAUUGACGGAACAACAGCAUUAUUCCAGA;

(SEQ ID NO: 346)

AGAAAUCCGUCUUUCAUUGACGGGAAACAGGUAAGACAGGGGU;
or

(SEQ ID NO: 347)

AGAAAUCCGUCUUUCAUUGACGGCAGGAGGAGGAUUCGGAACC.

The invention yet further provides an RNA guide comprising (i) a spacer sequence that is substantially complementary to a target sequence within a TRAC gene and (ii) a direct repeat sequence.

In one aspect of the RNA guide, the target sequence is within exon 1. exon 2. exon 3, or exon 4 of the TRAC gene.

In another aspect of the RNA guide, the TRAC gene comprises the sequence of SEQ ID NO: 339, the reverse complement of SEQ ID NO: 339, a variant of the sequence of SEQ ID NO: 339, or the reverse complement of a variant of SEQ ID NO: 339.

In another aspect of the RNA guide, the spacer sequence comprises: a. nucleotide 1 through nucleotide 16 of a sequence that is at least 90% identical to a sequence of any one of SEQ ID NOs: 174-336; b. nucleotide 1 through nucleotide 17 of a sequence that is at least 90% identical to a sequence of any one of SEQ ID NOs: 174-336; c. nucleotide 1 through nucleotide 18 of a sequence that is at least 90% identical to a sequence of any one of SEQ ID NOs: 174-336; d. nucleotide 1 through nucleotide 19 of a sequence that is at least 90% identical to a sequence of any one of SEQ ID NOs: 174-336; e. nucleotide 1 through nucleotide 20 of a sequence that is at least 90% identical to a sequence of any one of SEQ ID NOs: 174-336; f. nucleotide 1 through nucleotide 21 of a sequence that is at least 90% identical to a sequence of any one of SEQ ID NOs: 174-336; g. nucleotide 1 through nucleotide 22 of a sequence that is at least 90% identical to a sequence of any one of SEQ ID NOs: 174-336; h. nucleotide 1 through nucleotide 23 of a sequence that is at least 90% identical to a sequence of any one of SEQ ID NOs: 174-336; i. nucleotide 1 through nucleotide 24 of a sequence that is at least 90% identical to a sequence of any one of SEQ ID NOs: 174-336; j. nucleotide 1 through nucleotide 25 of a sequence that is at least 90% identical to a sequence of any one of SEQ ID NOs: 174-275 and 277-336; k. nucleotide 1 through nucleotide 26 of a sequence that is at least 90% identical to a sequence of any one of SEQ ID NOs: 174-275 and 277-336; l. nucleotide 1 through nucleotide 27 of a sequence that is at least 90% identical to a sequence of any one of SEQ ID NOs: 174-275 and 277-336; m. nucleotide 1 through nucleotide 28 of a sequence that is at least 90% identical to a sequence of any one of SEQ ID NOs: 174-275 and 277-336; n. nucleotide 1 through nucleotide 29 of a sequence that is at least 90% identical to a sequence of any one of SEQ ID NOs: 174-275 and 277-336; or o. nucleotide 1 through nucleotide 30 of a sequence that is at least 90% identical to a sequence of any one of SEQ ID NOs: 174-275 and 277-336.

In another aspect of the composition, the spacer sequence comprises: a. nucleotide 1 through nucleotide 16 of any one of SEQ ID NOs: 174-336; b. nucleotide 1 through nucleotide 17 of any one of SEQ ID NOs: 174-336; c. nucleotide 1 through nucleotide 18 of any one of SEQ ID NOs: 174-336; d. nucleotide 1 through nucleotide 19 of any one of SEQ ID NOs: 174-336; c. nucleotide 1 through nucleotide 20 of any one of SEQ ID NOs: 174-336; f. nucleotide 1 through nucleotide 21 of any one of SEQ ID NOs: 174-336; g. nucleotide 1 through nucleotide 22 of any one of SEQ 10) NOs: 174-336; h. nucleotide 1 through nucleotide 23 of any one of SEQ ID NOs: 174-336: i. nucleotide 1 through nucleotide 24 of any one of SEQ ID NOs: 174-336; j. nucleotide 1 through nucleotide 25 of any one of SEQ ID NOs: 174-275 and 277-336; k. nucleotide 1 through nucleotide 26 of any one of SEQ ID NOs: 174-275 and 277-336; l. nucleotide 1 through nucleotide 27 of any one of SEQ ID NOs: 174-275 and 277-336; m. nucleotide 1 through nucleotide 28 of any one of SEQ ID NOs: 174-275 and 277-336; n. nucleotide 1 through nucleotide 29 of any one of SEQ ID NOs: 174-275 and 277-336; or o nucleotide 1 through nucleotide 30 of any one of SEQ ID NOs: 174-275 and 277-336.

In another aspect of the RNA guide, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8; d. nucleotide 4 through nucleotide 36 of a sequence that is at least 90% identical to a sequence of any one of SEQ ID NOs: 1-8; e. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8; g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8; j. nucleotide 10 through nucleotide 36 of a sequence that is at least 90% identical to a sequence of any one of SEQ ID NOs: 1-8; k. nucleotide 11 through nucleotide 36 of a sequence that is at least 90% identical to a sequence of any one of SEQ ID NOs: 1-8; l. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 through nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 11 through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In another aspect of the RNA guide, the direct repeat comprises: a. nucleotide 1 through nucleotide 36 of any one of SEQ ID NOs: 1-8; b. nucleotide 2 through nucleotide 36 of any one of SEQ ID NOs: 1-8; c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8; h. nucleotide 8 through nucleotide 36 of any one of SEQ ID NOs: 1-8; i. nucleotide 9 through nucleotide 36 of any one of SEQ ID NOs: 1-8; j. nucleotide 10 through nucleotide 36 of any one of SEQ ID NOs: 1-8: k. nucleotide 11 through nucleotide 36 of any one of SEQ ID NOs: 1-8; l. nucleotide 12 through nucleotide 36 of any one of SEQ ID NOs: 1-8; m. nucleotide 13 through nucleotide 36 of any one of SEQ ID NO: 1-8; n. nucleotide 14 through nucleotide 36 of any one of SEQ ID NOs: 1-8; o. nucleotide 1 through nucleotide 34 of SEQ ID NO: 9; p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9; t. nucleotide 6 through nucleotide 34 of SEQ ID NO: 9; u. nucleotide 7 through nucleotide 34 of SEQ ID NO: 9; v. nucleotide 8 through nucleotide 34 of SEQ ID NO: 9; w. nucleotide 9 through nucleotide 34 of SEQ ID NO: 9; x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9; or aa. SEQ ID NO: 10 or a portion thereof.

In another aspect of the RNA guide, the spacer sequence is substantially complementary to the complement of a sequence of any one of SEQ ID NOs: 11-173.

In another aspect of the RNA guide, the target sequence is adjacent to a protospacer adjacent motif (PAM) comprising the sequence 5′-NTTN-3′, wherein N is any nucleotide.

In another aspect of the RNA guide, the RNA guide does not consist of the sequence of:

(SEQ ID NO: 344)

AGAAAUCCGUCUUUCAUUGACGGAAGAGCAACAGUGCUGUGGC;

(SEQ ID NO: 345)

AGAAAUCCGUCUUUCAUUGACGGAACAACAGCAUUAUUCCAGA;

(SEQ ID NO: 346)

AGAAAUCCGUCUUUCAUUGACGGGAAACAGGUAAGACAGGGGU;
or

(SEQ ID NO: 347)

AGAAAUCCGUCUUUCAUUGACGGCAGGAGGAGGAUUCGGAACC.

In another aspect of the RNA guide, the PAM comprises the sequence 5′-AT′A-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTG-3′, 5′-GTTC-3′, 5′-CTTA-3′, 5′-CTTG-3′, 5′-CTTG-3′, or 5′-CTC-3′.

In another aspect of the RNA guide, the target sequence is immediately adjacent to the PAM sequence.

In another aspect of the RNA guide, the target sequence is within 1, 2, 3, 4, or 5 nucleotides of the PAM sequence.

The invention yet further provides a nucleic acid encoding an RNA guide as described herein.

The invention yet further provides a vector comprising such an RNA guide as described herein.

The invention yet further provides a cell comprising a composition, an RNA guide, a nucleic acid, or a vector as described herein.

In one aspect of the cell, the cell is a eukaryotic cell, an animal cell, a mammalian cell, a human cell, a primary cell, a cell line, a stem cell, or a T cell.

The invention yet further provides a kit comprising a composition, an RNA guide, a nucleic acid, or a vector as described herein.

The invention yet further provides a method of editing a TRAC sequence, the method comprising contacting a TRAC sequence with a composition or an RNA guide as described herein. In an embodiment. the method is carried out in vitro. In an embodiment, the method is carried out ex vivo.

In one aspect of the method, the TRAC sequence is in a cell.

In one aspect of the method, the composition or the RNA guide induces a deletion in the TRAC sequence.

In one aspect of the method, the deletion is adjacent to a 5′-NTTN-3′ sequence, wherein N is any nucleotide.

In one aspect of the method, the deletion is downstream of the 5′-NTTN-3′ sequence.

In one aspect of the method, the deletion is up to about 40 nucleotides in length.

In one aspect of the method, the deletion is from about 4 nucleotides to 40 nucleotides in length.

In one aspect of the method, the deletion is from about 4 nucleotides to 25 nucleotides in length.

In one aspect of the method, the deletion is from about 10 nucleotides to 25 nucleotides in length.

In one aspect of the method, the deletion is from about 10 nucleotides to 15 nucleotides in length.

In one aspect of the method, the deletion starts within about 5 nucleotides to about 15 nucleotides of the 5′-NTTN-3′ sequence.

In one aspect of the method, the deletion starts within about 5 nucleotides to about 10 nucleotides of the 5′-NTTN-3′ sequence.

In one aspect of the method, the deletion starts within about 10 nucleotides to about 15 nucleotides of the 5′-NTTN-3′ sequence.

In one aspect of the method, the deletion starts within about 5 nucleotides to about 15 nucleotides downstream of the 5′-NTTN-3′ sequence.

In one aspect of the method, the deletion starts within about 5 nucleotides to about 10 nucleotides downstream of the 5′-NTTN-3′ sequence.

In one aspect of the method, the deletion starts within about 10 nucleotides to about 15 nucleotides downstream of the 5′-NTTN-3′ sequence.

In one aspect of the method, the deletion ends within about 20 nucleotides to about 30 nucleotides of the 5′-N1′N-3′ sequence.

In one aspect of the method, the deletion ends within about 20 nucleotides to about 25 nucleotides of the 5′-NTTN-3′ sequence.

In one aspect of the method, the deletion ends within about 25 nucleotides to about 30 nucleotides of the 5′-NTTN-3′ sequence.

In one aspect of the method, the deletion ends within about 20 nucleotides to about 30 nucleotides downstream of the 5′-NTTN-3′ sequence.

In one aspect of the method, the deletion ends within about 20 nucleotides to about 25 nucleotides downstream of the 5′-NTTN-3′ sequence.

In one aspect of the method, the deletion ends within about 25 nucleotides to about 30 nucleotides downstream of the 5′-NTTN-3′ sequence.

In one aspect of the method, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In one aspect of the method, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In one aspect of the method, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In one aspect of the method, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In one aspect of the method, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In one aspect of the method, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In one aspect of the method, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In one aspect of the method, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In one aspect of the method, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In one aspect of the method, the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

In one aspect of the method, the deletion overlaps with a mutation in the TRAC sequence.

In one aspect of the method, the deletion overlaps with an insertion in the TRAC sequence.

In one aspect of the method, the deletion removes a repeat expansion of the TRAC sequence or a portion thereof.

In one aspect of the method, the deletion disrupts one or both alleles of the TRAC sequence.

In one aspect of the composition, RNA guide, nucleic acid, vector, cell, kit, or method described herein, the RNA guide does not consist of the sequence of:

(SEQ ID NO: 344)

AGAAAUCCGUCUUUCAUUGACGGAAGAGCAACAGUGCUGUGGC;

(SEQ ID NO: 345)

AGAAAUCCGUCUUUCAUUGACGGAACAACAGCAUUAUUCCAGA;

(SEQ ID NO: 346)

AGAAAUCCGUCUUUCAUUGACGGGAAACAGGUAAGACAGGGGU;
or

(SEQ ID NO: 347)

AGAAAUCCGUCUUUCAUUGACGGCAGGAGGAGGAUUCGGAACC.

In one aspect of the composition, RNA guide, nucleic acid, vector, cell, kit, or method described herein, the RNA guide comprises the sequence of any one of SEQ ID NOs: 385-397.

### Definitions

The present invention will be described with respect to particular embodiments, but the invention is not limited thereto but only by the claims. Terms as set forth hereinafter are generally to be understood in their common sense unless indicated otherwise.

As used herein, the term “activity” refers to a biological activity. In some embodiments, activity includes enzymatic activity, e.g., catalytic ability of an effector. For example, activity can include nuclease activity

As used herein, the term “Cas12i polypeptide” (also referred to herein as Cas12i) refers to a polypeptide that binds to a target sequence on a target nucleic acid specified by an RNA guide, wherein the polypeptide has at least some amino acid sequence homology to a wild-type Cas12i polypeptide. In some embodiments, the Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 1-5 and 11-18 of U.S. Pat. No. 10,808,245, which is incorporated by reference herein in its entirety. In some embodiments, a Cas12i polypeptide comprises at least 75%, at least 80%, at least 81%. at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NO: 3 (Cas12i1), SEQ ID NO: 5 (Cas12i2), SEQ ID NO: 14 (Cas12i3), or SEQ ID NO: 16 (Cas12i4) of U.S. Pat. No. 10,808,245, corresponding to SEQ ID NOs: 357, 338, 358, and 354 of the present application. In some embodiments, a Cas12i polypeptide of the disclosure is a Cas12i1 polypeptide or Cas12i2 polypeptide as described in PCT/US2021/025257. In some embodiments, the Cas12i polypeptide cleaves a target nucleic acid (e.g., as a nick or a double strand break).

As used herein, the term “complex” refers to a grouping of two or more molecules. In some embodiments, the complex comprises a polypeptide and a nucleic acid molecule interacting with (e.g., binding to, coming into contact with, adhering to) one another. As used herein, the term “complex” can refer to a grouping of an RNA guide and a polypeptide (e.g., a Cas12i polypeptide). As used herein, the term “complex” can refer to a grouping of an RNA guide, a polypeptide, and a target sequence. As used herein, the term “complex” can refer to a grouping of a TRAC-targeting RNA guide and a Cas12i polypeptide.

As used herein, the term “protospacer adjacent motif” or “PAM” refers to a DNA sequence adjacent to a target sequence (e.g., a TRAC target sequence) to which a complex comprising an RNA guide (e.g., a TRAC-targeting RNA guide) and a Cas12i polypeptide binds. In the case of a double-stranded target, the RNA guide hinds to a first strand of the target (e.g., the target strand or the spacer-complementary strand), and a PAM sequence as described herein is present in the second, complementary strand (e.g., the non-target strand or the non-spacer-complementary strand). As used herein, the term “adjacent” includes instances in which the RNA guide of a complex comprising an RNA guide and a Cas12i polypeptide specifically binds, interacts, or associates with a target sequence that is immediately adjacent to a PAM. In such instances, there are no nucleotides between the target sequence and the PAM. The term “adjacent” also includes instances in which there are a small number (e.g., 1, 2, 3, 4, or 5) of nucleotides between the target sequence, to which the RNA guide binds, and the PAM. In some embodiments, the PAM sequence as described herein is present in the non-target strand (e.g., the non-spacer-complementary strand). In such a case, the term “adjacent” includes a PAM sequence as described herein as being immediately adjacent to (or within a small number, e.g., 1, 2, 3, 4, or 5 nucleotides of) a sequence in the non-target strand.

As used herein, the term “RNA guide” refers to any RNA molecule that facilitates the targeting of a polypeptide (e.g., a Cas12i polypeptide) described herein to a target sequence (e.g., a sequence of a TRAC gene). An RNA guide may be designed to include sequences that are complementary to a specific nucleic acid sequence (e.g., a TRAC nucleic acid sequence). An RNA guide may comprise a DNA targeting sequence (i.e., a spacer sequence) and a direct repeat (DR) sequence. The term “crRNA” is also used herein to refer to an RNA guide.

In some embodiments, a spacer sequence is complementary to a target sequence. As used hemin, the term “complementary” refers to the ability of nucleobases of a first nucleic acid molecule, such as an RNA guide, to base pair with nucleobases of a second nucleic acid molecule, such as a target sequence. Two complementary nucleic acid molecules are able to non-covalently bind under appropriate temperature and solution ionic strength conditions. In some embodiments, a first nucleic acid molecule (e.g., a spacer sequence of an RNA guide) comprises 100% complementarity to a second nucleic acid (e.g., a target sequence). In some embodiments, a first nucleic acid molecule (e.g., a spacer sequence of an RNA guide) is complementary to a second nucleic acid molecule (e.g., a target sequence) if the first nucleic acid molecule comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid. As used herein, the term “substantially complementary” refers to a polynucleotide (e.g., a spacer sequence of an RNA guide) that has a certain level of complementarity to a target sequence. In some embodiments, the level of complementarity is such that the polynucleotide can hybridize to the target sequence with sufficient affinity to permit an effector polypeptide (e.g., Cas12i) that is complexed with the polynucleotide to act (e.g., cleave) on the target sequence. In some embodiments, a spacer sequence that is substantially complementary to a target sequence has less than 100% complementarity to the target sequence. In some embodiments, a spacer sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence. In some embodiments, an RNA guide with a spacer sequence that is substantially complementary to a target sequence has 100% complementary to the target sequence.

As used herein, the terms “target” and “target sequence” refer to a nucleic acid sequence to which an RNA guide specifically binds. In some embodiments, the DNA targeting sequence (e.g., spacer) of an RNA guide binds to a target sequence. In the case of a double-stranded target, the RNA guide binds to a first strand of the target (i.e., the target strand or the spacer-complementary strand), and a PAM sequence as described herein is present in the second, complementary strand (i.e., the non-target strand or the non-spacer-complementary strand). In some embodiments, the target strand (i.e., the spacer-complementary strand) comprises a 5′-NAAN-3′ sequence. In some embodiments, the target sequence is a sequence within a TRAC gene sequence, including, but not limited, to the sequence set forth in SEQ ID NO: 339 or the reverse complement thereof.

As used herein the term “TRAC” refers to “T cell receptor alpha chain constant.” TRAC, together with the T cell receptor alpha chain variable region, comprises the T cell receptor alpha chain component of the T cell receptor protein complex found on the surface of T lymphocytes. SEQ ID NO: 339 as set forth herein provides an example of a TRAC gene sequence. It is understood that spacer sequences described herein can target SEQ ID NO: 339 or the reverse complement thereof, depending upon whether they are indicated as “+” or “−” am set forth in Table 5. The target sequences listed in Table 5 and Table 6 are on the non-target strand of the TRAC gene.

As used herein, the terms “upstream” and “downstream” refer to relative positions within a single nucleic acid (e.g., DNA) sequence in a nucleic acid molecule. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the 5′-NTTN-3′ sequence is upstream of an indel described herein, and a Cas12i-induced indel is downstream of the 5′-N1′1N-3′ sequence.

### BRIEF DESCRIPTION OF THE DRAWINGS

FIG. 1 shows indel activity by variant Cas12i2 of SEQ ID NO: 348 and several individual RNA guides targeting TRAC at various concentrations in HEK293T cells.

FIG. 2 indel activity by variant Cas12i2 of SEQ ID NO: 349 and several individual RNA guides targeting TRAC at various concentrations in primary T cells. Error bars represent standard deviation of the mean of four technical replicates from one representative donor.

FIG. 3 shows viability of cells (via DAPI staining) seven days following introduction of variant Cas12i2 RNPs targeting TRAC at various concentrations in primary T cells. Error bars represent standard deviation of the mean of four technical replicates from one representative donor.

### DETAILED DESCRIPTION

The present disclosure relates to an RNA guide capable of binding to TRAC and methods of use thereof. In some aspects, a composition comprising an RNA guide having one or more characteristics is described herein. In some aspects, a method of producing the RNA guide is described. In some aspects, a method of delivering a composition comprising the RNA guide is described.

### Composition

In some aspects, the invention described herein comprises compositions comprising an RNA guide targeting TRAC. In some embodiments, the RNA guide is comprised of a direct repeat component and a pacer component. In some embodiments, the RNA guide hinds a Cas12i polypeptide. In some embodiments, the spacer component is substantially complementary to a TRAC target sequence, wherein the TRAC target sequence is adjacent to a 5′-NTTN-3′ PAM sequence as described herein. In the case of a double-stranded target, the RNA guide binds to a first strand of the target (i.e., the target strand or the spacer-complementary strand) and a PAM sequence as described herein is present in the second, complementary strand (i.e., the non-target strand or the non-spacer-complementary strand).

In some embodiments, the invention described herein comprises compositions comprising a complex, wherein the complex comprises an RNA guide targeting TRAC. In some embodiments, the invention comprises a complex comprising an RNA guide and a Cas12i polypeptide. In some embodiments, the RNA guide and the Cas12i polypeptide bind to each other in a molar ratio of about 1:1. In some embodiments, a complex comprising an RNA guide and a Cas12i polypeptide binds to a TRAC target sequence. In some embodiments, a complex comprising an RNA guide targeting TRAC and a Cas12i polypeptide binds to a TRAC target sequence at a molar ratio of about 1:1. In some embodiments, the complex comprises enzymatic activity, such as nuclease activity, that can cleave the TRAC target sequence. The RNA guide, the Cas12i polypeptide, and the TRAC target sequence, either alone or together, do not naturally occur.

Use of the compositions disclosed herein has advantages over those of other known nuclease systems. Cas12i polypeptides are smaller than other nucleases. For example, Cas12i2 is 1,054 amino acids in length, whereas S. pyogenes Cas9 (SpCas9) is 1,368 amino acids in length, S. thermophilus Cas9 (StCas9) is 1,128 amino acids in length, FnCpf1 is 1,300 amino acids in length, AsCpf1 is 1,307 amino acids in length, and LbCpf1 is 1,246 amino acids in length. Cas12i RNA guides, which do not require a trans-activating CRISPR RNA (tracrRNA), are also smaller than Cas9 RNA guides. The smaller Cas12i polypeptide and RNA guide sizes are beneficial for delivery. Compositions comprising a Cas12i polypeptide also demonstrate decreased off-target activity compared to compositions comprising an SpCas9 polypeptide. See PCT/US2021/025257, which is incorporated by reference in its entirety. Furthermore, indels induced by compositions comprising a Cas12i polypeptide differ from indels induced by compositions comprising an SpCas9 polypeptide. For example. SpCas9 polypeptides primarily induce insertions and deletions of 1 nucleotide in length. However, Cas12i polypeptides induce larger deletions, which cam be beneficial in disrupting a larger portion of a gene such as TRAC.

### RNA Guide

In some embodiments, the composition described herein comprises an RNA guide targeting a TRAC gene or a portion of a TRAC gene. In some embodiments, the composition described herein comprises two or more (e.g., 2, 3, 4, 5, 6, 7, 8, 9, or more) RNA guides targeting TRAC.

The RNA guide may direct the Cas12i polypeptide as described herein to a TRAC target sequence. Two or more RNA guides may target two or more separate Cas12i polypeptides (e.g., Cas12i polypeptides having the same or different sequence) as described herein to two or more (e.g., 2, 3, 4, 5, 6, 7, 8, 9, or more) TRAC target sequences.

Those skilled in the art reading the below examples of particular kinds of RNA guides will understand that, in some embodiments, an RNA guide is TRAC target-specific. That is, in some embodiments, an RNA guide binds specifically to one or more TRAC target sequences (e.g., within a cell) and not to non-targeted sequences (e.g., non-specific DNA or random sequences within the same cell).

In some embodiments, the RNA guide comprises a spacer sequence followed by a direct repeat sequence, referring to the sequences in the 5′ to 3′ direction. In some embodiments, the RNA guide comprises a first direct repeat sequence followed by a spacer sequence and a second direct repeat sequence. referring to the sequences in the 5′ to 3′ direction. In some embodiments, the first and second direct repeats of such an RNA guide are identical. In some embodiments, the first and second direct repeats of such an RNA guide are different.

In some embodiments, the spacer sequence and the direct repeat sequence(s) of the RNA guide are present within the same RNA molecule. In some embodiments, the spacer and direct repeat sequences are linked directly to one another. In some embodiments, a short linker is present between the spacer and direct repeat sequences, e.g., an RNA linker of 1, 2, or 3 nucleotides in length. In some embodiments, the spacer sequence and the direct repeat sequence(s) of the RNA guide are present in separate molecules, which are joined to one another by base pairing interactions.

Additional information regarding exemplary direct repeat and spacer components of RNA guides is provided as follows.

Direct Repeat

In some embodiments, the RNA guide comprises a direct repeat sequence. In some embodiments, the direct repeat sequence of the RNA guide has a length of between 12-100, 13-75, 14-50, or 15-40 nucleotides (e.g., 15, 16, 17, 18, 19, 20, 21, 22, 23, 24, 25, 26, 27.28, 29, 30, 31, 32, 33, 34, 35, 36, 37, 38, 39, or 40 nucleotides).

In some embodiments, the direct repeat sequence is or comprises a sequence of Table 1 or a portion of a sequence of Table 1. The direct repeat sequence can comprise nucleotide 1 through nucleotide 36 of any one of SEQ ID NOs: 1, 2, 3, 4, 5, 6, 7, or 8. The direct repeat sequence can comprise nucleotide 2 through nucleotide 36 of any one of SEQ ID NOs: 1, 2, 3, 4, 5, 6, 7, or 8. The direct repeat sequence can comprise nucleotide 3 through nucleotide 36 of any one of SEQ ID NOs: 1, 2, 3, 4, 5, 6, 7, or 8 The direct repeat sequence can comprise nucleotide 4 through nucleotide 36 of any one of SEQ ID NOs: 1, 2, 3, 4, 5, 6, 7, or 8. The direct repeat sequence can comprise nucleotide 5 through nucleotide 36 of any one of SEQ ID NOs: 1, 2, 3, 4, 5, 6, 7, or 8. The direct repeat sequence can comprise nucleotide 6 through nucleotide 36 of any one of SEQ ID NOs: 1, 2, 3, 4, 5, 6, 7, or 8. The direct repeat sequence can comprise nucleotide 7 through nucleotide 36 of any one of SEQ ID NOs: 1, 2, 3, 4, 5, 6, 7, or 8. The direct repeat sequence can comprise nucleotide 8 through nucleotide 36 of any one of SEQ ID NOs: 1, 2, 3, 4, 5, 6, 7, or 8. The direct repeat sequence can comprise nucleotide 9 through nucleotide 36 of any one of SEQ ID NOs: 1, 2, 3, 4, 5, 6, 7, or H. The direct repeat sequence can comprise nucleotide 10 through nucleotide 36 of any one of SEQ ID NOs: 1, 2, 3, 4, 5, 6, 7, or 8. The direct repeat sequence can comprise nucleotide 11 through nucleotide 36 of any one of SEQ ID NOs: 1, 2, 3, 4, 5, 6, 7, or 8. The direct repeat sequence can comprise nucleotide 12 through nucleotide 36 of any one of SEQ ID NOs: 1, 2, 3, 4, 5, 6, 7, or 8. The direct repeat sequence can comprise nucleotide 13 through nucleotide 36 of any one of SEQ ID NOs: 1, 2, 3, 4, 5, 6, 7, or 8. The direct repeat sequence can comprise nucleotide 14 through nucleotide 36 of any one of SEQ ID NOs: 1, 2, 3, 4, 5, 6, 7, or 8. The direct repeat sequence can comprise nucleotide 1 through nucleotide 34 of SEQ ID NO: 9. The direct repeat sequence can comprise nucleotide 2 through nucleotide 34 of SEQ ID NO: 9. The direct repeat sequence can comprise nucleotide 3 through nucleotide 34 of SEQ ID NO: 9. The direct repeat sequence can comprise nucleotide 4 through nucleotide 34 of SEQ ID NO: 9. The direct repeat sequence can comprise nucleotide 5 through nucleotide 34 of SEQ ID NO: 9. The direct repeat sequence can comprise nucleotide 6 through nucleotide 34 of SEQ ID NO: 9. The direct repeat sequence can comprise nucleotide 7 through nucleotide 34 of SEQ ID NO: 9. The direct repeat sequence can comprise nucleotide 8 through nucleotide 34 of SEQ ID NO: 9. The direct repeat sequence can comprise nucleotide 9 through nucleotide 34 of SEQ ID NO: 9. The direct repeat sequence can comprise nucleotide 10 through nucleotide 34 of SEQ ID NO: 9. The direct repeat sequence can comprise nucleotide 11 through nucleotide 34 of SEQ ID NO: 9. The direct repeat sequence can comprise nucleotide 12 through nucleotide 34 of SEQ ID NO: 9. In some embodiments, the direct repeat sequence is set forth in SEQ ID NO: 10. In some embodiments, the direct repeat sequence comprises a portion of the sequence set forth in SEQ ID NO: 10.

In some embodiments, the direct repeat sequence has or comprises a sequence comprising at least 90% identity (e.g., at least 90%, 91%, 92%, 93%, 94%, 95%, 96%, 97%, 98% or 99% identity) to a sequence of Table 1 or a portion of a sequence of Table 1. The direct repeat sequence can have or comprise a sequence having at least 90% identity to a sequence comprising nucleotide 1 through nucleotide 36 of any one of SEQ ID NOs: 1, 2, 3, 4, 5, 6, 7, or 8. The direct repeat sequence can have or comprise a sequence having at least 90% identity to a sequence comprising 2 through nucleotide 36 of any one of SEQ ID NOs: 1, 2, 3, 4, 5, 6, 7, or 8. The direct repeat sequence can have or comprise a sequence having at least 90% identity to a sequence comprising 3 through nucleotide 36 of any one of SEQ ID NOs: 1, 2, 3, 4, 5, 6, 7, or 8. The direct repeat sequence can have or comprise a sequence having at least 90% identity to a sequence comprising 4 through nucleotide 36 of any one of SEQ ID NOs: 1, 2, 3, 4, 5, 6, 7, or 8. The direct repeat sequence van have or comprise a sequence having at least 90% identify to a sequence comprising 5 through nucleotide 36 of any one of SEQ ID NOs: 1, 2, 3, 4, 5, 6, 7, or 8. The direct repeat sequence can have or comprise a sequence having at least 90% identity to a sequence comprising 6 through nucleotide 36 of any one of SEQ ID NOs: 1, 2, 3, 4, 5, 6, 7, or 8. The direct repeat sequence can have or comprise a sequence having at least 90% identity to a sequence comprising 7 through nucleotide 36 of any one of SEQ ID NOs: 1, 2, 3, 4, 5, 6, 7, or 8. The direct repeat sequence can have or comprise a sequence having at least 90% identity to a sequence comprising 8 through nucleotide 36 of any one of SEQ ID NOs: 1, 2, 3, 4, 5, 6, 7, or 8. The direct repeat sequence can have or comprise a sequence having at least 90% identity to a sequence comprising 9 through nucleotide 36 of any one of SEQ ID NOs: 1, 2, 3, 4, 5, 6, 7, or 8. The direct repeat sequence can have or comprise a sequence having at least 90% identity to a sequence comprising 10 through nucleotide 36 of any one of SEQ ID NOs: 1, 2, 3, 4, 5, 6, 7, or 8. The direct repeat sequence can have or comprise a sequence having at least 90% identity to a sequence comprising 11 through nucleotide 36 of any one of SEQ ID NOs: 1, 2, 3, 4, 5, 6, 7, or 8. The direct repeat sequence can have or comprise a sequence having at least 90% identity to a sequence comprising 12 through nucleotide 36 of any one of SEQ ID NOs: 1, 2, 3, 4, 5, 6, 7, or 8. The direct repeat sequence can have or comprise a sequence having at least 90% identity to a sequence comprising 13 through nucleotide 36 of any one of SEQ ID NOs: 1, 2, 3, 4, 5, 6, 7, or 8. The direct repeat sequence can have or comprise a sequence having at least 90% identity to a sequence comprising 14 through nucleotide 36 of any one of SEQ ID NOs: 1, 2, 3, 4, 5, 6, 7, or 8. The direct repeat sequence can have or comprise a sequence having at least 90% identity to a sequence comprising 1 through nucleotide 34 of SEQ ID NO: 9. The direct repeat sequence can have or comprise a sequence having at least 90% identity to a sequence comprising 2 through nucleotide 34 of SEQ ID NO: 9. The direct repeat sequence can have or comprise a sequence having at least 90% identity to a sequence comprising 3 through nucleotide 34 of SEQ ID NO: 9. The direct repeat sequence can have or comprise a sequence having at least 90% identity to a sequence comprising 4 through nucleotide 34 of SEQ ID NO: 9. The direct repeat sequence can have or comprise a sequence having at least 90% identity to a sequence comprising 5 through nucleotide 34 of SEQ ID NO: 9. The direct repeat sequence can have or comprise a sequence having at least 90% identity to a sequence comprising 6 through nucleotide 34 of SEQ ID NO: 9. The direct repeat sequence can have or comprise a sequence having at least 90% identity to a sequence comprising 7 through nucleotide 34 of SEQ ID NO: 9. The direct repeat sequence can have or comprise a sequence having at least 90% identity to a sequence comprising 8 through nucleotide 34 of SEQ ID NO: 9. The direct repeat sequence can have or comprise a sequence having at least 90% identity to a sequence comprising 9 through nucleotide 34 of SEQ ID NO: 9. The direct repeat sequence can have or comprise a sequence having at least 90% identity to a sequence comprising 10 through nucleotide 34 of SEQ ID NO: 9. The direct repeat sequence can have or comprise a sequence having at least 90% identity to a sequence comprising 11 through nucleotide 34 of SEQ ID NO: 9. The direct repeat sequence can have or comprise a sequence having at least 90% identity to a sequence comprising 12 through nucleotide 34 of SEQ ID NO: 9. In some embodiments, the direct repeat sequence has at least 90% identity (e.g., at least 90%, 91%, 92%, 93%, 94%, 95%, 96%, 97%, 98% or 99% identity) to SEQ ID NO: 10 In some embodiments, the direct repeat sequence has at least 90% identity to a portion of the sequence set forth in SEQ ID NO: 10.

In some embodiments, compositions comprising a Cas12i2 polypeptide and an RNA guide comprising the direct repeat of SEQ ID NO: 10 and a spacer length of 20 nucleotides are capable of introducing indels into a TRAC target sequence. See, e.g., Example 1, where indels were measured at seventeen TRAC target sequences following transient transfection of an RNA guide and Cas12i2 polypeptide of SEQ ID NO: 348, and Example 2, wherein indels were measured at four TRAC target sequences following delivery of an RNA guide and Cas12i2 polypeptide of SEQ ID NO: 349 by RNP.

In some embodiments, the direct repeat sequence is or comprises a sequence that is at least 90% identical to the reverse complement of any one of SEQ ID NOs: 1-10. In some embodiments, the direct repeat sequence is or comprises the reverse complement of any one of SEQ ID NOs: 1-10.

TABLE 1
Cas12i2 direct repeat sequences

| Sequence | |
|---|---|
| identifier | Direct Repeat Sequence |
| SEQ ID NO: | GUUGCAAAACCCAAGAAAUCCGUCUUUCAUUGACGG |
| 1 | |
| SEQ ID NO: | AAUAGCGGCCCUAAGAAAUCCGUCUUUCAUUGACGG |
| 2 | |
| SEQ ID NO: | AUUGGAACUGGCGAGAAAUCCGUCUUUCAUUGACGG |
| 3 | |
| SEQ ID NO: | CCAGCAACACCUAAGAAAUCCGUCUUUCAUUGACGG |
| 4 | |
| SEQ ID NO: | CGGCGCUCGAAUAGGAAAUCCGUCUUUCAUUGACGG |
| 5 | |
| SEQ ID NO: | GUGGCAACACCUAAGAAAUCCGUCUUUCAUUGACGG |
| 6 | |
| SEQ ID NO: | GUUGCAACACCUAAGAAAUCCGUCUUUCAUUGACGG |
| 7 | |
| SEQ ID NO: | GUUGCAAUGCCUAAGAAAUCCGUCUUUCAUUGACGG |
| 8 | |
| SEQ ID NO: | GCAACACCUAAGAAAUCCGUCUUUCAUUGACGGG |
| 9 | |
| SEQ ID NO: | AGAAAUCCGUUUUCAUUGACGG |
| 10 | |

In some embodiments, the direct repeat sequence is a sequence of Table 2 or a portion of a sequence of Table 2. The direct repeat sequence can comprise nucleotide 1 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 2 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 3 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 4 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 5 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 6 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 7 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 8 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 9 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 10 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 11 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 12 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 13 through nucleotide 36 of any one of SEQ ID NOs: 359, 360, 361, 362, 363, 364, 365, 366, 367, 368, 369, 370, 371, 372, 373, 374, 375, or 376. The direct repeat sequence can comprise nucleotide 14 through nucleotide 36 of any one of SEQ ID NOs: 359, 360, 361, 362, 363, 364, 365, 366, 367, 368, 369, 370, 371, 372, 373, 374, 375, or 376.

In some embodiments, the direct repeat sequence has at least 95% identity (e.g., at least 95%, 96%, 97%, 98% or 99% identity) to a sequence of Table 2 or a portion of a sequence of Table 2. The direct repeat sequence can have at least 95% identity to a sequence comprising nucleotide 1 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 2 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 3 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 4 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 5 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 6 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 7 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 8 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 9 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 10 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 11 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 12 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 95% identity to a sequence comprising 13 through nucleotide 36 of any one of SEQ ID NOs: 359, 360, 361, 362, 363, 364, 365, 366, 367, 368, 369, 370, 371, 372, 373, 374, 375, or 376.

In some embodiments, the direct repeat sequence has at least 90% identity (e.g., at least 90%, 91%, 92%, 93%, 94%, 95%, 96%, 97%, 98% or 99% identity) to a sequence of Table 2 or a portion of a sequence of Table 2. The direct repeat sequence can have at least 90% identity to a sequence comprising nucleotide 1 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 90% identity to a sequence comprising 2 through nucleotide 36 of any one of SEQ ID NOs: 359, 360, 361, 362, 363, 364, 365, 366. 367, 368, 369, 370, 371, 372, 373, 374, 375, or 376. The direct repeat sequence can have at least 90% identity to a sequence comprising 3 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 90% identity to a sequence comprising 4 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 90f identity to a sequence comprising 5 through nucleotide 36 of any one of SEQ ID NOs: 359, 360, 361, 362, 363, 364, 365, 366, 367, 368, 369, 370, 371, 372, 373, 374, 375. or 376. The direct repeat sequence can have at least 90% identity to a sequence comprising 6 through nucleotide 36 of any one of SEQ ID NOs: 359, 360, 361, 362, 363, 364, 365, 366, 367.368, 369, 370, 371, 372, 373, 374, 375, or 376. The direct repeat sequence can have at least 90% identity to a sequence comprising 7 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 90% identity to a sequence comprising 8 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 90% identity to a sequence comprising 9 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 90% identity to a sequence comprising 10 through nucleotide 36 of any one of SEQ ID NOs: 359, 360, 361, 362, 363, 364, 365, 366, 367, 368, 369, 370, 371, 372, 373, 374, 375. or 376. The direct repeat sequence can have at least 90% identity to a sequence comprising 11 through nucleotide 36 of any one of SEQ ID NOs: 359, 360, 361, 362, 363, 364, 365, 366.367, 368, 369, 370, 371, 372, 373, 374, 375, or 376. The direct repeat sequence can have at least 90% identity to a sequence comprising 12 through nucleotide 36 of any one of SEQ ID NOs: 359, 360, 361, 362, 363, 364, 365, 366, 367, 368, 369, 370, 371, 372, 373, 374, 375, or 376. The direct repeat sequence can have at least 90% identity to a sequence comprising 13 through nucleotide 36 of any one of SEQ ID NOs: 359, 360, 361, 362, 363, 364, 365, 366, 367, 368, 369, 370, 371, 372, 373, 374, 375, or 376.

In some embodiments, the direct repeat sequence is at least 90% identical to the reverse complement of any one of SEQ ID NOs: 359, 360, 361, 362, 363, 364, 365, 366, 367, 368, 369, 370, 371, 372, 373, 374, 375, or 376. In some embodiments, the direct repeat sequence is at least 95% identical to the reverse complement of any one of SEQ ID NOs: 359, 360, 361, 362, 363, 364, 365, 366, 367, 368, 369, 370, 371, 372, 373, 374, 375, or 376. In some embodiments, the direct repeat sequence is the reverse complement of any one of SEQ ID NOs: 359, 360, 361, 362, 363, 364, 365, 366, 367, 368, 369, 370, 371, 372, 373, 374, 375, or 376.

In some embodiments, the direct repeat sequence is at least 90% identical to SEQ ID NO: 377 or a portion of SEQ ID NO: 377. In some embodiments, the direct repeat sequence is at least 95% identical to SEQ ID NO: 377 or a portion of SEQ ID NO: 377. In some embodiments, the direct repeat sequence is 100% identical to SEQ ID NO: 377 or a portion of SEQ ID NO: 377.

TABLE 2
Cas12i4 direct repeat sequences.

| Sequence | |
|---|---|
| identifier | Direct Repeat Sequence |
| SEQ ID NO: | UCUCAACGAUAGUCAGACAUGUGUCCUCAGUGACAC |
| 359 | |
| SEQ ID NO: | UUUUAACAACACUCAGGCAUGUGUCCACAGUGACAC |
| 360 | |
| SEQ ID NO: | UUGAACGGAUACUCAGACAUGUGUUUCCAGUGACAC |
| 361 | |
| SEQ ID NO: | UGCCCUCAAUAGUCAGAUGUGUGUCCACAGUGACAC |
| 362 | |
| SEQ ID NO: | UCUCAAUGAUACUUAGAUACGUGUCCUCAGUGACAC |
| 363 | |
| SEQ ID NO: | UCUCAAUGAUACUCAGACAUGUGUCCCCAGUGACAC |
| 364 | |
| SEQ ID NO: | UCUCAAUGAUACUAAGACAUGUGUCCUCAGUGACAC |
| 365 | |
| SEQ ID NO: | UCUCAACUAUACUCAGACAUGUGUCCUCAGUGACAC |
| 366 | |
| SEQ ID NO: | UCUCAACGAUACUCAGACAUGUGUCCUCAGUGACAC |
| 367 | |
| SEQ ID NO: | UCUCAACGAUACUAAGAUAUGUGUCCUCAGCGACAC |
| 368 | |
| SEQ ID NO: | UCUCAACGAUACUAAGAUAUGUGUCCCCAGUGACAC |
| 369 | |
| SEQ ID NO: | UCUCAACGAUACUAAGAUAUGUGUCCACAGUGACAC |
| 370 | |
| SEQ ID NO: | UCUCAACAAUACUCAGACAUGUGUCCCCAGUGACAC |
| 371 | |
| SEQ ID NO: | UCUCAACAACACUAAGGCAUGUGUCCCCAGUGACCC |
| 372 | |
| SEQ ID NO: | UCUCAAAGAUACUCAGACACGUGUCCCCAGUGACAC |
| 373 | |
| SEQ ID NO: | UCUCAAAAAUACUCAGACAUGUGUCCUCAGUGACAC |
| 374 | |
| SEQ ID NO: | GCGAAACAACAGUCAGACAUGUGUCCCCAGUGACAC |
| 375 | |
| SEQ ID NO: | CCUCAACGAUAUUAAGACAUGUGUCCGCAGUGACAC |
| 376 | |
| SEQ ID NO: | AGACAUGUGUCCUCAGUGACAC |
| 377 | |

In some embodiments, the direct repeat sequence is a sequence of Table 3 or a portion of a sequence of Table 3. In some embodiments, the direct repeat sequence has at least 95% identity (e.g., at least 95%, 96%, 97%, 98% or 99% identity) to a sequence of Table 3 or a portion of a sequence of Table 3. In some embodiments, the direct repeat sequence has at least 90% identity (e.g., at least 90%, 91%, 92%, 93%, 94%, 95%, 96%, 97%, 98% or 99% identity) to a sequence of Table 3 or a portion of a sequence of Table 3. In some embodiments, the direct repeat sequence is at least 90% identical to the reverse complement of any one of SEQ ID NOs: 378-380. In some embodiments, the direct repeat sequence is at least 95% identical to the reverse complement of any one of SEQ ID) NOs: 378-380. In some embodiments, the direct repeat sequence is the reverse complement of any one of SEQ ID NOs: 378-380.

TABLE 3
Cas12i1 direct repeat sequences.

| Sequence | |
|---|---|
| identifier | Direct Repeat Sequence |
| SEQ ID NO: 378 | GUUGGAAUGACUAAUUUUUGUGCC |
| | CACCGUUGGCAC |
| SEQ ID NO: 379 | AAUUUUUGUGCCCAUCGUUGGCAC |
| SEQ ID NO: 380 | AUUUUUGUGCCCAUCGUUGGCAC |

In some embodiments, the direct repeat sequence is a sequence of Table 4 or a portion of a sequence of Table 4. In some embodiments, the direct repeat sequence has at least 95% identity (e.g., at least 95%, 96%, 97%, 98% or 99% identity) to a sequence of Table 4 or a portion of a sequence of Table 4. In some embodiments, the direct repeat sequence has at least 90% identity (e.g., at least 90%, 91%, 92%, 93%, 94%, S6%, 96%, 97%, 98% or 99% identity) to a sequence of Table 4 or a portion of a sequence of Table 4. In some embodiments, the direct repeat sequence is at least 90% identical to the reverse complement of any one of SEQ ID NOs: 381-383. In some embodiments, the direct repeat sequence is at least 95% identical to the reverse complement of any one of SEQ ID NOs: 381-383. In some embodiments, the direct repeat sequence is the reverse complement of any one of SEQ ID NOs: 381-383.

TABLE 4
Cas12i3 direct repeat sequences.

| Sequence | |
|---|---|
| identifier | Direct Repeat Sequence |
| SEQ ID NO: 381 | CUAGCAAUGACCUAAUAGUGUG |
| | UCCUUAGUUGACAU |
| SEQ ID NO: 382 | CCUACAAUACCUAAGAAAUCCG |
| | UCCUAAGUUGACGG |
| SEQ ID NO: 383 | AUAGUGUGUCCUUAGUUGACAU |

In some embodiments, a direct repeat sequence described herein comprises a uracil (U). In some embodiments, a direct repeat sequence described herein comprises a thymine (T). In some embodiments, a direct repeat sequence according to Tables 1-4 comprises a sequence comprising a thymine in one or more places indicated as uracil in Tables 1-4.

Spacer In some embodiments, the RNA guide comprises a DNA targeting or spacer sequence. In some embodiments, the spacer sequence of the RNA guide has a length of between 12-100, 13-75, 14-50, or 15-nucleotides (e.g., 15, 16, 17, 18, 19, 20, 21, 22, 23, 24, 25, 26, 27, 28, 29, or 30 nucleotides) and is complementary a specific target sequence. In some embodiments, the spacer sequence is designed to be complementary to a specific DNA strand, e.g., of a genomic locus.

In some embodiments, the RNA guide spacer sequence is substantially identical to a complementary strand of a target sequence. In some embodiments, the RNA guide comprises a sequence having at least about 60%, at least about 65%, at least about 70%, at least about 75%, at least about 80%. at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., target sequence. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide comprises a spacer sequence that has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and at least 80%, at least 90%, at least 95%, at least 96%, at least 97%, at least 98%, at least 99% complementary to a target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence. In some embodiments, the RNA guide comprises a sequence, e.g., RNA sequence, that is a length of up to 50 and at least 80%, at least 90%, at least 95%, at least 96%, at least 97%, at least 98%. at least 99% complementary to a target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In some embodiments, the spacer sequence is or comprises a sequence of Table 5 or a portion of a sequence of Table 5. The target sequences listed in Table 5 and Table 6 are on the non-target strand of the TRAC sequence. It should be understood that an indication of SEQ ID NOs: 174-336 should be considered as equivalent to a listing of SEQ ID NOs: 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, and 336.

The spacer sequence can comprise nucleotide 1 through nucleotide 16 of any one of SEQ ID NOs: 174-336. The spacer sequence can comprise nucleotide 1 through nucleotide 17 of any one of SEQ ID NOs: 174-336. The spacer sequence can comprise nucleotide 1 through nucleotide 18 of any one of SEQ ID NOs: 174-336. The spacer sequence can comprise nucleotide 1 through nucleotide 19 of any one of SEQ ID NOs: 174-336. The spacer sequence can comprise nucleotide 1 through nucleotide 20 of any one of SEQ ID NOs: 174-336. The spacer sequence can comprise nucleotide 1 through nucleotide 21 of any one of SEQ ID NOs: 174-336. The spacer sequence can comprise nucleotide 1 through nucleotide 22 of any one of SEQ ID NOs: 174-336. The spacer sequence can comprise nucleotide 1 through nucleotide 23 of any one of SEQ ID NOs: 174-336. The spacer sequence can comprise nucleotide 1 through nucleotide 24 of any one of SEQ ID NOs: 174-336. The spacer sequence can comprise nucleotide 1 through nucleotide 25 of any one of SEQ ID NOs: 174-275 and 277-336. The spacer sequence can comprise nucleotide 1 through nucleotide 26 of any one of SEQ ID NOs: 174-275 and 277-336. The spacer sequence can comprise nucleotide 1 through nucleotide 27 of any one of SEQ ID NOs: 174-275 and 277-336. The spacer sequence can comprise nucleotide 1 through nucleotide 28 of any one of SEQ ID NOs: 174-275 and 277-336. The spacer sequence can comprise nucleotide 1 though nucleotide 29 of any one of SEQ ID NOs: 174-275 and 277-336. The spacer sequence can comprise nucleotide 1 through nucleotide 30 of any one of SEQ ID NOs: 174-275 and 277-336.

In some embodiments, the spacer sequence has at least 90% identity (e.g., at least 90%, 91%, 92%, 93%, 94%, 95%, 96%, 97%, 98% or 99% identity) to a sequence of Table 5 or a portion of a sequence of Table 5. The spacer sequence can have or comprise a sequence having at least 90% identity to a sequence comprising nucleotide 1 through nucleotide 16 of any one of SEQ ID NOs: 174-336. The spacer sequence can have or comprise a sequence having at least 90% identity to a sequence comprising nucleotide 1 through nucleotide 17 of any one of SEQ ID NOs: 174-336. The spacer sequence can have or comprise a sequence having at least 90% identity to a sequence comprising nucleotide 1 through nucleotide 18 of any one of SEQ ID NOs: 174-336. The spacer sequence can have or comprise a sequence having at least 90% identity to a sequence comprising nucleotide 1 through nucleotide 19 of any one of SEQ ID NOs: 174-336. The spacer sequence can have or comprise a sequence having at least 90% identity to a sequence comprising nucleotide 1 through nucleotide 20 of any one of SEQ ID NOs: 174-336. The spacer sequence can have or comprise a sequence having at least 90% identity to a sequence comprising nucleotide 1 through nucleotide 21 of any one of SEQ ID NOs: 174-336. The spacer sequence can have or comprise a sequence having at least 90% identity to a sequence comprising nucleotide 1 through nucleotide 22 of any one of SEQ ID NOs: 174-336. The spacer sequence can have or comprise a sequence having at least 90% identity to a sequence comprising nucleotide 1 through nucleotide 23 of any one of SEQ ID NOs: 174-336. The spacer sequence can have or comprise a sequence having at least 90% identity to a sequence comprising nucleotide 1 through nucleotide 24 of any one of SEQ ID NOs: 174-336. The spacer sequence can have or comprise a sequence having at least 90% identity to a sequence comprising nucleotide 1 through nucleotide 25 of any one of SEQ ID NOs: 174-275 and 277-336. The spacer sequence can have or comprise a sequence having at least 90% identity to a sequence comprising nucleotide 1 though nucleotide 26 of any one of SEQ ID NOs: 174-275 and 277-336. The spacer sequence can have or comprise a sequence having at least 90% identity to a sequence comprising nucleotide 1 through nucleotide 27 of any one of SEQ ID NOs: 174-275 and 277-336. The spacer sequence can have or comprise a sequence having at least 90% identity to a sequence comprising nucleotide 1 through nucleotide 28 of any one of SEQ ID NOs: 174-275 and 277-336. The spacer sequence can have or comprise a sequence having at least 90% identity to a sequence comprising nucleotide 1 through nucleotide 29 of any one of SEQ ID NOs: 174-275 and 277-336. The spacer sequence can have or comprise a sequence having at least 90% identity to a sequence comprising nucleotide 1 through nucleotide 30 of any one of SEQ ID NOs: 174-275 and 277-336.

TABLE 5
TRAC target and spacer sequences

| TRAC | | | | |
|---|---|---|---|---|
| exon | strand | PAM | target sequence | spacer sequence |
| TRAC_ | − | CTTA | CTTTGTGACACATTTGTTTGAGAAT | CUUUGUGACACAUUUGUUUGAGAAUC |
| exon_1 | | | CAAAA (SEQ ID NO: 11) | AAAA (SEQ ID NO: 174) |
| TRAC_ | + | ATTC | ACCGATTTTGATTCTCAAACAAATG | ACCGAUUUUGAUUCUCAAACAAAUGU |
| exon_1 | | | TGTCA (SEQ ID NO: 12) | CUCA (SEQ ID NO: 175) |
| TRAC_ | + | ATTT | TGATTCTCAAACAAATGTGTCACAA | UGAUUCUCAAACAAAUGUGUCACAAA |
| exon_1 | | | AGTAA (SEQ ID NO: 13) | GUAA (SEQ ID NO: 176) |
| TRAC_ | + | TTTT | GATTCTCAAACAAATGTGTCACAAA | GAUUCUCAAACAAAUGUGUCACAAAG |
| exon_1 | | | GTAAG (SEQ ID NO: 14) | DAAG (SEQ ID NO: 177) |
| TRAC_ | + | TTTG | ATTCTCAAACAAATGTGTCACAAAG | AUUCUCAAACAAAUGUGUCACAAAGU |
| exon_1 | | | TAAGG (SEQ ID NO: 15) | AAGG (SEQ ID NO: 178) |
| TRAC_ | + | ATTC | TCAAACAAATGTGTCACAAAGTAAG | UCAAACAAAUGUGUCACAAAGUAAGG |
| exon_1 | | | GATTC (SEQ ID NO: 16) | AUUC (SEQ ID NO: 179) |
| TRAC_ | + | ATTC | TGATGTGTATATCACAGACAAAACT | UGAUGUGUAUAUCACAGACAAAACUG |
| exon_1 | | | GTGCT (SEQ ID NO: 17) | UGCU (SEQ ID NO: 180) |
| TRAC_ | + | CTTC | AAGAGCAACAGTGCTGTGGCCTGGA | AAGAGCAACAGUGCUGUGGCCUGGAG |
| exon_1 | | | GCAAC (SEQ ID NO: 18) | CAAC (SEQ ID NO: 181) |
| TRAC_ | + | CTTT | GCATGTGCAAACGCCTTCAACAACA | GCAUGUGCAAACGCCUUCAACAACAG |
| exon_1 | | | GCATT (SEQ ID NO: 19) | CAUU (SEQ ID NO: 182) |
| TRAC_ | + | TTTG | CATGTGCAAACGCCTTCAACAACAG | CAUGUGCAAACGCCUUCAACAACAGC |
| exon_1 | | | CATTA (SEQ ID NO: 20) | AUUA (SEQ ID NO: 183) |
| TRAC_ | + | CTTC | AACAACAGCATTATTCCAGAAGACA | AACAACAGCAUUAUUCCAGAAGACAC |
| exon_1 | | | CCTTC (SEQ ID NO: 21) | CUUC (SEQ ID NO: 184) |
| TRAC_ | + | ATTA | TTCCAGAAGACACCTTCTTCCCCAG | UUCCAGAAGACACCUUCUUCCCCAGC |
| exon_1 | | | CCCAG (SEQ ID NO: 22) | CCAG (SEQ ID NO: 185) |
| TRAC_ | + | ATTC | CAGAAGACACCTTCTTCCCCAGCCC | CAGAAGACACCUUCUUCCCCAGCCCA |
| exon_1 | | | AGGTA (SEQ ID NO: 23) | GGUA (SEQ ID NO: 186) |
| TRAC_ | + | CTTC | TTCCCCAGCCCAGGTAAGGGCAGCT | UUCCCCAGCCCAGGUAAGGGCAGCUU |
| exon_1 | | | TTGGT (SEQ ID NO: 24) | UGGU (SEQ ID NO: 187) |
| TRAC_ | + | CTTC | CCCAGCCCAGGTAAGGGCAGCTTTG | CCCAGCCCAGGUAAGGGCAGCUUUGG |
| exon_1 | | | GTGCC (SEQ ID NO: 25) | UGCC (SEQ ID NO: 188) |
| TRAC_ | + | CTTT | GGTGCCTTCGCAGGCTGTTTCCTTG | GGUGCCUUCGCAGGCUGUUUCCUUGC |
| exon_1 | | | CTTCA (SEQ ID NO: 26) | UUCA (SEQ ID NO: 189) |
| TRAC_ | + | TTTG | GTGCCTTCGCAGGCTGTTTCCTTGC | GUGCCUUCGCAGGCUGUUUCCUUGCU |
| exon_1 | | | TTCAG (SEQ ID NO: 27) | UCAG (SEQ ID NO: 190) |
| TRAC_ | + | CTTC | GCAGGCTGTTTCCTTGCTTCAGGAA | GCAGGCUGUUUCCUUGCUUCAGGAAU |
| exon_1 | | | TGGCC (SEQ ID NO: 28) | GGCC (SEQ ID NO: 191) |
| TRAC_ | − | ATTC | CTGAAGCAAGGAAACAGCCTGCGAA | CUGAAGCAAGGAAACAGCCUGCGAAG |
| exon_1 | | | GGCAC (SEQ ID NO: 29) | GCAC (SEQ ID NO: 192) |
| TRAC_ | − | CTTA | CCTGGGCTGGGGAAGAAGGTGTCTT | CCUGGGCUGGGGAAGAAGGUGUCUUC |
| exon_1 | | | CTGGA (SEQ ID NO: 30) | UGGA (SEQ ID NO: 193) |
| TRAC_ | + | CTTG | TCCCACAGATATCCAGAACCCTGAC | UCCCACAGAUAUCCAGAACCCUGACC |
| exon_1 | | | CCTGC (SEQ ID NO: 31) | CUGC (SEQ ID NO: 194) |
| TRAC_ | − | CTTC | TGGAATAATGCTGTTGTTGAAGGCG | UGGAAUAAUGCUGUUGUUGAAGGCGU |
| exon_1 | | | TTTGC (SEQ ID NO: 32) | UUGC (SEQ ID NO: 195) |
| TRAC_ | − | GTTG | TTGAAGGCGTTTGCACATGCAAAGT | UUGAAGGCGUUUGCACAUGCAAAGUC |
| exon_1 | | | CAGAT (SEQ ID NO: 33) | AGAU (SEQ ID NO: 195) |
| TRAC_ | − | GTTT | GCACATGCAAAGTCAGATTTGTTGC | GCACAUGCAAAGUCAGAUUUGUUGCU |
| exon_1 | | | TCCAG (SEQ ID NO: 34) | CCAG (SEQ ID NO: 197) |
| TRAC_ | − | TTTG | CACATGCAAAGTCAGATTTGTTGCT | CACAUGCAAAGUCAGAUUUGUUGCUC |
| exon_1 | | | CCAGG (SEQ ID NO: 35) | CAGG (SEQ ID NO: 198) |
| TRAC_ | − | ATTT | GTTGCTCCAGGCCACAGCACTGTTG | GUUGCUCCAGGCCACAGCACUGUUGC |
| exon_1 | | | CTCTT (SEQ ID NO: 36) | UCUU (SEQ ID NO: 199) |
| TRAC_ | − | TTTG | TTGCTCCAGGCCACAGCACTGTTGC | UUGCUCCAGGCCACAGCACUGUUGCU |
| exon_1 | | | TCTTG (SEQ ID NO: 37) | CUUG (SEQ ID NO: 200) |
| TRAC_ | − | GTTG | CTCCAGGCCACAGCACTGTTGCTCT | CUCCAGGCCACAGCACUGUUGCUCUU |
| exon_1 | | | TGAAG (SEQ ID NO: 38) | GAAG (SEQ ID NO: 201) |
| TRAC_ | − | GTTG | CTCTTGAAGTCCATAGACCTCATGT | CUCUUGAAGUCCAUAGACCUCAUGUC |
| exon_3 | | | CTAGC (SEQ ID NO: 39) | UAGC (SEQ ID NO: 202) |
| TRAC_ | − | CTTG | AAGTCCATAGACCTCATGTCTAGCA | AAGUCCAUAGACCUCAUGUCUAGCAC |
| exon_1 | | | CAGTT (SEQ ID NO: 40) | AGUU (SEQ ID NO: 203) |
| TRAC_ | − | GTTT | TGTCTGTGATATACACATCAGAATC | UGUCUGUGAUAUACACAUCAGAAUCC |
| exon_1 | | | CTTAC (SEQ ID NO: 41) | QUAC (SEQ ID NO: 204) |
| TRAC_ | − | TTTT | GTCTGTGATATACACATCAGAATCC | CUCUGUGAUAUACACAUCAGAAUCCU |
| exon_1 | | | TTACT (SEQ ID NO: 42) | CACU (SEQ ID NO: 205) |
| TRAC_ | − | TTTG | TCTGTGATATACACATCAGAATCCT | UCUGUGAUAUACACAUCAGAAUCCUU |
| exon_1 | | | TACTT (SEQ ID NO: 43) | ACUU (SEQ ID NO: 206) |
| TRAC_ | − | CTTT | GTGACACATTTGTTTGAGAATCAAA | GUGACACAUUUGUUUGAGAAUCAAAA |
| exon_1 | | | ATCGG (SEQ ID NO: 44) | UCGG (SEQ ID NO: 207) |
| TRAC_ | − | GTTG | AAGGCGTTTGCACATGCAAAGTCAG | AAGGCGUUUGCACAUGCAAAGUCAGA |
| exon_1 | | | ATTTG (SEQ ID NO: 45) | UUUG (SEQ ID NO: 208) |
| TRAC_ | − | TTTG | TGACACATTTGTTTGAGAATCAAAA | UGACACAUUUGUUUGAGAAUCAAAAU |
| exon_1 | | | TCGGT (SEQ ID NO: 46) | CGGU (SEQ ID NO: 209) |
| TRAC_ | − | ATTT | GTTTGAGAATCAAAATCGGTGAATA | GUUUGAGAAUCAAAAUCGGUGAAUAG |
| exon_1 | | | GGCAG (SEQ ID NO: 47) | GCAG (SEQ ID NO: 210) |
| TRAC_ | − | TTTG | TTTGAGAATCAAAATCGGTGAATAG | UUUGAGAAUCAAAAUCGGUGAAUAGG |
| exon_1 | | | GCAGA (SEQ ID NO: 48) | CAGA (SEQ ID NO: 211) |
| TRAC_ | − | GTTT | GAGAATCAAAATCGGTGAATAGGCA | GAGAAUCAAAAUCGGUGAAUAGGCAG |
| exon_1 | | | GACAG (SEQ ID NO: 49) | ACAG (SEQ ID NO: 212) |
| TRAC_ | − | TTTG | AGAATCAAAATCGGTGAATAGGCAG | AGAAUCAAAAUCGGUGAAUAGGCAGA |
| exon_1 | | | ACAGA (SEQ ID NO: 50) | CAGA (SEQ ID NO: 213) |
| TRAC_ | − | CTTG | TCACTGGATTTAGAGTCTCTCAGCT | UCACUGGAUUUAGAGUCUCUCAGCUG |
| exon_1 | | | GGTAC (SEQ ID NO: 51) | GUAC (SEQ ID NO: 214) |
| TRAC_ | − | ATTT | AGAGTCTCTCAGCTGGTACACGGCA | AGAGUCUCUCAGCUGGUACACGGCAG |
| exon_1 | | | GGGTC (SEQ ID NO: 52) | GGUC (SEQ ID NO: 215) |
| TRAC_ | − | TTTA | GAGTCTCTCAGCTGGTACACGGCAG | GAGUCUCUCAGCUGGUACACGGCAGG |
| exon_1 | | | GGTCA (SEQ ID NO: 53) | CUCA (SEQ ID NO: 216) |
| TRAC_ | − | GTTC | TGGATATCTGTGGGACAAGAGGATC | UGGAUAUCUGUGGGACAAGAGGAUCA |
| exon_1 | | | AGGGT (SEQ ID NO: 54) | GGGU (SEQ ID NO: 217) |
| TRAC_ | − | GTTA | GGACATGATCTCATTTCCCTCTTTG | GGACAUGAUCUCAUUUCCCUCUUUGC |
| exon_1 | | | CCCCA (SEQ ID NO: 55) | CCCA (SEQ ID NO: 218) |
| TRAC_ | − | TTTC | AGCATCCAGAGCAGGCTGACTCTCC | AGCAUCCAGAGCAGGCUGACUCUCCC |
| exon_2 | | | CACCC (SEQ ID NO: 56) | ACCC (SEQ ID NO: 219) |
| TRAC_ | + | GTTT | TTCCTTTTAGAAAGTTCCTGTGATG | UUCCUUUUAGAAAGUUCCUGUGAUGU |
| exOn 2 | | | TCAAG (SEQ ID NO: 57) | CAAG (SEQ ID NO: 220) |
| TRAC_ | − | CTTT | CAGCATCCAGAGCAGGCTGACTCTC | CAGCAUCCAGAGCAGGCUGACUCUCC |
| exon_2 | | | CCACC (SEQ ID NO: 58) | CACC (SEQ ID NO: 221) |
| TRAC_ | − | TTTC | TAAAAGGAAAAACAGACATTCTTTC | UAAAAGGAAAAACAGACAUUCUUUCA |
| exon_2 | | | AGCAT (SEQ ID NO: 59) | GCAU (SEQ ID NO: 222) |
| TRAC_ | + | TTTT | TCCTTTTAGAAAGTTCCTGTGATGT | UCCUUUUAGAAAGUUCCUGUGAUGUC |
| exon_2 | | | CAAGC (SEQ ID NO: 60) | AAGC (SEQ ID NO: 223) |
| TRAC_ | + | TTTT | CCTTTTAGAAAGTTCCTGTGATGTC | CCUUUUAGAAAGUUCCUGUGAUGUCA |
| exon_2 | | | AAGCT (SEQ ID NO: 61) | AGCU (SEQ ID NO: 224) |
| TRAC_ | + | TTTC | CTTTTAGAAAGTTCCTGTGATGTCA | CUUUUAGAAAGUUCCUGUGAUGUCAA |
| exon_2 | | | AGCTG (SEQ ID NO: 62) | GCUG (SEQ ID NO: 225) |
| TRAC_ | + | CTTT | TAGAAAGTTCCTGTGATGTCAAGCT | UAGAAAGUUCCUGUGAUGUCAAGCUG |
| exon_2 | | | GGTCG (SEQ ID NO: 63) | GUCG (SEQ ID NO: 225) |
| TRAC_ | + | TTTT | AGAAAGTTCCTGTGATGTCAAGCTG | AGAAAGUUCCUGUGAUGUCAAGCUGG |
| exon_2 | | | GTCGA (SEQ ID NO: 64) | UCGA (SEQ ID NO: 227) |
| TRAC_ | + | TTTA | GAAAGTTCCTGTGATGTCAAGCTGG | GAAAGUUCCUGUGAUGUCAAGCUGGU |
| exon_2 | | | TCGAG (SEQ ID NO: 65) | CGAG (SEQ ID NO: 228) |
| TRAC_ | + | GTTC | CTGTGATGTCAAGCTGGTCGAGAAA | CUGUGAUGUCAAGCUGGUCGAGAAAA |
| exon_2 | | | AGCTT (SEQ ID NO: 66) | CCUU (SEQ ID NO: 229) |
| TRAC_ | + | CTTT | GAAACAGGTAAGACAGGGGTCTAGC | CAAACAGGUAAGACAGGGGUCUAGCC |
| exon_2 | | | CTGGG (SEQ ID NO: 67) | UGGG (SEQ ID NO: 230) |
| TRAC_ | + | TTTG | AAACAGGTAAGACAGGGGTCTAGCC | AAACAGGUAAGACAGGGGUCUAGCCU |
| exon_2 | | | TGGGT (SEQ ID NO: 68) | GGGU (SEQ ID NO: 231) |
| TRAC_ | + | GTTT | GCACAGGATTGCGGAAGTGATGAAC | GCACAGGAUUGCGGAAGUGAUGAACC |
| exon_2 | | | CCGCA (SEQ ID NO: 69) | CGCA (SEQ ID NO: 232) |
| TRAC_ | − | CTTC | CGCAATCCTGTGCAAACCCAGGCTA | CGCAAUCCUGUGCAAACCCAGGCUAG |
| exon_2 | | | GACCC (SEQ ID NO: 70) | ACCC (SEQ ID NO: 233) |
| TRAC_ | − | CTTA | CCTGTTTCAAAGCTTTTCTCGACCA | CCUGUUUCAAAGCUUUUCUCGACCAG |
| exon_2 | | | GCTTG (SEQ ID NO: 71) | CUUG (SEQ ID NO: 234) |
| TRAC_ | − | GTTT | CAAAGCTTTTCTCGACCAGCTTGAC | CAAAGCUUUUCUCGACCAGCUUGACA |
| exon_2 | | | ATCAC (SEQ ID NO: 72) | UCAC (SEQ ID NO: 235) |
| TRAC_ | − | TTTC | AAAGCTTTTCTCGACCAGCTTGACA | AAAGCUUUUCUCGACCAGCUUGACAU |
| exon_2 | | | TCACA (SEQ ID NO: 73) | CACA (SEQ ID NO: 236) |
| TRAC_ | − | CTTT | TCTCGACCAGCTTGACATCACAGGA | UCUCGACCAGCUUGACAUCACAGGAA |
| exon_2 | | | ACTTT (SEQ ID NO: 74) | CUUU (SEQ ID NO: 237) |
| TRAC_ | − | TTTT | CTCGACCAGCTTGACATCACAGGAA | CUCGACCAGCUUGACAUCACAGGAAC |
| exon_2 | | | CTTTC (SEQ ID NO: 75) | UUUC (SEQ ID NO: 238) |
| TRAC_ | − | TTTC | TCGACCAGCTTGACATCACAGGAAC | UCGACCAGCUUGACAUCACAGGAACU |
| exon_2 | | | TTCT (SEQ ID NO: 76) | UUCU (SEQ ID NO: 239) |
| TRAC_ | − | CTTG | ACATCACAGGAACTTTCTAAAAGGA | ACAUCACAGGAACUUUCUAAAAGGAA |
| exon_2 | | | AAAAC (SEQ ID NO: 77) | AAAC (SEQ ID NO: 240) |
| TRAC_ | − | CTTT | CTAAAAGGAAAAACAGACATTCTTT | CUAAAAGGAAAAACAGACAUUCUUUC |
| exon_2 | | | CAGCA (SEQ ID NO: 78) | AGCA (SEQ ID NO: 241) |
| TRAC_ | − | ATTC | TTTCAGCATCCAGAGCAGGCTGACT | UUUCAGCAUCCAGAGCAGGCUGACUC |
| exon_2 | | | CTCCC (SEQ ID NO: 79) | UCCC (SEQ ID NO: 242) |
| TRAC_ | + | TTTG | CACAGGATTGCGGAAGTGATGAACC | CACAGGAUUGCGGAAGUGAUGAACCC |
| exon_2 | | | CGCAA (SEQ ID NO: 80) | GCAA (SEQ ID NO: 243) |
| TRAC_ | + | CTTG | GTTTTACAGATACGAACCTAAACTT | GUUUUACAGAUACGAACCUAAACUUU |
| exon_3 | | | TCAAA (SEQ ID NO: 81) | CAAA (SEQ ID NO: 244) |
| TRAC_ | − | GTTC | GTATCTGTAAAACCAAGAGGCCACA | GUAUCUGUAAAACCAAGAGGCCACAG |
| exon_3 | | | GCGGT (SEQ ID NO: 82) | CGGU (SEQ ID NO: 245) |
| TRAC_ | − | GTTA | TGGGCTTGCATGTCCCAGGCCTGCA | UGGGCUUGCAUGUCCCAGGCCUGCAU |
| exon_3 | | | TGGCC (SEQ ID NO: 83) | GGCC (SEQ ID NO: 246) |
| TRAC_ | + | GTTT | TACAGATACGAACCTAAACTTTCAA | DACAGAUACGAACCUAAACUUUCAAA |
| exon_3 | | | AACCT (SEQ ID NO: 84) | ACCU (SEQ ID NO: 247) |
| TRAC_ | + | TTTT | ACAGATACGAACCTAAACTTTCAAA | ACAGAUACGAACCUAAACUUUCAAAA |
| exon_3 | | | ACCTG (SEQ ID NO: 85) | CCUG (SEQ ID NO: 248) |
| TRAC_ | + | TTTA | CAGATACGAACCTAAACTTTCAAAA | CAGAUACGAACCUAAACUUUCAAAAC |
| exon_3 | | | CCTGT (SEQ ID NO: 86) | CUGU (SEQ ID NO: 249) |
| TRAC_ | + | TTTC | AAAACCTGTCAGTGATTGGGTTCCG | AAAACCUGUCAGUGAUUGGGUUCCGA |
| exon_3 | | | AATCC (SEQ ID NO: 87) | AUCC (SEQ ID NO: 250) |
| TRAC_ | + | ATTG | GGTTCCGAATCCTCCTCCTGAAAGT | GGUUCCGAAUCCUCCUCCUGAAAGUG |
| exon_3 | | | GGCCG (SEQ ID NO: 88) | GCCG (SEQ ID NO: 251) |
| TRAC_ | + | GTTC | CGAATCCTCCTCCTGAAAGTGGCCG | CGAAUCCUCCUCCUGAAAGUGGCCGG |
| exon_3 | | | GGTTT (SEQ ID NO: 89) | GUUU (SEQ ID NO: 252) |
| TRAC_ | + | GTTT | AATCTGCTCATGACGCTGCGGCTGT | AAUCUGCUCAUGACGCUGCGGCUGUG |
| exon_3 | | | GGTCC (SEQ ID NO: 90) | GUCC (SEQ ID NO: 253) |
| TRAC_ | + | TTTA | ATCTGCTCATGACGCTGCGGCTGTG | AUCUGCUCAUGACGCUGCGGCUGUGG |
| exon_3 | | | GTCCA (SEQ ID NO: 91) | UCCA (SEQ ID NO: 254) |
| TRAC_ | + | CTTG | AAGCTGGGAGTGGGGTTTAGGGACG | AAGCUGGGAGUGGGGUUUAGGGACGC |
| exon_3 | | | CGGGT (SEQ ID NO: 92) | GGGU (SEQ ID NO: 255) |
| TRAC_ | + | CTTT | CAAAACCTGTCAGTGATTGGGTTCC | CAAAACCUGUCAGUGAUUGGGUUCCG |
| exon_3 | | | GAATC (SEQ ID NO: 93) | AAUC (SEQ ID NO: 256) |
| TRAC_ | + | ATTA | AACCCGGCCACTTTCAGGAGGAGGA | AACCCGGCCACUUUCAGGAGGAGGAU |
| exon_3 | | | TTCGG (SEQ ID NO: 94) | UCGG (SEQ ID NO: 257) |
| TRAC_ | − | CTTT | CAGGAGGAGGATTOGGAACCCAATC | CAGGAGGAGGAUUCGGAACCCAAUCA |
| exon_3 | | | ACTGA (SEQ ID NO: 95) | CUGA (SEQ ID NO: 258) |
| TRAC_ | − | TTTC | AGGAGGAGGATTCGGAACCCAATCA | AGGAGGAGGAUUCGGAACCCAAUCAC |
| exon_3 | | | CTGAC (SEQ ID NO: 96) | UGAC (SEQ ID NO: 259) |
| TRAC_ | − | ATTC | GGAACCCAATCACTGACAGGTTTTG | @GAACCCAAUCACUGACAGGUUUUGA |
| exon_3 | | | AAAGT (SEQ ID NO: 97) | AAGU (SEQ ID NO: 260) |
| TRAC_ | − | GTTT | TGAAAGTTTAGGTTCGTATCTGTAA | UGAAAGUUUAGGUUCGUAUCUGUAAA |
| exon_3 | | | AACCA (SEQ ID NO: 98) | ACCA (SEQ ID NO: 261) |
| TRAC_ | − | TTTT | GAAAGTTTAGGTTCGTATCTGTAAA | GAAAGUUUAGGUUCGUAUCUGUAAAA |
| exon_3 | | | ACCAA (SEQ ID NO: 99) | CCAA (SEQ ID NO: 262) |
| TRAC_ | − | TTTG | AAAGTTTAGGTTCGTATCTGTAAAA | AAAGUUUAGGUUCGUAUCUGUAAAAC |
| exon_3 | | | CCAAG (SEQ ID NO: 100) | CAAG (SEQ ID NO: 263) |
| TRAC_ | − | GTTT | AGGTTCGTATCTGTAAAACCAAGAG | AGGUUCGUAUCUGUAAAACCAAGAGG |
| exon_3 | | | GCCAC (SEQ ID NO: 101) | CCAC (SEQ ID NO: 264) |
| TRAC_ | − | CTTC | AAGGCCCCTCACCTCAGCTGGACCA | AAGGCCCCUCACCUCAGCUGGACCAC |
| exon_3 | | | CAGCC (SEQ ID NO: 102) | AGCC (SEQ ID NO: 265) |
| TRAC_ | − | TTTA | GGTTCGTATCTGTAAAACCAAGAGG | GGUUCGUAUCUGUAAAACCAAGAGGC |
| exon_3 | | | CCACA (SEQ ID NO: 103) | CACA (SEQ ID NO: 266) |
| TRAC_ | + | CTTC | TCAAGACGTGGGGGGAAATTATCTC | UCAAGACGUGGGGGGAAAUUAUCUCA |
| exon_4 | | | ATTAT (SEQ ID NO: 104) | UUAU (SEQ ID NO: 267) |
| TRAC_ | − | ATTT | CTTTATGAACACTATTAAAAAAAAA | CUUUAUGAACACUAUUAAAAAAAAAU |
| exon_4 | | | TAAAC (SEQ ID NO: 105) | AAAC (SEQ ID NO: 268) |
| TRAC_ | − | CTTG | AGAAGAAGAATACTATGTATTTCTT | AGAAGAAGAAUACUAUGUAUUUCUUU |
| exon_4 | | | TATGA (SEQ ID NO: 106) | AUGA (SEQ ID NO: 269) |
| TRAC_ | − | TTTC | CCCCCACGTCTTGAGAAGAAGAATA | CCCCCACGUCUUGAGAAGAAGAAUAC |
| exon_4 | | | CTATG (SEQ ID NO: 107) | UAUG (SEç ID NO: 270) |
| TRAC_ | − | ATTT | CCCCCCACGTCTTGAGAAGAAGAAT | CCCCCCACGUCUUGAGAAGAAGAAUA |
| exon_4 | | | ACTAT (SEQ ID NO: 108 | CUAU (SEQ ID NO: 271) |
| TRAC_ | − | TITA | ATGAAGGCATCGGCAGCAGGACATA | AUGAAGGCAUCGGCAGCAGGACAUAC |
| exon_4 | | | CAACA (SEQ ID NO: 109) | AACA (SEQ ID NO: 272) |
| TRAC_ | + | GTTG | TATGTCCTGCTGCCGATGCCTTCAT | UAUGUCCUGCUGCCGAUGCCUUCAUU |
| exon_4 | | | TAAAA (SEQ ID NO: 110) | AAAA (SEQ ID NO: 273) |
| TRAC_ | − | ATTT | TAATGAAGGCATCGGCAGCAGGACA | UAAUGAAGGCAUCGGCAGCAGGACAU |
| exon_4 | | | TACAA (SEQ ID NO: 111) | ACAA (SEQ ID NO: 274) |
| TRAC_ | − | CTTC | CAAATCATTTTAATGAAGGCATCGG | CAAAUCAUUUUAAUGAAGGCAUCGGC |
| exon_4 | | | CAGCA (SEQ ID NO: 112) | AGCA (SEQ ID NO: 275) |
| TRAC_ | + | CTTC | ATTAAAATGATTTGGAAGAGCAGA | AUUAAAAUGAUUUGGAAGAGCAGA |
| exon_4 | | | (SEQ ID NO: 113) | (SEQ ID NO: 276) |
| TRAC_ | + | ATTA | TCGAGGCCCTGCTATGCTGTGTATC | UCGAGGCCCUGCUAUGCUGUGUAUCU |
| exon_4 | | | TGGGC (SEQ ID NO: 114) | GGGC (SEQ ID NO: 277) |
| TRAC_ | + | ATTA | TCTCATTATCGAGGCCCTGCTATGC | UCUCAUUAUCGAGGCCCUGCUAUGCU |
| exon_4 | | | TGTGT (SEQ ID NO: 115) | GUGU (SEQ ID NU; 278) |
| TRAC_ | − | TTTC | TTTATGAACACTATTAAAAAAAAAT | UUUAUGAACACUAUUAAAAAAAAADA |
| exon_4 | | | AAACC (SEQ ID NO: 116) | AACC (SEQ ID NO: 279) |
| TRAC_ | − | TTTT | AATGAAGGCATCGGCAGCAGGACAT | AAUGAAGGCAUCGGCAGCAGGACAUA |
| exon_4 | | | ACAAC (SEQ ID NO: 117) | CAAC (SEQ ID NO: 280) |
| TRAC_ | − | CTTT | ATGAACACTATTAAAAAAAAATAAA | AUGAACACUAUUAAAAAAAAAUAAAC |
| exon_4 | | | CCCCT (SEQ ID NO: 118) | CCCU (SEQ ID NO: 281) |
| TRAC_ | − | ATTG | CCGGGGGGGCACAGAGGTGGTAGCA | CCGGGGGGGCACAGAGGUGGUAGCAG |
| exon_4 | | | GCTTT (SEQ ID NO: 119) | CUUU (SEQ ID NO: 282) |
| TRAC_ | − | ATTA | AAAAAAAATAAACCCCTCACAACAT | AAAAAAAAUAAACCCCUCACAACAUU |
| exon_4 | | | TCTGC (SEQ ID NO: 120) | CUGC (SEQ ID NO: 283) |
| TRAC_ | − | TTTG | GAGAGGGAGAAGAGGGGCAATGCAG | GAGAGGGAGAAGAGGGGCAAUGCAGA |
| exon_4 | | | AGGAA (SEQ ID NO: 121) | GGAA (SEQ ID NO: 284) |
| TRAC_ | − | GTTT | GGAGAGGGAGAAGAGGGGCAATGCA | GGAGAGGGAGAAGAGGGGCAAUGCAG |
| exon_4 | | | GAGGA (SEQ ID NO: 122) | AGGA (SEQ ID NO: 285) |
| TRAC_ | − | GTTC | CCTCTGTTTGGAGAGGGAGAAGAGG | CCUCUGUUUGGAGAGGGAGAAGAGGG |
| exon_4 | | | GGCAA (SEQ ID NO: 123) | CCAA (SEQ ID NO: 285) |
| TRAC_ | − | CTTG | GGGGTAGGAGAGTTCCCTCTGTTTG | GGGGUAGGAGAGUUCCCUCUGUUUGG |
| exon_4 | | | GAGAG (SEQ ID NO: 124) | AGAG (SEQ ID NO: 287) |
| TRAC_ | − | TTTC | ACCTCCTTGGGGGTAGGAGAGTTCC | ACCUCCUUGGGGGUAGGAGAGUUCCC |
| exon_4 | | | CTCTG (SEQ ID NO: 125) | UCUG (SEQ ID NO: 288) |
| TRAC_ | − | CTTT | CACCTCCTTGGGGGTAGGAGAGTTC | CACCUCCUUGGGGGUAGGAGAGUUCC |
| exon_4 | | | CCTCT (SEQ ID NO: 126) | CUCU (SEQ ID NO: 289) |
| TRAC_ | + | CTTC | TTCTCAAGACGTGGGGGGAAATTAT | UUCUCAAGACGUGGGGGGAAAUUAUC |
| exon_4 | | | CTCAT (SEQ ID NO: 127) | UCAU (SEQ ID NO: 290) |
| TRAC_ | − | GTTG | GTGGCATTGCCGGGGGGGCACAGAG | GUGGCAUUGCCGGGGGGGCACAGAGG |
| exon_4 | | | GTGGT (SEQ ID NO: 128) | UGGU (SEQ ID NO: 291) |
| TRAC_ | − | ATTC | GGGTAGGATCCAGTTGGTGGCATTG | GGGUAGGAUCCAGUUGGUGGCAUUGC |
| exon_4 | | | CCGGG (SEQ ID NO: 129) | CGGG (SEQ ID NO: 292) |
| TRAC_ | − | CTTA | ATCATAAATTCGGGTAGGATCCAGT | AUCADAAAUUCGGGUAGGAUCCAGUU |
| exon_4 | | | TGGTG (SEQ ID NO: 130) | GGUG (SEQ ID NO: 293) |
| TRAC_ | − | CTTC | AGCAATCTTAATCATAAATTCGGGT | AGCAAUCUUAAUCAUAAAUUCGGGUA |
| exon_4 | | | AGGAT (SEQ ID NO: 131) | GGAU (SEQ ID NO: 294) |
| TRAC_ | − | TTTG | GCAGCTCTTCAGCAATCTTAATCAT | GCAGCUCUUCAGCAAUCUUAAUCAUA |
| exon_4 | | | AAATT (SEQ ID NO: 132) | AAUU (SEQ ID NO: 295) |
| TRAC_ | − | GTTT | GGCAGCTCTTCAGCAATCTTAATCA | GGCAGCUCUUCAGCAAUCUUAAUCAU |
| exon_4 | | | TAAAT (SEQ ID NO: 133) | AAAU (SEQ ID NO: 296) |
| TRAC_ | − | CTTG | CCTCTGCCGTGAATGTCAGGCAGTG | CCUCUGCCGUGAAUGUCAGGCAGUGA |
| exon_4 | | | ACAAG (SEQ ID NO: 134) | CAAG (SEQ ID NO: 297) |
| TRAC_ | − | ATTC | TGCAGGACCTAGAGCOCAAGAGAAC | UGCAGGACCUAGAGCCCAAGAGAACC |
| exon_4 | | | CCACT (SEQ ID NO: 135) | CACU (SEQ ID NO: 298) |
| TRAC_ | − | TTTA | TGAACACTATTAAAAAAAAATAAAC | UGAACACUAUUAAAAAAAAAUAAACC |
| exon_4 | | | CCCTC (SEQ ID NO: 136) | CCUC (SEQ ID NO: 299) |
| TRAC_ | + | ATTC | TTCTTCTCAAGACGTGGGGGGAAAT | UUCUUCUCAAGACGUGGGGGGAAAUU |
| exon_4 | | | TATCT (SEQ ID NO: 137) | AUCU (SEQ ID NO: 300) |
| TRAC_ | + | GTTT | ATTTTTTTTTAATAGTGTTCATAAA | AUUUUUUUUUAAUAGUGUUCADAAAG |
| exon_4 | | | GAAAT (SEQ ID NO: 138) | AAAU (SEQ ID NO: 301) |
| TRAC_ | + | TITA | ATAGTGTTCATAAAGAAATACATAG | AUAGUGUUCAUAAAGAAAUACAUAGU |
| exon_4 | | | TATTC (SEQ ID NO: 139) | AUUC (SEQ ID NO: 302) |
| TRAC_ | + | ATTG | CTGAAGAGCTGCCAAACACTGCTGC | CUGAAGAGCUGCCAAACACUGCUGCC |
| exon_4 | | | CACCC (SEQ ID NO: 140) | ACCC (SEQ ID NO: 303) |
| TRAC_ | + | ATTA | AGATTGCTGAAGAGCTGCCAAACAC | AGAUUGCUGAAGAGCUGCCAAACACU |
| exon_4 | | | TGCTG (SEQ ID NO: 141) | GCUG (SEQ ID NO: 304) |
| TRAC_ | + | TTTA | TGATTAAGATTGCTGAAGAGCTGCC | UGAUUAAGAUUGCUGAAGAGCUGCCA |
| exon_4 | | | AAACA (SEQ ID NO: 142) | AACA (SEQ ID NO: 305) |
| TRAC_ | + | ATTT | ATGATTAAGATTGCTGAAGAGCTGC | AUGAUUAAGAUUGCUGAAGAGCUGCC |
| exon_4 | | | CAAAC (SEQ ID NO: 143) | AAAC (SEQ ID NO: 306) |
| TRAC_ | + | CTTC | TCCCTCTCCAAACAGAGGGAACTCT | UCCCUCUCCAAACAGAGGGAACUCUC |
| exon_4 | | | CCTAC (SEQ ID NO: 144) | CUAC (SEQ ID NO: 307) |
| TRAC_ | + | ATTG | CCCCTCTTCTCCCTCTCCAAACAGA | CCCCUCUUCUCCCUCUCCAAACAGAG |
| exon_4 | | | GGGAA (SEQ ID NO: 145) | CGAA (SEQ ID NO: 308) |
| TRAC_ | + | GTTC | CCTTATTGCTGCTTGTCACTGCCTG | CCUUAUUGCUGCUUGUCACUGCCUGA |
| exon_4 | | | ACATT (SEQ ID NO: 146) | CAUU (SEQ ID NO: 309) |
| TRAC_ | + | CTTC | CTCTGCATTGCCCCTCTTCTCCCTC | CUCUGCAUUGCCCCUCUUCUCCCUCU |
| exon_4 | | | TCCAA (SEQ ID NO: 147) | CCAA (SEQ ID NO: 310) |
| TRAC_ | + | ATTC | CAGATCTGCAAGATTGTAAGACAGC | CAGAUCUGCAAGAUUGUAAGACAGCC |
| exon_4 | | | CTGTG (SEQ ID NO: 148) | UGUG (SEQ ID NO: 311) |
| TRAC_ | + | CTTC | CTCATTCCAGATCTGCAAGATTGTA | CUCAUUCCAGAUCUGCAAGAUUGUAA |
| exon_4 | | | AGACA (SEQ ID NO: 149) | GACA (SEQ ID NO: 312) |
| TRAC_ | + | GTTC | TTCCTCATTCCAGATCTGCAAGATT | UUCCUCAUUCCAGAUCUGCAAGAUUG |
| exon_4 | | | GTAAG (SEQ ID NO: 150) | DAAG (SEQ ID NO: 313) |
| TRAC_ | + | CTTC | TGTTCTTCCTCATTCCAGATCTGCA | UGUUCUUCCUCAUUCCAGAUCUGCAA |
| exon_4 | | | AGATT (SEQ ID NO: 151) | GAUU (SEQ ID NO: 314) |
| TRAC_ | + | CTTC | CTCAGTTGTCCAGCCTGACTTCTGT | CUCAGUGUCCAGCCUGACUUCUGUUC |
| exon_4 | | | TCTTCC (SEQ ID NO: 152) | UUCC (SEQ ID NO: 315) |
| TRAC_ | − | CTTA | CAATCTTGCAGATCTGGAATGAGGA | CAAUCUUGCAGAUCUGGAAUGAGGAA |
| exon_4 | | | AGAAC (SEQ ID NO: 153) | GAAC (SEQ ID NO: 316) |
| TRAC_ | + | ATTG | TAAGACAGCCTGTGCTCCCTCGCTC | UAAGACAGCCUGUGCUCCCUCGCUCC |
| exon_4 | | | CTTCC (SEQ ID NO: 154) | UUCC (SEQ ID NO: 317) |
| TRAC_ | + | GTTC | ATAAAGAAATACATAGTATTCTTCT | AUAAAGAAAUACAUAGUAUUCUUCUU |
| exon_4 | | | TCTCA (SEQ ID NO: 155) | CUCA (SEQ ID NO: 318) |
| TRAC_ | + | CTTA | TTGCTGCTTGTCACTGCCTGACATT | UUGCUGCUUGUCACUGCCUGACAUUC |
| exon_4 | | | CACGG (SEQ ID NO: 156) | ACGG (SEQ ID NO: 319) |
| TRAC_ | + | CTTG | TCACTGCCTGACATTCACGGCAGAG | UCACUGCCUGACAUUCACGGCAGAGG |
| exon_4 | | | GCAAG (SEQ ID NO: 157) | CAAG (SEQ ID NO: 320) |
| TRAC_ | + | TTTT | AATAGTGTTCATAAAGAAATACATA | AAUAGUGUUCAUAAAGAAAUACAUAG |
| exon_4 | | | GTATT (SEQ ID NO: 158) | UAUU (SEQ ID NO: 321) |
| TRAC_ | + | TTTT | TAATAGTGTTCATAAAGAAATACAT | UAAUAGUGUUCAUAAAGAAAUACAUA |
| exon_4 | | | AGTAT (SEQ ID NO: 159) | GUAU (SEQ ID NO: 322) |
| TRAC_ | + | TTTT | TTAATAGTGTTCATAAAGAAATACA | QUAAUAGUGUUCAUAAAGAAAUACAU |
| exon_4 | | | TAGTA (SEQ ID NO: 160) | AGUA (SEQ ID NO: 323) |
| TRAC_ | + | TTTT | TTTAATAGTGTTCATAAAGAAATAC | UUUAAJAGUGUUCAUAAAGAAAUACA |
| exon_4 | | | ATAGT (SEQ ID NO: 161) | UAGU (SEQ ID NO: 324) |
| TRAC_ | + | TTTT | TTTTAATAGTGTTCATAAAGAAATA | UUUUAAUAGUGUUCAUAAAGAAAUAC |
| exon_4 | | | CATAG (SEQ ID NO: 162) | AUAG (SEQ ID NO: 325) |
| TRAC_ | + | TTTT | TTTTTAATAGTGTTCATAAAGAAAT | UUUUUAAUAGUGUUCAUAAAGAAAUA |
| exon_4 | | | ACATA (SEQ ID NO: 163) | CAUA (SEQ ID NO: 326) |
| TRAC_ | + | ATTG | CTGCTTGTCACTGCCTGACATTCAC | CUGCUUGUCACUGCCUGACAUUCACG |
| exon_4 | | | GGCAG (SEQ ID NO: 164) | GCAG (SEQ ID NO: 327) |
| TRAC_ | + | ATTT | TTTTTTAATAGTGTTCATAAAGAAA | UUUUUUAAUAGUGUUCAUZAAGAAAU |
| exon_4 | | | TACAT (SEQ ID NO: 165) | ACAU (SEQ ID NO: 328) |
| TRAC_ | + | GTTG | TGAGGGGTTTATTTTTTTTTAATAG | UGAGGGGUUUAUUUUUUUUUAAUAGU |
| exon_4 | | | TGTTC (SEQ ID NO: 166) | GUUC (SEQ ID NO: 329) |
| TRAC_ | + | CTTG | GGCTCTAGGTCCTGCAGAATGTTGT | GGCUCUAGGUCCUGCAGAAUGUUGUG |
| exon_4 | | | GAGGG (SEQ ID NO: 167) | AGGG (SEQ ID NO: 330) |
| TRAC_ | + | GTTC | TCTTGGGCTCTAGGTOCTGCAGAAT | UCUUGGGCUCUAGGUCCUGCAGAAUG |
| exon_4 | | | GTTGT (SEQ ID NO: 168) | UUGU (SEQ ID NO: 331) |
| TRAC_ | + | CTTC | AGTGGGTTCTCTTGGGCTCTAGGTC | AGUGGGUUCUCUUGGGCUCUAGGUCC |
| exon_4 | | | CTGCA (SEQ ID NO: 169) | UGCA (SEQ ID NO: 332) |
| TRAC_ | + | ATTC | CCTCCTGCTCCCCAGAGACTGCCTC | CCUCCUGCUCCCCAGAGACUGCCUCC |
| exon_4 | | | CGCCA (SEQ ID NO: 170) | GCCA (SEQ ID NO: 333) |
| TRAC_ | + | ATTC | ACGGCAGAGGCAAGGCTGCTGCAGC | ACGGCAGAGGCAAGGCUGCUGCAGCC |
| exon_4 | | | CTCCC (SEQ ID NO: 171) | UCCC (SEQ ID NO: 334) |
| TRAC_ | + | TTTA | TTTTTTTTTAATAGTGTTCATAAAG | UUUUUUUUUAAUAGUGUUCAUAAAGA |
| exon_4 | | | AAATA (SEQ ID NO: 172) | AAUA (SEQ ID NO: 335) |
| TRAC_ | − | CTTG | CAGATCTGGAATGAGGAAGAACAGA | CAGAUCUGGAAUGAGGAAGAACAGAA |
| exon_4 | | | AGTCA (SEQ ID NO: 173) | CUCA (SEQ ID NO: 336) |

The invention includes all combinations of the direct repeats and spacers listed above, consistent with the disclosure herein. In some embodiments, the RNA guide does not consist of the sequence of:

(SEQ ID NO: 344)

AGAAAUCCGUCUUUCAUUGACGGAAGAGCAACAGUGCUGUGGC;

(SEQ ID NO: 345)

AGAAAUCCGUCUUUCAUUGACGGAACAACAGCAUUAUUCCAGA;

(SEQ ID NO: 346)

AGAAAUCCGUCUUUCAUUGACGGGAAACAGGUAAGACAGGGGU;
or

(SEQ ID NO: 347)

AGAAAUCCGUCUUUCAUUGACGGCAGGAGGAGGAUUCGGAACC.

In some embodiments, a spacer sequence described herein comprises a uracil (U). In some embodiments, a spacer sequence described herein comprises a thymine (T). In some embodiments, a spacer sequence according to Table 5 comprises a sequence comprising a thymine in one or more places indicated as uracil in Table 5.

Modifications

The RNA guide may include one or more covalent modifications with respect to a reference sequence, in particular the parent polyribonucleotide, which are included within the scope of this invention.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, C, U or C) or any intervening percentage (e.g., from 1% to 20%, from 1% to 25%, from 1% to 50%, from 1% to 60%, from 1% to 70%. from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages.

Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters. hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some, embodiments, the sequence includes one or more post-transcriptional mollifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA (Rozenski, J. Crain, P. and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-ara-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g. naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu. Z. et al. (2015) RNA editing by ADAR1 marks dsRNA as “self”. Cell Res. 25, 1283-1284, which is incorporated by reference in its entirety.

### Cas12i Polypeptide

In some embodiments, the composition of the present invention includes a Cas12i polypeptide as described in PCT/US2019/022375.

In some embodiments, the composition of the present invention includes a Cas12i2 polypeptide described herein (e.g., a polypeptide comprising SEQ ID NO: 338 and/or encoded by SEQ ID NO: 337). In some embodiments, the Cas12i2 polypeptide comprises at least one RuvC domain.

A nucleic acid sequence encoding the Cas12i2 polypeptide described herein may be substantially identical to a reference nucleic acid sequence, e.g., SEQ ID NO: 337. In some embodiments, the Cas12i2 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 337. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency). See, e.g., Tijssen, “Hybridization with Nucleic Acid Probes. Part I. Theory and Nucleic Acid Preparation” (Laboratory Techniques in Biochemistry and Molecular Biology, Vol 24).

In some embodiments, the Cas12i2 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 337.

In some embodiments, the Cas12i2 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 338.

In some embodiments, the present invention describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 338. Homology or identity can be determined by amino acid sequence alignment, e.g., using a program such as BLAST, ALIGN, or CLUSTAL, as described herein.

Also provided is a Cas12i2 polypeptide of the present invention having enzymatic activity, e.g., nuclease or endonuclease activity, and comprising an amino acid sequence which differs from the amino acid sequences of SEQ ID NO: 338 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i2 polypeptide comprises a polypeptide having a sequence of SEQ ID NO: 348, SEQ ID NO: 349, SEQ ID NO: 350, SEQ ID NO: 351, or SEQ ID NO: 352.

In some embodiments, the Cas12i2 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 348, SEQ ID NO:349, SEQ ID NO: 350, SEQ ID NO: 351, or SEQ ID NO: 352. In some embodiments, a Cas12i2 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 348, SEQ ID NO:349, SEQ ID NO: 350, SEQ ID NO: 351, or SEQ ID NO: 352 maintains the amino acid changes (or at least 1, 2, 3 etc. of these changes) that differentiate it from its respective parent/reference sequence.

In some embodiments, the present invention describes a Cas12i2 polypeptide having a specified degree of anno acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 348, SEQ ID NO: 349, SEQ ID NO: 350, SEQ ID NO: 351, or SEQ ID NO: 352. Homology or identity can be determined by amino acid sequence alignment, e.g., using a program such as BLAST, ALIGN, or CLUSTAL, as described herein.

Also provided is a Cas12i2 polypeptide of the present invention having enzymatic activity, e.g., nuclease or endonuclease activity, and comprising an amino acid sequence which differs from the amino acid sequences of SEQ ID NO: 348, SEQ ID NO:349, SEQ ID NO: 350, SEQ ID NO: 351, or SEQ ID NO: 352 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present invention includes a Cas12i4 polypeptide described herein (e.g., a polypeptide comprising SEQ ID NO: 354 and/or encoded by SEQ ID NO: 353) In some embodiments, the Cas12i4 polypeptide comprises at least ore RuvC domain.

A nucleic acid sequence encoding the Cas12i4 polypeptide described herein may be substantially identical to a reference nucleic acid sequence, e.g., SEQ ID NO: 353. In some embodiments, the Cas12i4 polypeptide is encoded by a nucleic acid comprising a sequence having least about 60%, at least about 65%. at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 353. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency).

In some embodiments, the Cas12i4 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 353.

In some embodiments, the Cas12i4 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 354.

In some embodiments, the present invention describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 354. Homology or identity can be determined by amino acid sequence alignment, e.g., using a program such as BLAST, ALIGN, or CLUSTAL, as described herein.

Also provided is a Cas12i4 polypeptide of the present invention having enzymatic activity, e.g., nuclease or endonuclease activity, and comprising an amino acid sequence which differs from the amino acid sequences of SEQ ID NO: 354 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i4 polypeptide comprises a polypeptide having a sequence of SEQ NO: 355 or SEQ ID NO: 356.

In some embodiments, the Cas12i4 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 355 or SEQ ID NO: 356. In some embodiments, a Cas12i4 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 355 or SEQ ID NO: 356 maintains the amino acid changes (or at least 1, 2, 3 etc. of these changes) that differentiate it from its respective parent/reference sequence.

In some embodiments, the present invention describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 355 or SEQ ID NO: 356. Homology or identity can be determined by amino acid sequence alignment, e.g., using a program such as BLAST. ALIGN, or CLUSTAL, as described herein.

Also provided is a Cas12i4 polypeptide of the present invention having enzymatic activity, e.g., nuclease or endonuclease activity, and comprising an amino acid sequence which differs from the amino acid sequences of SEQ ID NO: 355 or SEQ ID NO: 356 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present invention includes a Cas12i1 polypeptide described herein (e.g., a polypeptide comprising SEQ ID NO: 357). In some embodiments, the Cas12i4 polypeptide comprises at least one RuvC domain.

In some embodiments, the Cas12i1 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 357.

In some embodiments, the present invention describes a Cas12i1 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 357. Homology or identity can be determined by amino acid sequence alignment, e.g., using a program such as BLAST, ALIGN, or CLUSTAL, as described herein.

Also provided is a Cas12i1 polypeptide of the present invention having enzymatic activity, e.g., nuclease or endonuclease activity, and comprising an amino acid sequence which differs from the amino acid sequences of SEQ ID NO: 357 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present invention includes a Cas12i3 polypeptide described herein (e.g., a polypeptide comprising SEQ ID NO: 358). In some embodiments, the Cas12i4 polypeptide comprises at least one RuvC domain.

In some embodiments, the Cas12i3 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 358.

In some embodiments, the present invention describes a Cas12i3 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 358. Homology or identity can be determined by amino acid sequence alignment, e.g., using a program such as BLAST, ALIGN, or CLUSTAL, as described herein.

Also provided is a Cas12i3 polypeptide of the present invention having enzymatic activity, e.g., nuclease or endonuclease activity, and comprising an amino acid sequence which differs from the amino acid sequences of SEQ ID NO: 358 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

Although the changes described herein may be one or more amino acid changes, changes to the Cas12i polypeptide may also be of a substantive nature, such as fusion of polypeptides as amino- and/or carboxyl-terminal extensions. For example, the Cas12i polypeptide may contain additional peptides, e.g., one or more peptides. Examples of additional peptides may include epitope peptides for labelling, such as a polyhistidine tag (His-tag), Myc, and FLAG. In some embodiments, the Cas12i polypeptide described herein can be fused to a detectable moiety such as a fluorescent protein (e.g., green fluorescent protein (GI-P) or yellow fluorescent protein (YFP)).

In some embodiments, the Cas12i polypeptide comprises at least one (e.g., two, three, four, five, six, or more) nuclear localization signal (NLS). In some embodiments, the Cas12i polypeptide comprises at least one (e.g., two, three, four, five, six, or more) nuclear export signal (NES). In some embodiments, the Cas12i polypeptide comprises at least one (e.g., two, three, four, five, six, or more) NLS and at least one (e.g., two, three, four, five, six, or more) NES.

In some embodiments, the Cas12i polypeptide described herein can be self-inactivating. See, Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the Cas12i polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus. PA).

### Target Sequence

In some embodiments, the target sequence is within a TRAC gene or a locus of a TRAC gene. In some embodiments, the TRAC gene is a mammalian gene. In some embodiments, the TRAC gene is a human gene. For example, in some embodiments, the target sequence is within the sequence of SEQ ID NO: 339 or the reverse complement thereof. In some embodiments, the target sequence is within an exon of the TRAC gene set forth in SEQ ID NO: 339 (or the reverse complement thereof), e.g., within a sequence of SEQ ID NO: 340, 341, 342, or 343 (or a reverse complement thereof). Target sequences within an exon of the TRAC gene of SEQ ID NO: 339 (and the reverse complement thereof) are set forth in Table 5. In some embodiments, the target sequence is within an intron of the TRAC gene set forth in SEQ ID NO: 339 or the reverse complement thereof. In some embodiments, the target sequence is within a variant (e.g., a polymorphic variant) of the TRAC gene sequence set forth in SEQ ID NO: 339 or the reverse complement thereof. In some embodiments, the TRAC gene sequence is a homolog of the sequence set forth in SEQ ID NO: 339 or the reverse complement thereof. For examples, in some embodiments, the TRAC gene sequence is a non-human TRAC sequence.

In some embodiments, the target sequence is adjacent to a 5′-NTTN-3′ PAM sequence, wherein N is any nucleotide. The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-TTY-3′, 5′-DTTR′3′, 5′-CTTR-3′, 5′-DTTT-3′, 5′-ATTN-3′, or 5′-GTTN-3′, wherein Y is C or T. B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-T=−3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some embodiments, the RNA guide binds to a first strand of a double-stranded target sequence (e.g., the target strand or the spacer-complementary strand), and the 5′-NTTN-3′ PAM sequence is present in the second, complementary strand (e.g., the non-target strand or the non-spacer-complementary strand). In some embodiments, the RNA guide binds adjacent to a 5′-NAAN-3′ sequence on the target strand (e.g., the spacer-complementary strand).

In some embodiments, the target sequence is single-stranded (e.g., single-stranded DNA). In some embodiments, the target sequence is double-stranded (e.g., double-stranded DNA). In some embodiments, the target sequence comprises both single-stranded and double-stranded regions. In some embodiments, the target sequence is linear. In some embodiments, the target sequence is circular. In some embodiments, the target sequence comprises one, or more modified nucleotides, such as methylated nucleotides, damaged nucleotides, or nucleotides analogs. In some embodiments, the target sequence is not modified.

In some embodiments, the target sequence is present in a cell. In some embodiments, the target sequence is present in the nucleus of the cell. In some embodiments, the target sequence is endogenous to the cell. In some embodiments, the target sequence is a genomic DNA. In some embodiments, the target sequence is a chromosomal DNA. In some embodiments, the target sequence is a protein-coding gene or a functional region thereof, such as a coding region, or a regulatory element, such as a promoter, enhancer, a 5′ or 3′ untranslated region, etc. In some embodiments, the target sequence is a plasmid.

In some embodiments, the target sequence is present in a readily accessible region of the target sequence. In some embodiments, the target sequence is in an exon of a target gene. In some embodiments, the target sequence is across an exon-intron junction of a target gene. In some embodiments, the target sequence is present in a non-coding region, such as a regulatory region of a gene. In some embodiments, wherein the target sequence is exogenous to a cell, the target sequence comprises a sequence that is not found in the genome of the cell.

In some embodiments, the target sequence is exogenous to a cell. In some embodiments, the target sequence is a horizontally transferred plasmid. In some embodiments, the target sequence is integrated in the genome of the cell. In some embodiments, the target sequence is not integrated in the genome of the cell. In some embodiments, the target sequence is a plasmid in the cell. In some embodiments, the target sequence is present in an extrachromosomal array.

In some embodiments, the target sequence is an isolated nucleic acid, such as an isolated DNA or an isolated RNA. In some embodiments, the target sequence is present in a cell-free environment. In some embodiments, the target sequence is an isolated vector, such as a plasmid. In some embodiments, the target sequence is an ultrapure plasmid.

The target sequence is a locus of the TRAC gene that hybridizes to the RNA guide. In some embodiments, a cell has only one copy of the target sequence. In some embodiments, a cell has more than one copy, such as at least about any one of 2, 3, 4, 5, 10, 100, or more copies of the target sequence.

In some embodiments, a TRAC target sequence is selected to be edited by a Cas12i polypeptide and an RINA guide using one or more of the following criteria. First, in some embodiments, a target sequence near the 5′ end of the TRAC coding sequence is selected. For example, in some embodiments, an RNA guide is designed to target a sequence in exon 1 (SEQ ID NO: 340) or exon 2 (SEQ ID NO: 341).

Second, in some embodiments, a target sequence adjacent to a 5′-CTTY-3′ PAM sequence is selected. For example, in some embodiments, an RNA guide is designed to target a sequence adjacent to a 5′-CTTT-3′ or 5′-CTTC-3′ sequence. Third, in some embodiments, a target sequence having low sequence similarity to other genomic sequences is selected. For example, for each target sequence, potential non-target sites can be identified by searching for other genomic sequences adjacent to a PAM sequence and calculating the Levenshtein distance between the target sequence and the PAM-adjacent sequences. The Levenshtein distance (e.g., edit distance) corresponds to the minimum number of edits (e.g., insertions, deletions, or substitutions) required to change one sequence into another (e.g., to change the sequence of a potential non-target locus into the sequence of the on-target locus). Following this analysis, RNA guides are designed for target sequences that do not have potential off-target sequences with a Levenshtein distance of 0 or 1.

### Production

The present invention includes methods for production of the RNA guide, methods for production of the Cas12i polypeptide, and methods for complexing the RNA guide and Cas12i polypeptide.

### RNA Guide

In some embodiments, the RNA guide is made by in vitro transcription of a DNA template. Thus. for example, in some embodiments, the RNA guide is generated by in vitro transcription of a DNA template encoding the RNA guide using an upstream promoter sequence (e.g., a T7 polymerase promoter sequence). In some embodiments, the DNA template encodes multiple RNA guides or the in vitro transcription reaction includes multiple different DNA templates, each encoding a different RNA guide. In some embodiments. the RNA guide is made using chemical synthetic methods. In some embodiments, the RNA guide is made by expressing the RNA guide sequence in cells transfected with a plasmid including sequences that encode the RNA guide. In some embodiments, the plasmid encodes multiple different RNA guides. In some embodiments, multiple different plasmids, each encoding a different RNA guide, are transfected into the cells. In some embodiments, the RNA guide is expressed from a plasmid that encodes the RNA guide and also encodes a Cas12i polypeptide. In some embodiments, the RNA guide is expressed from a plasmid that expresses the RNA guide but not a Cas12i polypeptide. In some embodiments, the RNA guide is purchased from a commercial vendor. In some embodiments, the RNA guide is synthesized using one or more modified nucleotide, e.g., as described above.

### Cas12i Polypeptide

In some embodiments, the Cas12i polypeptide of the present invention can be prepared by (a) culturing bacteria which produce the Cas12i polypeptide of the present invention, isolating the Cas12i polypeptide, optionally, purifying the Cas12i polypeptide, and complexing the Cas12i polypeptide with an RNA guide. The Cas12i polypeptide can be also prepared by (h) a known genetic engineering technique, specifically, by isolating a gene encoding the Cas12i polypeptide of the present invention from bacteria, constructing a recombinant expression vector, and then transferring the vector into an appropriate host cell that expresses the RNA guide for expression of a recombinant protein that complexes with the RNA guide in the host cell. Alternatively, the Cas12i polypeptide can be prepared by (c) an in vitro coupled transcription-translation system and then complexing with an RNA guide.

In some embodiments, a host cell is used to express the Cas12i polypeptide. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Cas12i polypeptide. After expression of the Cas12i polypeptide, the host cells can be collected and Cas12i polypeptide purified from the cultures etc. according to conventional methods (for example, filtration, centrifugation, cell disruption, gel filtration chromatography, ion exchange chromatography, etc.).

In some embodiments, the methods for Cas12i polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the Cas12i polypeptide. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the Cas12i polypeptide.

A variety of methods can be used to determine the level of production of a Cas12i polypeptide in a host cell. Such methods include, but are not limited to, for example, methods that utilize either polyclonal or monoclonal antibodies specific for the Cas12i polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the Cas12i polypeptide in a cell, comprising providing a polyribonucleotide encoding the Cas12i polypeptide to a host cell wherein the polyribonucleotide encodes the Cas12i polypeptide, expressing the Cas12i polypeptide in the cell, and obtaining the Cas12i polypeptide from the cell.

### Complexing

In some embodiments, an RNA guide targeting TRAC is complexed with a Cas12i polypeptide to form a ribonucleoprotein. In some embodiments, complexation of the RNA guide and Cas12i polypeptide occurs at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the RNA guide does not dissociate from the Cas12i polypeptide at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours.

In some embodiments, the RNA guide and Cas12i polypeptide are complexed in a complexation buffer. In some embodiments, the Cas12i polypeptide is stored in a buffer that is replaced with a complexation buffer to form a complex with the RNA guide. In some embodiments, the Cas12i polypeptide is stored in a complexation buffer.

In some embodiments, the complexation buffer has a pH in a range of about 7.3 to 8.6. In one embodiment, the pH of the complexation buffer is about 7.3. In one embodiment, the pH of the complexation buffer is about 7.4. In one embodiment, the pH of the complexation buffer is about 7.5. In one embodiment, the pH of the complexation buffer is about 7.6. In one embodiment, the pH of the complexation buffer is about 7.7. In one embodiment, the pH of the complexation buffer is about 7.8. In one embodiment, the pH of the complexation buffer is about 7.9. In one embodiment, the pH of the complexation buffer is about 8.0. In one embodiment, the pH of the complexation buffer is about 8.1. In one embodiment, the pH of the complexation buffer is about 8.2. In one embodiment, the pH of the complexation buffer is about 8.3. In one embodiment, the pH of the complexation buffer is about 8.4. In one embodiment, the pH of the complexation buffer is about 8.5. In one embodiment, the pH of the complexation buffer is about 8.6.

In some embodiments, the Cas12i polypeptide can be overexpressed and complexed with the RNA guide in a host cell prior to purification as described herein. In some embodiments, mRNA or DNA encoding the Cas12i polypeptide is introduced into a cell so that the Cas12i polypeptide is expressed in the cell. In some embodiments, the RNA guide is also introduced into the cell, whether simultaneously. separately, or sequentially from a single mRNA or DNA construct, such that the ribonucleoprotein complex is formed in the cell.

### Delivery

Compositions or complexes described herein may be formulated, for example, including a carrier. such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers): electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the Cas12i polypeptide, RNA guide, donor DNA, etc.), one or more transcripts thereof, and/or a pre-formed RNA guide/Cas12i polypeptide complex to a cell, where a ternary complex is formed. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine): non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnetofection or magnet assisted transfection, particle bombardment; and hybrid methods, such as nucleofection. In some embodiments, the present application further provides cells produced by such methods, and organisms (such as animals, plants, or fungi) comprising or produced from such cells.

In some embodiments, the Cas 2i component and the RNA guide component are delivered together. For example, in some embodiments, the Cas12i component and the RNA guide component are packaged together in a single AAV particle. In another example, in some embodiments, the Cas 12i component and the RNA guide component are delivered together via lipid nanoparticles (LNPs). In some embodiments, the Cas12i component and the RNA guide component are delivered separately. For example, in some embodiments, the Cas2i component and the RNA guide are packaged into separate AAV particles. In another example, in some embodiments, the Cas12i component is delivered by a first delivery mechanism and the RNA guide is delivered by a second delivery mechanism.

### Cells

Compositions or complexes described herein can be delivered to a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte). a bone cell (e.g., an osteoblast, osteocyte, osteoclast), a blood cell (e.g., a monocyte, a lymphocyte, a neutrophil, an cosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments. the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or a murine cell. In some embodiments, the murine cell is derived from a wild-type mouse, an immunosuppressed mouse, or a disease-specific mouse model. In some embodiments, the cell is a cell within a living tissue, organ, or organism.

### Methods

The disclosure also provides methods of modifying a target sequence within the TRAC gene. In some embodiments, the methods comprise introducing a TRAC-targeting RNA guide and a Cas12i polypeptide into a cell. The TRAC-targeting RNA guide and Cas12i polypeptide can be introduced as a ribonucleoprotein complex into a cell. The TRAC-targeting RNA guide and Cas12i polypeptide can be introduced on a nucleic acid vector. The Cas12i polypeptide can be introduced as an mRNA. The RNA guide can be introduced directly into the cell.

In some embodiments, the sequence of the TRAC gene is set forth in SEQ ID NO: 339 or the reverse complement thereof. In some embodiments, the target sequence is in an exon of a TRAC gene, such as an exon having a sequence set forth m any one of SEQ ID NO: 340, SEQ ID NO: 341, SEQ ID NO: 342, or SEQ ID NO: 343, or a reverse complement thereof. In some embodiments, the target sequence is in an intron of a TRAC gene (e.g., an intron of the sequence set forth in SEQ ID NO: 339 or the reverse complement thereof). In other embodiments, the sequence of the TRAC gene is a variant of the sequence set forth in SEQ ID NO: 339 (or the reverse complement thereof) or a homolog of the sequence set forth in SEQ ID NO: 339 (or the reverse complement thereof). For example, in some embodiments, the target sequence is polymorphic variant of the TRAC sequence set forth in SEQ ID NO: 339 (or the reverse complement thereof) or a non-human form of the TRAC gene.

In some embodiments, an RNA guide as disclosed herein is designed to be complementary to a target sequence that is adjacent to a 5′-NTTN-3′ PAM sequence. The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′. In some embodiments, the RNA guide is designed to bind to a first strand of a double-stranded target sequence (e.g., the target strand or the spacer-complementary strand), and the 5′-NTTN-3′ PAM sequence is present in the second, complementary strand (e.g., the non-target strand or the non-spacer-complementary strand). In some embodiments, the RNA guide binds adjacent to a 5′-NAAN-3′ sequence on the target strand (e.g., the spacer-complementary strand).

In some embodiments, the Cas12i polypeptide has enzymatic activity (e.g., nuclease activity). In some embodiments, the Cas12i polypeptide induces one or more DNA double-stranded breaks in the cell. In some embodiments, the Cas12i polypeptide induces one or more DNA single-stranded breaks in the cell. In some embodiments, the Cas12i polypeptide induces one or more DNA nicks in the cell. In some embodiments, DNA breaks and/or nicks result in formation of one or more indels (e.g., one or more deletions).

In some embodiments, an RNA guide disclosed herein forms a complex with the Cas12i polypeptide and directs the Cas12i polypeptide to a target sequence adjacent to a 5′-NTTN-3′ sequence. In some embodiments, the complex induces a deletion (e.g., a nucleotide deletion or DNA deletion) adjacent to the 5′-NTTN-3′ sequence. In some embodiments, the complex induces a deletion adjacent to a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the complex induces a deletion adjacent to a T/C-rich sequence.

In some embodiments, the deletion is downstream of a 5′-NTTN-3′ sequence. In some embodiments, the deletion is downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion is downstream of a T/C-rich sequence.

In some embodiments, the deletion alters expression of the TRAC gene. In some embodiments, the deletion alters function of the TRAC gene. In some embodiments, the deletion inactivates the TRAC gene. In some embodiments, the deletion is a frameshifting deletion. In some embodiments, the deletion is a non-frameshifting deletion. In some embodiments, the deletion leads to cell toxicity or cell death (e.g., apoptosis).

In some embodiments, the deletion starts within about 5 to about 15 nucleosides (e.g., about 3, 4, 5, 6, 7, 8, 9, 10, 11, 12, 13, 14, 15, 16, or 17 nucleotides) of the 5′-NTTN-3′ sequence. In some embodiments, the deletion starts within about 5 to about 15 nucleotides (e.g., about 3, 4, 5, 6, 7, 8, 9, 10, I1, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence.

In some embodiments, the deletion starts within about 5 to about 10 nucleotides (e.g., about 3, 4, 5, 6, 7, 8, 9, 10, 11, or 12 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5-TTTC-3, 5′-GTTA-3′, 5′-GTTT-3′, 5′-GTTC-3′, 5′-GTTG-3′, 5′-CTTA-3′, 5′-CTTT-3′, 5′-CTTG-3′, or 5′-CTTC-3′ sequence. In some embodiments, the deletion starts within about to about 10 nucleotides (e.g., about 3, 4, 5, 6, 7, 8, 9, 10, 11, or 12 nucleotides) downstream of a T/C-rich sequence.

In some embodiments, the deletion starts within about 10 to about 15 nucleotides (e.g., about 8, 9, 10, 11, 12, 13, 14, 15, 16, or 17 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 1, 12, 13, 14, 15, 16, or 17 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a T/C-rich sequence.

In some embodiments, the deletion ends within about 20 to about 25 nucleotides (e.g., about 17, 18, 19.20, 21, 22, 23, 24.25, 26, 27, or 28 nucleotides) of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-ATTA-3′, 5′-ATTT-3′, 5′-ATTG-3′, 5′-ATTC-3′, 5′-TTTA-3′, 5′-TT-3′, 5′-TTTG-3′, 5′-TTTC-3′, 5′-GTTA-3′, 5′-GT-3′, 5′-GTTG-3′, 5′-GTTC-3′, 5′-CTTA-3′, 5′-CTTT-3′, 5′-CTTG-3′, or 5′-GTTC-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 1, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a TIC-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-TTTG-3′, 5′-GTTC-3′, 5′-GTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 15 nucleotides (e.g., about 34, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATT-3′, 5′ ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments. the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTC-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments. the deletion start, within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about to about 10 nucleotides and ends within about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′ ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about to about 10 nucleotides (e.g., about 3, 4, 5, 6, 7, 8, 9, 10, 11, or 12 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about to about 10 nucleotides (e.g., about 3, 4, 5, 6, 7, 8, 9, 10, 11, or 12 nucleotides) downstream of a TIC-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g, about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-CTTT-3′, 5′-TTTG-3′, 5′-GTTC-3′, 5′-CTTA-3′, 5′-CTTT-3′, 5′-CTTG-3′, or 5′-CTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTA-3′, 5′-CTTT-3′, 5′-CTTG-3′, or 5′-TTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion is up to about 50 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides). In some embodiments, the deletion is up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides) In some embodiments, the deletion is between about 10 nucleotides and about 15 nucleotides in length (e.g., about 7, 8, 9, 10, 11, 12, 13, 14, 15, 16, or 17 nucleotides).

In some embodiments, the methods described herein are used to engineer a cell comprising a deletion as described herein in a TRAC gene.

Compositions, vectors, nucleic acids, RNA guides and cells disclosed herein may be used in therapy Compositions, vectors, nucleic acids. RNA guides and cells disclosed herein may be used in methods of treating a disease or condition in a subject. Any suitable delivery or administration method known in the art may be used to deliver compositions, vectors, nucleic acids, RNA guides and cells disclosed herein. Such methods may involve contacting a target sequence with a composition, vector, nucleic acid, or RNA guide disclosed herein. Such methods may involve a method of editing a TRAC sequence as disclosed herein. In some embodiments, a cell engineered using an RNA guide disclosed herein is used for ex vivo gene therapy. In some embodiments, a cell engineered using an RNA guide disclosed herein is used for CAR T-cell therapy.

### KITS

The invention also provides kits or systems that can be used, for example, to carry out a method described herein. In some embodiments, the kits or systems include an RNA guide and a Cas12i polypeptide. In some embodiments, the kits or systems include a polynucleotide that encodes such a Cas12i polypeptide, and optionally the polynucleotide is comprised within a vector, e.g., as described herein. In some embodiments, the kits or systems include a polynucleotide that encodes an RNA guide disclosed herein. The Cas12i polypeptide and the RNA guide (e.g., as a ribonucleoprotein) can be packaged within the same or other vessel within a kit or system or can be packaged in separate vials or other vessels, the contents of which can be mixed prior to use. The kits or systems can additionally include, optionally, a buffer and/or instructions for use of the RNA guide and Cas12i polypeptide.

All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Editing of TRAC in a Mammalian Cell Via Transfection

This Example describes indel assessment on multiple TRAC targets using Cas12i2 and RNA guide compositions introduced into mammalian cells by transient transfection.

Variant Cas12i2 of SEQ ID NO: 348 was cloned with a CMV promotor in a peda3.1 backbone (Invitrogen). The plasmids were then maxi-prepped and diluted to 1 μg/μL. For RNA guide preparation, a dsDNA fragment encoding an RNA guide was derived by ultramers containing the target sequence scaffold, and the U6 promoter. Ultramers were resuspended in 10 mM Tris·HCl at a pH of 7.5 to a final stock concentration of 100 μM. Working stocks were subsequently diluted to 10 μM, again using 10 mM Tris·HCl to serve as the template for the PCR reaction. The amplification of the RNA guide was done in 50 μL reactions with the following components: 0.02 μl of aforementioned template, 2.5 μl forward primer, 2.5 μl reverse primer, 25 μL NEB HiFi Polymerase, and 20 μl water. Cycling conditions were: 1×(30 s at 98° C.), 30×(10 s at 98° C. 15 s at 67° C.), 1×(2 min at 72° C.). PCR products were cleaned up with a 1.8×SPRI treatment and normalized to 25 ng/μL. The prepared RNA guide sequences and their corresponding target sequences are shown in Table 6.

TABLE 6
RNA guide and Target Sequences for Transient Transfection.

| Target | RNA Guide | Target Sequence |
|---|---|---|
| TRAC_exon1_target | AGAAAUCCGUCUUUCAUUGACGGUG | TGGATATCTGTGGGACAA |
| 1 | GAUAUCUGUGGGACAAGA (SEQ | GA (SEQ ID NO: 398) |
| | ID NO: 385) | |
| TRAC_exon1_target | AGAAAUCCGUCUUUCAUUGACGGGU | GTGACACATTTGTTTGAG |
| 2 | GACACAUUUGUUUGAGAA (SEQ | AA (SEQ ID NO: 399) |
| | ID NO: 386) | |
| TRAC exon1_target | AGAAAUCCGUCUUUCAUUGACGGAA | AAGAGCAACAGTGCTGTG |
| 3 | GAGCAACAGUGCUGUGGC (SEQ | GC (SEQ ID NO: 400) |
| | ID NO: 344) | |
| TRAC_exon1_target | AGAAAUCCGUCUUUCAUUGACGGGC | GCATGTGCAAACGCCTTC |
| 4 | AUGUGCAAACGCCUUCAA (SEQ | AA (SEQ ID NO: 401) |
| | ID NO: 387) | |
| TRAC_exon1_target | AGAAAUCCGUCUUUCAUUGACGGAA | AACAACAGCATTATTCCA |
| 5 | CAACAGCAUUAUUCCAGA (SEQ | GA (SEQ ID NO: 402) |
| | ID NO: 345) | |
| TRAC_exonl_target | AGAAAUCCGUCUUUCAUUGACGGUG | TGGAATAATGCTGTTGTT |
| 6 | GAAUAAUGCUGUUGUUGA (SEQ | GA (SEQ ID NO: 403) |
| | ID NO: 388) | |
| TRAC exonl_target | AGAAAUCCGUCUUUCAUUGACGGUU | TTCCCCAGCCCAGGTAAG |
| 7 | CCCCAGCCCAGGUAAGGG (SEQ | GG (SEQ ID NO: 404) |
| | ID NO: 389) | |
| TRAC_exon1_target | AGAAAUCCGUCUUUCAUUGACGGCC | CCCAGCCCAGGTAAGGGC |
| 8 | CAGCCCAGGUAAGGGCAG (SEQ | AG (SEQ ID NO: 405) |
| | ID NO: 390) | |
| TRAC_exon2_target | AGAAAUCCGUCUUUCAUUGACGGCU | CTAAAAGGAAAAACAGAC |
| 1 | AAAAGGAAAAACAGACAU (SEQ | AT (SEQ ID NO: 406) |
| | ID NO: 391) | |
| TRAC_exon2_target | AGAAAUCCGUCUUUCAUUGACGGCU | CTGTGATGTCAAGCTGGT |
| 2 | GUGAUGUCAAGCUGGUCG (SEQ | CG (SEQ ID NO: 407) |
| | ID NO: 392) | |
| TRAC exon2_target | AGAAAUCCGUCUUUCAUUGACGGUC | TCTCGACCAGCTTGACAT |
| 3 | UCGACCAGCUUGACAUCA (SEQ | CA (SEQ ID NO: 408) |
| | ID NO: 393) | |
| TRAC exon2_target | AGAAAUCCGUCUUUCAUUGACGGGA | GAAACAGGTAAGACAGGG |
| 4 | AACAGGUAAGACAGGGGU (SEQ | GT (SEQ ID NO: 409) |
| | ID NO: 346) | |
| TRAC_exon3_target | AGAAAUCCGUCUUUCAUUGACGGGU | GTATCTGTAAAACCAAGA |
| 1 | AUCUGUAAAACCAAGAGG (SEQ | GG (SEQ ID NO: 410) |
| | ID NO: 394) | |
| TRAC_exon3_target | AGAAAUCCGUCUUUCAUUGACGGCA | CAAAACCTGTCAGTGATT |
| 2 | AAACCUGUCAGUGAUUGG (SEQ | GG (SEQ ID NO: 411) |
| | ID NO: 395) | |
| TRAC_cxon3_target | AGAAAUCCGUCUUUCAUUGACGGCG | CGAATCCTCCTCCTGAAA |
| 3 | AAUCCUCCUCCUGAAAGU (SEQ | GT (SEQ ID NO: 412) |
| | ID NO: 396) | |
| TRAC_cxon3_target | AGAAAUCCGUCUUUCAUUGACGGCA | CAGGAGGAGGATTOGGAA |
| 4 | GGAGGAGGAUUCGGAACC (SEQ | CC (SEQ ID NO: 413) |
| | ID NO: 347) | |
| TRAC_exon3_target | AGAAAUCCGUCUUUCAUUGACGGAA | AAGGCCCCTCACCTCAGC |
| 5 | GGCCCCUCACCUCAGCUG (SEQ | TG (SEQ ID NO: 414) |
| | ID NO: 397) | |

Approximately 16 hours prior to transfection, 100 μl of 25,000 HEK293T cells in DMEM/10% FBS+Pen/Strep were plated into each well of a 96-well plate. On the day of transfection, the cells were 70-90% confluent. For each well to be transfected, a mixture of 0.5 μl of Lipofectamine 2000 and 9.5 μl of Opti-MEM was prepared and then incubated at room temperature for 5-20 minutes (Solution 1). After incubation, the lipofectamine:OptiMEM mixture was added to a separate mixture containing 182 ng of effector plasmid and 14 ng of RNA guide and water up to 10 μL (Solution 2). The solution 1 and solution 2 mixtures were mixed by pipetting up and down and then incubated at room temperature for 25 minutes. Following incubation, 20 μL of the Solution 1 and Solution 2 mixture were added dropwise to each well of a 96 well plate containing the cells, 72 hours post transfection, cells are trypsinized by adding 10 μL of TrypLE to the center of each well and incubated for approximately 5 minutes, 100 μL of D10 media was then added to each well and mixed to resuspend cells. The cells were then spun down at 500 g for 10 minutes, and the supernatant was discarded. QuickExtract buffer was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled, loaded onto a 2% E-gel EX for 10 minutes and gel extracted. Sequencing runs were done with a 150 cycle NextSeq v2.5 mid or high output kit.

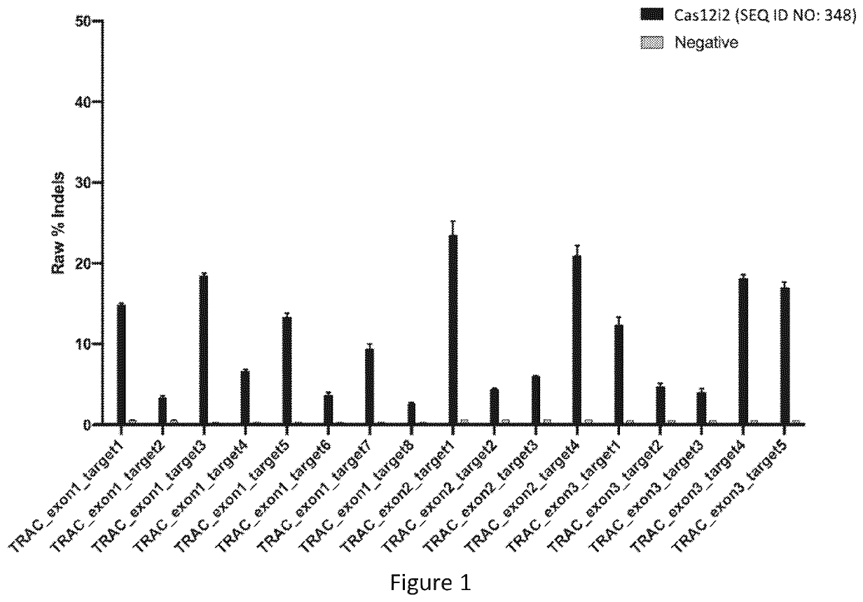

As shown in FIG. 1, each of the eleven tested RNA guides induced indels in TRAC target sequences. Therefore, RNA guides and the variant Cas12i2 of SEQ ID NO: 348 were able to target TRAC targets in exon 1, exon 2, and exon 3 in mammalian cells.

### Example 2—Editing of TRAC In a Mammalian Cell by RNP Electroporation

This Example describes ribonucleoprotein (RNP) transfection followed by FACS staining and indel assessment on multiple TRAC target sequences using a Cas12i polypeptide in mammalian cells.

CD3+ T cells from three individual donors were revived and counted using an automated cell counter. A sample from each donor was collected and stained for CD3ε and DAPI for flow cytometry analysis of surface expression and viability, respectively. Cell density was adjusted to 1e6 cells/mL and cells were stimulated for 3 days with a cocktail of anti-CD3:CD28 antibodies.

Variant Cas12i2 RNP complexation reactions were made by mixing purified variant Cas12i2 (400 μM; SEQ ID NO: 349) with RNA guide i mM in 250 mM NaCl; see sequences in Table 7) at a 1:1 (effector:crRNA) volume ratio (2.5:1 crRNA:effector molar ratio). SpCas9 RNP complexation reactions were made by mixing purified SpCas9 (Aldevron: 62 μM) with sgRNA (1 mM in water; see sequences in Table 7) at a 6.45:1 (effector:sgRNA) volume ratio (2.5:1 sgRNA:effector molar ratio). For “effector only” controls, variant Cas12i2 or SpCas9 were mixed with Protein Storage Buffer (25 mM Tris, pH 7.5, 250 mM NaCl, 1 mM TCEP, 50% glycerol) at the same volume ratio as the crRNA or sgRNA, respectively. Additional controls were included: SpCas9 (Aldevron) with either Lethal #1 (transfection control guide). pooled CD3, or ROSA26 sgRNAs and SpCas9 (Horizon) with either Lethal #1, pooled CD33, or ROSA26 sgRNAs. Complexations were incubated at 37° C. for 30-60 min. Following incubation, RNPs were diluted to 20 μM, 50 μM, 100 μM, or 160 μM effector concentration for variant Cas12i2 and 20 μM or 50 μM for SpCas9.

TABLE 7
RNA guide sequences for RNP transfection.

| | | | | Target | |
|---|---|---|---|---|---|
| Guide Name | Gene | Effector | PAM | Strand | RNA guide |
| Cas12i2_TRAC_ | TRAC | Cas12i2 | CTTC | TS | AGAAAUCCGUCULUCAUUGACGG |
| exon1_target3 | | | | | AAGAGCAACAGUGCUGUGGC |
| | | | | | (SEQ ID NO: 344) |
| Cas12i2_TRAC_ | TRAC | Cas12i2 | CTTC | BS | AGAAAUCCGUCULUCAUUGACGG |
| exon1_target5 | | | | | AACAACAGCAUUAUUCCAGA |
| | | | | | (SEQ ID NO: 345) |
| Cas12i2 TRAC | TRAC | Cas12i2 | CTTT | TS | AGAAAUCCGUCULUCAUUGACGG |
| exon2_target4 | | | | | GAAACAGGUAAGACAGGGGU |
| | | | | | (SEQ ID NO: 346) |
| Cas12i2 TRAC | TRAC | Cas12i2 | CTTT | BS | AGAAAUCCGUCULUCAUUGACGG |
| exon3_target4 | | | | | CAGGAGGAGGAUUCGGAACC |
| | | | | | (SEQ ID NO: 347) |
| SpCas9_TRAC | TRAC | SpCas9 | TGG | BS | mA*mA*mG*UUCCUGUGAUGUCA |
| exon2_target1 | | | | | AGCGUUUUAGAGCUAGAAAUAGC |
| | | | | | AAGUUAAAAUAAGGCUAGUCCGU |
| | | | | | UAUCAACUUGAAAAAGUGGCACC |
| | | | | | GAGUCGGUGCmU*mU*mU*U |
| | | | | | (SEQ ID NO: 384) |

Diluted complexed reactions were dispensed at 2 μL per well into a 384 well electroporation plate. Cell suspensions were collected and counted using an automated cell counter. Cell density was adjusted to 1.1e7 cells/mL in P3 buffer and was dispensed at 2e5 cells/reaction (18 μL). Final concentration of variant Cas12i2 RNPs was 2 μM, 5 μM, 10 μM, or 16 μM. Final concentration of SpCas9 RNPs was 2 or 5 μM. The following controls were set up: unelectroporated cells only, cells in P3 primary cell buffer (Lonza #VXP-3032) only, cells in Protein Storage Buffer only. The plate was electroporated using an electroporation device (program EO-115-AA, Lonza HT), excluding the unelectroporated conditions. Each well was split into four 96-well editing plates (containing 200 μL total volume) using robotics (StarLab Hamilton). Editing plates were incubated for 7 days at 37° C. with 100 NL media replacement at day 4.

After 7 days, plates were spun down and the supernatant was removed. Pellets were resuspended in 200 μL of PBS, 100 μL of sample was collected and stained with either the antibody panel (anti-TRAC) or anti-CD3ε antibody (lethal #1, pooled CD3ε, ROSA26, Protein Storage Buffer and unelectroporated for Cas9 controls). All cells were stained with DAPI to assess viability. Remaining cell suspension was transferred to a 96-well PCR plate and pelleted at 500×g for 5 min. Supernatants were removed and pellets were frozen at −80° C.

For gDNA extraction, pellets were thawed to room temperature and resuspended in appropriate volume of DNA extraction buffer (QuickExtract) to give final concentration of 1000 cells/μL. Samples were then cycled in PCR machine at 65° C. for 15 min, 68° C. for 15 min, 98° C. for 10 min. Samples were then frozen at −20° C.

Samples for Next Generation Sequencing (NGS) were prepared by rounds of PCR. The first round (PCR I) was used to amplify the genomic regions flanking the target site and add NGS adapter %. The second round (PCR H) was used to add NGS indexes. Reactions were then pooled, purified by column purification. and quantified on a fluorometer (Qubit). Sequencing runs were done using a 150 cycle NGS instrument (NextSeq v2.5) mid or high output kit and run on an NGS instrument (NextSeq 550).

For NGS analysis, the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples, the first 30 nt of each read was required to match the reference and reads where over half of the mapping nucleotides were mismatches were filtered out as well. Up to 50,000 reads passing those filters were used for analysis, and reads were counted as an indel read if they contained an insertion or deletion. The indel % was calculated as the number of indel-containing reads divided by the number of reads analyzed (reads passing filters up to 50,000). The QC standard for the minimum number of reads passing filters was 10,000.

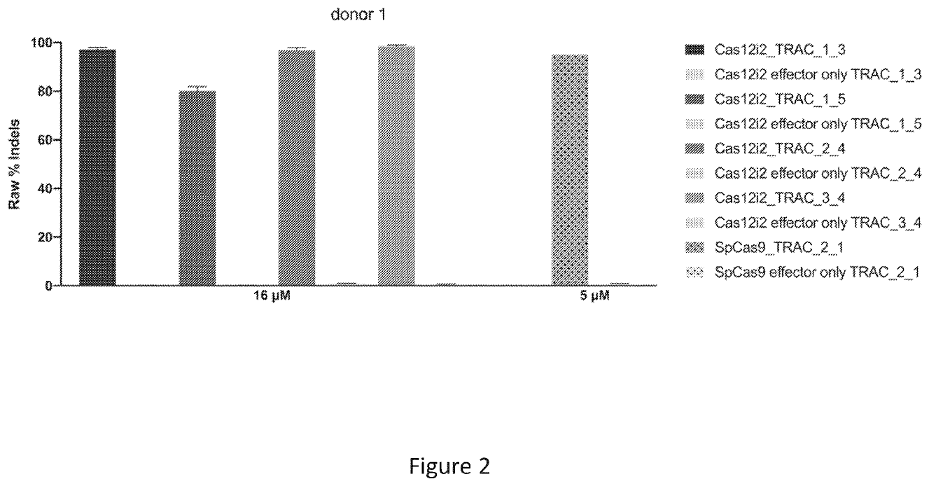

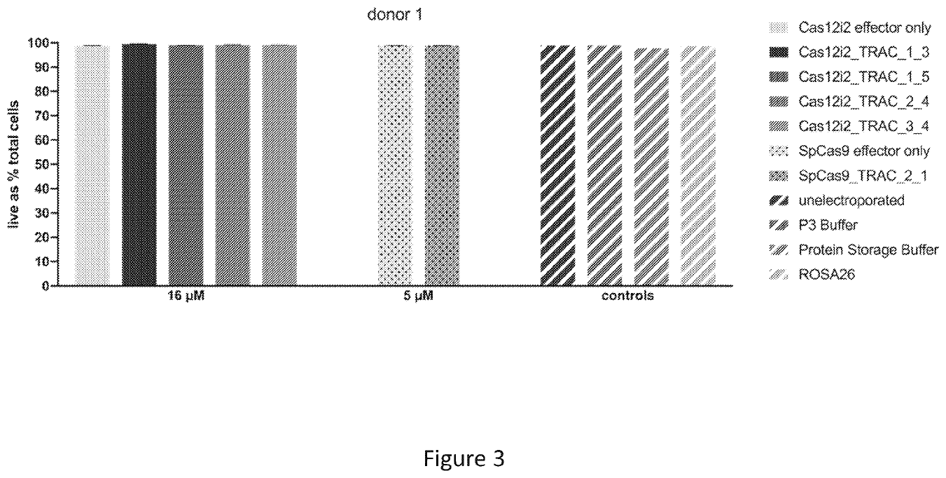

The results showed indels in TRAC induced by variant Cas12i2 RNP targeting in primary T cells (FIG. 2). Cell viability remained high for all conditions seven days post electroporation of the Cas12i2 RNPs targeting TRAC (FIG. 3).

This Example thus shows how to measure viability of cells, e.g., T cells, electroporated with the RNA guide/Cas12i polypeptide complexes described herein, expression of TRAC in the cells, and activity on TRAC target sequences (indel %) in the cells.

This Example further shows that RNA guides and the variant Cas12i2 of SEQ ID NO: 349 were able to target TRAC targets in exon 1, exon 2, and exon 3 in mammalian cells.

| | | |
|---|---|---|
| Nucleotide | atgagcagcg cgatcaaaag ctacaagagc gttctgcgtc cgaacgagcg taagaaccaa | 60 |
| sequence | ctgctgaaaa gcaccattca gtgcctggaa gacggtagcg cgttcttttt caagatgctc | 120 |
| encoding | caaggcctgt ttggtggcat caccccggag attgttcgtt tcagcaccga acaggagaaa | 180 |
| Cas12i2 - | cagcaacagg atatcgcgct gtggtgcgcg gttaactggt tccgtccggt gagccaagac | 240 |
| SEQ ID NO: | agcctgaccc acaccattgc gagcgataac ctggtggaga agtttgagga atactatggt | 300 |
| 337 | ggcaccgcga gcgacgcgat caaacagtac ttcagcgcga gcattggcga aagctactat | 360 |
| | tggaacgact gccgtcaaca gtactatgat ctgtgccgtg agctgggtgt tgaggtgagc | 420 |
| | gacctgaccc atgatctgga gatcctgtgc cgtgaaaagt gcctggcggt tgcgaccgag | 480 |
| | agcaaccaga acaacagcat cattagcgtt ctgtttggca ccggcgaaaa agaggaccgt | 540 |
| | agcgtgaaac tgcgtatcac caagaaaatt ctggaggcga tcagcaacct gaaagaaatc | 600 |
| | ccgaagaacg ttgcgccgat tcaagagatc attctgaacg tggcgaaagc gaccaaggaa | 660 |
| | accttccgtc aggtgtatgc gggtaaccty ggtgcgccga gcaccctgga gaaatttatc | 720 |
| | gcgaaggacg gccaaaaaga gttcgatctg aagaaactgc agaccgacct gaagaaagtt | 780 |
| | attcgtggta aaagcaagga gcgtgattgg tcctgccagg aagagctgcg tagctacgtg | 840 |
| | gagcaaaaca ccatccagta tgacctgtgg gcgtggggcg aaatgttcaa caaagcgcac | 900 |
| | accgcgctga aaatcaagag cacccgtaac tacaactttg cgaagcaacg tctggaacag | 960 |
| | ttcaaagaga ttcagagcct gaacaacctg ctcgttgtga agaagctgaa cgactttttc | 1020 |
| | gatagcgaat ttttcagcgg cgaggaaacc tacaccatct gcgttcacca tctgggtggc | 1080 |
| | aaggacctga gcaaactgta taaggcgtgg gaggatgatc cggcggaccc ggaaaacgcg | 1140 |
| | attgtggttc tgtgcgacga tctgaaaaac aactttaaga aagagccgat ccgtaacatt | 1200 |
| | ctgcgttaca tcttcaccat tcgtcaagaa tgcagcgcgc aggacatcct ggcggcggcg | 1250 |
| | aagtacaacc aacagctgga tcgttataaa agccsaaagg cgaacccgag cgttctgggt | 1320 |
| | aaccagggct ttacctggac caacgcggty atcctgccgg agaaggcgca gcgtaacgac | 1380 |
| | cgsccgaaca gcctggatct gcgtatttyg ctgtacctga aactgcgtca cccggacggt | 1440 |
| | cgttggaaga aacaccatat cccgttctac gatacccgtt tcttccaaga aatttatgcg | 1500 |
| | gcgggcaaca gcccggttga cacctgccag tttcgtaccc cgcgtttcgg ttatcacctg | 1560 |
| | ccgaaactga ccgatcagac cgcqatccgt gttaacaaga aacatqtgaa agcqqcgaag | 1620 |
| | accgaggcgc gtattcgtct ggcgatccaa cagggcaccc tgccggtgag caacctgaag | 1680 |
| | atcaccgaaa ttagcgcgac catcaacagc aaaggtcaag tgcgtattcc gcttaagttt | 1740 |
| | gacgtgggtc gtcaaaaagg caccctgcag atcggtgacc gtttctgcgg ctacgatcaa | 1800 |
| | aaccagaccg cgagccacgc gtatagcctg tgggsagtgg ttaaagaggg tcaataccat | 1860 |
| | asagagctgg gctgctttgt tcgtttcatc agcagcggtg acatcgtgag cattaccgag | 1920 |
| | aaccgtggca accaatttga tcagctgagc tatgaaggtc tcgcgtaccc gcaatatgcc | 1980 |
| | gactggcgta agaaagcgag caagttcgtg agcctgtggc agatcaccaa gaaaaacaag | 2040 |
| | aaaaaggaaa tcgtgaccgt tgaagcgaaa gagaagtttg acgcgatctg caagtaccag | 2100 |
| | ccgcgtctgt ataaattcaa caaggagtac gcgtatctgc tgcgtgatat tgttcgtggc | 2160 |
| | aaaagcctgg tggaactgca acagattcgt caagagatct ttcgtttcat tgaacaggac | 2220 |
| | tgcggtgtta cccgtctggg cagcctgagc ctgagcaccc tggaaaccgt gaaagcggtt | 2280 |
| | aagggtatca tttacagcta ttttagcacc gcgctgaacg cgagcaagaa caacccgatc | 2340 |
| | agcgacgaac agcgtaaaga gtttgatccg gaactgttcg cgctgctgga aaagctggag | 2400 |
| | ctgattcgta cccgtaaaaa gaaacaaaaa gtggaacgta tcgcgaacag cctgattcag | 2460 |
| | acctgcctgg agaacaacat caagttcatt cgtggtgaag gcgacctgag caccaccaac | 2520 |
| | aacgcgacca agaaaaaggc gaacagccgt agcatggatt ggttggcgcg tggtgttttt | 2580 |
| | aacaaaatcc gtcaactggc gccgatgcac aacattaccc tcttcggttg cggcagcctg | 2640 |
| | tacaccagcc accaggaccc gctggtgcat cgtaacccgg ataaagcgat gaagtgccgt | 2700 |
| | tgggcggcga tcccggttaa ggacattggc gattgggtgc tgcgtaagct gagccaaaac | 2760 |
| | ctgcgtgcga aaaacatcgg caccggcgag tactatcacc aaggtgttaa agagttcctg | 2820 |
| | agccattatg aactgcagga cctggaggaa gagctgctga agtggcgtag cgatcgtaaa | 2880 |
| | agcaacattc cgtgctgggt gctgcagaac cgtctggcgg agaagctggg caacaaagaa | 2940 |
| | gcggtggttt acatcccggt tcgtggtggc cgtatttatt ttgcgaccca caaggtggcg | 3000 |
| | accggtgcgg tgagcatcgt tttcgaccaa aaacsagtgt gggtttgcaa cgcggatcat | 3060 |
| | gttgcggcgg cgaacatcgc gctgaccgtg aagggtattg gcgaacaaag cagcgacgaa | 3120 |
| | gagaacccgg atggtagccg tatcaaactg cagctgacca gc | 3162 |

| | |
|---|---|
| Cas12i2 | MSSAIKSYKSVERPNERKNQLLKSTIQCLEDGSAFFFKMLQGLEGGITPEIVRESTEQEKQQQDIAL |
| amino acid | WCAVNWFRPVSQDSLTHTIASDNLVEKFEEYYGGTASDAIKQYFSASIGESYYWNDCRQQYYDLCRE |
| sequence - | LGVEVSDLTHDLEILCREKCLAVATESNQNNSIISVLEGTGEKEDRSVKLRITKKILEAISNLKEIP |
| SEQ ID NO: | KNVAPIQEIILNVAKATKETFRQVYAGNLGAPSTLEKFIAKDGQKEFDLKKLQTDLKKVIRGKSKER |
| 338 | DWCCQEELRSYVEQNTIQYDLWAWGEMENKAHTALKIKSTRNYNFAKQRLEQFKEIQSLNNLLVVKK |
| | INDFEDSEFFSGEETYTICVHHLGGKDLSKLYKAWEDDPADPENAIVVLCDDLKNNEKKEPIRNILR |
| | YIFTIRQECSAQDILAAAKYNQQLDRYKSQKANPSVLGNQGFTWTKAVILPEKAQRNDRPNSLDLRI |
| | WLYLKLRHPDGRWKKHHIPFYDTRFFCEIYAAGNSPVDICQFRTPREGYHLPKLIDQTAIRVNKKHV |
| | KAAKTEARIRLAIQQGTLPVSNLKITEISATINSKGQVRIPVKEDVGRQKGTLQIGDRECGYDQNQT |
| | ASHAYSLWEVVKEGQYHKELGCFVRFISSGDIVSITENRGNQFDQLSYEGLAYPQYADWRKKASKFV |
| | SLWQITKKNKKKEIVTVEAKEKFDAICKYQPRLYKENKEYAYLLRDIVRGKSLVELQQIRQEIFRFI |
| | EQDCGVTRLGSLSLSTLETVKAVKGIIYSYFSTALNASKNNPISDEQRKEFDPELFALLEKLELIRT |
| | RKKKQKVERIANSLIQTCLENNIKFIRGEGDLSTTNNATKKKANSRSMDWLARGVENKIRQLAPMHN |
| | ITLFGCGSLYTSHQDPLVHRNPDKAMKCRWAAIPVKDIGDWVLRKLSQNLRAKNIGTGEYYHQGVKE |
| | FLSHYELQDLEEELLKWRSDRKSNIPCWVLQNRLAEKLGNKEAVVYIPVRGGRIYFATHKVATGAVS |
| | IVFDQKQVWVCNADHVAAANIALTVKGIGEQSSDEENPDGSRIKLQLTS |
| TRAC - SEQ | AACCTCCCATTCTGCTAATGCCCAGCCTAAGTTGGGGAGACCACTCCAGATTCCAAGATGTACAGTT |
| ID NO: 339 | TGCTTTGCTGGGCCTTTTTCCCATGCCTGCCTTTACTCTGCCAGAGTTATATTGCTGGGGTTTTGAA |
| | GAAGATCCTATTAAATAAAAGAATAAGCAGTATTATTAAGTAGCCCTGCATTTCAGGTTTCCTTGAG |
| | TGCTTTGCTGGGCCTTTTTCCCATGCCTGCCTTTACTCTGCCAGAGTTATATTGCTGGGGTTTTGAA |
| | TGGCAGGCCAGGCCTGGCCGTGAACGTTCACTGAAATCATGGCCTCTTGGCCAAGATTGATAGCTTG |
| | TGCCTGTCCCTGAGTCCCAGTCCATCACGAGCAGCTGGTTTCTAAGATGCTATTTCCCGTATAAAGC |
| | ATGAGACCGTGACTTGCCAGCCCCACAGAGCCCCGCCCTTGTCCATCACTGGCATCTGGACTCCAGC |
| | CTGGGTTGGGGCAAAGAGGGAAATGAGATCATGTCCTAACCCTGATCCTCTTGTCCCACAGATATCC |
| | AGAACCCTGACCCTGCCGTGTACCAGCTGAGAGACTCTAAATCCAGTGACAAGTCTGTCTGCCTATT |
| | CACCGATTTTGATTCTCAAACAAATGTGTCACAAAGTAAGGATTCTGATGTGTATATCACAGACAAA |
| | ACTGTGCTAGACATGAGGTCTATGGACTTCAAGAGCAACAGTGCTGTGGCCTGGAGCAACAAATCTG |
| | ACTTTGCATGTGCAAACGCCTTCAACAACAGCATTATTCCAGAAGACACCTTCTTCCCCAGCCCAGG |
| | TAAGGGCAGCTTTGGTGCCTTCGCAGGCTGTTTCCTTGCTTCAGGAATGGCCAGGTTCTGCCCAGAG |
| | CTCTGGTCAATGATGTCTAAAACTCCTCTGATTGGTGGTCTCGGCCTTATCCATTGCCACCAAAACC |
| | CTCTTTTTACTAAGAAACAGTGAGCCTTGTTCTGGCAGTCCAGAGAATGACACGGGAAAAAAGCAGA |
| | TGAAGAGAAGGTGGCAGGAGAGGGCACGTGGCCCAGCCTCAGTCTCTCCAACTGAGTTCCTGCCTGC |
| | CTGCCTTTGCTCAGACTGTTTGCCCCTTACTGCTCTTCTAGGCCTCATTCTAAGCCCCTTCTCCAAG |
| | TTGCCTCTCCTTATTTCTCCCTGTCTGCCAAAAAATCTTTCCCAGCTCACTAAGTCAGTCTCACGCA |
| | GTCACTCATTAACCCACCAATCACTGATTGTGCCGGCACATGAATGCACCAGGTGTTGAAGTGGAGG |
| | AATTAAAAAGTCAGATGAGGGGTGTGCCCAGAGGAAGCACCATTCTAGTTGGGGGAGCCCATCTGTC |
| | AGCTGGGAAAAGTCCAAATAACTTCAGATTGGAATGTGTTTTAACTCAGGGTTGAGAAAACAGCTAC |
| | CTTCAGGACAAAAGTCAGGGAAGGGCTCTCTGAAGAAATGCTACTTGAAGATACCAGCCCTACCAAG |
| | GGCAGGGAGAGGACCCTATAGAGGCCTGGGACAGGAGCTCAATGAGAAAGGAGAAGAGCAGCAGGCA |
| | TGAGTTGAATGAAGGAGGCAGGGCCGGGTCACAGGGCCTTCTAGGCCATGAGAGGGTAGACAGTATT |
| | CTAAGGACGCCAGAAAGCTGTTGATCGGCTTCAAGCAGGGGAGGGACACCTAATTTGCTTTTCTTTT |
| | TTTTTTTTTTTTTTTTTTTTTTTTTTGAGATGGAGTTTTGCTCTTGTTGCCCAGGCTGGAGTGCAAT |
| | GGTGCATCTTGGCTCACTGCAACCTCCGCCTCCCAGGTTCAAGTGATTCTCCTGCCTCAGCCTCCCG |
| | AGTAGCTGAGATTACAGGCACCCGCCACCATGCCTGGCTAATTTTTTGTATTTTTAGTAGAGACAGG |
| | GTTTCACTATGTTGGCCAGGCTGGTCTCGAACTCCTGACCTCAGGTGATCCACCCGCTTCAGCCTCC |
| | CAAAGTGCTGGGATTACAGGCGTGAGCCACCACACCCGGCCTGCTTTTCTTAAAGATCAATCTGAGT |
| | GCTGTACGGAGAGTGGGTTGTAAGCCAAGAGTAGAAGCAGAAAGGGAGCAGTTGCAGCAGAGAGATG |
| | ATGGAGGCCTGGGCAGGGTGGTGGCAGGGAGGTAACCAACACCATTCAGGTTTCAAAGGTAGAACCA |
| | TGCAGGGATGAGAAAGCAAAGAGGGGATCAAGGAAGGCAGCTGGATTTTGGCCTGAGCAGCTGAGTC |
| | AATGATAGTGCCGTTTACTAAGAAGAAACCAAGGAAAAAATTTGGGGTGCAGGGATCAAAACTTTTT |
| | GGAACATATGAAAGTACGTGTTTATACTCTTTATGGCCCTTGTCACTATGTATGCCTCGCTGCCTCC |
| | ATTGGACTCTAGAATGAAGCCAGGCAAGAGCAGGGTCTATGTGTGATGGCACATGTGGCCAGGGTCA |
| | TGCAACATGTACTTTGTACAAACAGTGTATATTGAGTAAATAGAAATGGTGTCCAGGAGCCGAGGTA |
| | TCGGTCCTGCCAGGGCCAGGGGCTCTCCCTAGCAGGTGCTCATATGCTGTAAGTTCCCTCCAGATCT |
| | CTCCACAAGGAGGCATGGAAAGGCTGTAGTTGTTCACCTGCCCAAGAACTAGGAGGTCTGGGGTGGG |
| | AGAGTCAGCCTGCTCTGGATGCTGAAAGAATGTCTGTTTTTCCTTTTAGAAAGTTCCTGTGATGTCA |
| | AGCTGGTCGAGAAAAGCTTTGAAACAGGTAAGACAGGGGTCTAGCCTGGGTTTGCACAGGATTGCGG |
| | AAGTGATGAACCCGCAATAACCCTGCCTGGATGAGGGAGTGGGAAGAAATTAGTAGATGTGGGAATG |
| | AATGATGAGGAATGGAAACAGCGGTTCAAGACCTGCCCAGAGCTGGGTGGGGTCTCTCCTGAATCCC |
| | TCTCACCATCTCTGACTTTCCATTCTAAGCACTTTGAGGATGAGTTTCTAGCTTCAATAGACCAAGG |
| | ACTCTCTCCTAGGCCTCTGTATTCCTTTCAACAGCTCCACTGTCAAGAGAGCCAGAGAGAGCTTCTG |
| | GGTGGCCCAGCTGTGAAATTTCTGAGTCCCTTAGGGATAGCCCTAAACGAACCAGATCATCCTGAGG |
| | ACAGCCAAGAGGTTTTGCCTTCTTTCAAGACAAGCAACAGTACTCACATAGGCTGTGGGCAATGGTC |
| | CTGTCTCTCAAGAATCCCCTGCCACTCCTCACACCCACCCTGGGCCCATATTCATTTCCATTTGAGT |
| | TGTTCTTATTGAGTCATCCTTCCTGTGGTAGCGGAACTCACTAAGGGGCCCATCTGGACCCGAGGTA |
| | TTGTGATGATAAATTCTGAGCACCTACCCCATCCCCAGAAGGGCTCAGAAATAAAATAAGAGCCAAG |
| | TCTAGTCGGTGTTTCCTGTCTTGAAACACAATACTGTTGGCCCTGGAAGAATGCACAGAATCTGTTT |
| | GTAAGGGGATATGCACAGAAGCTGCAAGGGACAGGAGGTGCAGGAGCTGCAGGCCTCCCCCACCCAG |
| | CCTGCTCTGCCTTGGGGAAAACCGTGGGTGTGTCCTGCAGGCCATGCAGGCCTGGGACATGCAAGCC |
| | CATAACCGCTGTGGCCTCTTGGTTTTACAGATACGAACCTAAACTTTCAAAACCTGTCAGTGATTGG |
| | GTTCCGAATCCTCCTCCTGAAAGTGGCCGGGTTTAATCTGCTCATGACGCTGCGGCTGTGGTCCAGC |
| | TGAGGTGAGGGGCCTTGAAGCTGGGAGTGGGGTTTAGGGACGCGGGTCTCTGGGTGCATCCTAAGCT |
| | CTGAGAGCAAACCTCCCTGCAGGGTCTTGCTTTTAAGTCCAAAGCCTGAGCCCACCAAACTCTCCTA |
| | CTTCTTCCTGTTACAAATTCCTCTTGTGCAATAATAATGGCCTGAAACGCTGTAAAATATCCTCATT |
| | TCAGCCGCCTCAGTTGCACTTCTCCCCTATGAGGTAGGAAGAACAGTTGTTTAGAAACGAAGAAACT |
| | GAGGCCCCACAGCTAATGAGTGGAGGAAGAGAGACACTTGTGTACACCACATGCCTTGTGTTGTACT |
| | TCTCTCACCGTGTAACCTCCTCATGTCCTCTCTCCCCAGTACGGCTCTCTTAGCTCAGTAGAAAGAA |
| | GACATTACACTCATATTACACCCCAATCCTGGCTAGAGTCTCCGCACCCTCCTCCCCCAGGGTCCCC |
| | AGTCGTCTTGCTGACAACTGCATCCTGTTCCATCACCATCAAAAAAAAACTCCAGGCTGGGTGCGGG |
| | CCTGGGCAACACAGGGAGACCCCGCCTCTACAAAAAGTGAAAAAATTAACCAGGTGTGGTGCTGCAC |
| | GGCTCACACCTGTAATCCCAGCACTTTGGGAGGCAGAGGCAGGAGGAGCACAGGAGCTGGAGACCAG |
| | CCTGGGCAACACAGGGAGACCCCGCCTCTACAAAAAGTGAAAAAATTAACCAGGTGTGGTGCTGCAC |
| | ACCTGTAGTCCCAGCTACTTAAGAGGCTGAGATGGGAGGATCGCTTGAGCCCTGGAATGTTGAGGCT |
| | ACAATGAGCTGTGATTGCGTCACTGCACTCCAGCCTGGAAGACAAAGCAAGATCCTGTCTCAAATAA |
| | TAAAAAAAATAAGAACTCCAGGGTACATTTGCTCCTAGAACTCTACCACATAGCCCCAAACAGAGCC |
| | ATCACCATCACATCCCTAACAGTCCTGGGTCTTCCTCAGTGTCCAGCCTGACTTCTGTTCTTCCTCA |
| | TTCCAGATCTGCAAGATTGTAAGACAGCCTGTGCTCCCTCGCTCCTTCCTCTGCATTGCCCCTCTTC |
| | TCCCTCTCCAAACAGAGGGAACTCTCCTACCCCCAAGGAGGTGAAAGCTGCTACCACCTCTGTGCCC |
| | CCCCGGCAATGCCACCAACTGGATCCTACCCGAATTTATGATTAAGATTGCTGAAGAGCTGCCAAAC |
| | ACTGCTGCCACCCCCTCTGTTCCCTTATTGCTGCTTGTCACTGCCTGACATTCACGGCAGAGGCAAG |
| | GCTGCTGCAGCCTCCCCTGGCTGTGCACATTCCCTCCTGCTCCCCAGAGACTGCCTCCGCCATCCCA |
| | CAGATGATGGATCTTCAGTGGGTTCTCTTGGGCTCTAGGTCCTGCAGAATGTTGTGAGGGGTTTATT |
| | TTTTTTTAATAGTGTTCATAAAGAAATACATAGTATTCTTCTTCTCAAGACGTGGGGGGAAATTATC |
| | TCATTATCGAGGCCCTGCTATGCTGTGTATCTGGGCGTGTTGTATGTCCTGCTGCCGATGCCTTCAT |
| | TAAAATGATTTGGAAGAGCAGA |
| TRAC - | CAAAGAGGGAAATGAGATCATGTCCTAACCCTGATCCTCTTGTCCCACAGATATCCAGAACCCTGAC |
| Exon 1 - | CCTGCCGTGTACCAGCTGAGAGACTCTAAATCCAGTGACAAGTCTGTCTGCCTATTCACCGATTTTG |
| SEQ ID NO: | ATTCTCAAACAAATGTGTCACAAAGTAAGGATTCTGATGTGTATATCACAGACAAAACTGTGCTAGA |
| 340 | CATGAGGTCTATGGACTTCAAGAGCAACAGTGCTGTGGCCTGGAGCAACAAATCTGACTTTGCATGT |
| | GCAAACGCCTTCAACAACAGCATTATTCCAGAAGACACCTTCTTCCCCAGCCCAGGTAAGGGCAGCT |
| | TTGGTGCCTTCGCAGGCTGTTTCCTTGCTTCAGGAATG |
| TRAC - | GAGAGTCAGCCTGCTCTGGATGCTGAAAGAATGTCTGTTTTTCCTTTTAGAAAGTTCCTGTGATGTC |
| Exon 2 - | AAGCTGGTCGAGAAAAGCTTTGAAACAGGTAAGACAGGGGTCTAGCCTGGGTTTGCACAGGATTGCG |
| SEQ ID NO: | GAAGTGATGAA |
| 341 | |
| TRAC - | AGGCCTGGGACATGCAAGCCCATAACCGCTGTGGCCTCTTGGTTTTACAGATACGAACCTAAACTTT |
| Exon 3 - | CAAAACCTGTCAGTGATTGGGTTCCGAATCCTCCTCCTGAAAGTGGCCGGGTTTAATCTGCTCATGA |
| SEQ ID NO: | CGCTGCGGCTGTGGTCCAGCTGAGGTGAGGGGCCTTGAAGCTGGGAGTGGGGTTTAGGGACGCGGGT |
| 342 | CTCTGGG |
| TRAC - | CCTGGGTCTTCCTCAGTGTCCAGCCTGACTTCTGTTCTTCCTCATTCCAGATCTGCAAGATTGTAAG |
| Exon 4 - | ACAGCCTGTGCTCCCTCGCTCCTTCCTCTGCATTGCCCCTCTTCTCCCTCTCCAAACAGAGGGAACT |
| SEQ ID NO: | CTCCTACCCCCAAGGAGGTGAAAGCTGCTACCACCTCTGTGCCCCCCCGGCAATGCCACCAACTGGA |
| 343 | TCCTACCCGAATTTATGATTAAGATTGCTGAAGAGCTGCCAAACACTGCTGCCACCCCCTCTGTTCC |
| | CTTATTGCTGCTTGTCACTGCCTGACATTCACGGCAGAGGCAAGGCTGCTGCAGCCTCCCCTGGCTG |
| | TGCACATTCCCTCCTGCTCCCCAGAGACTGCCTCCGCCATCCCACAGATGATGGATCTTCAGTGGGT |
| | TCTCTTGGGCTCTAGGTCCTGCAGAATGTTGTGAGGGGTTTATTTTTTTTTAATAGTGTTCATAAAG |
| | AAATACATAGTATTCTTCTTCTCAAGACGTGGGGGGAAATTATCTCATTATCGAGGCCCTGCTATGC |
| | TGTGTATCTGGGCGTGTTGTATGTCCTGCTGCCGATGCCTTCATTAAAATGATTTGGAAGAGCAGA |
| SEQ ID NO: | MSSAIKSYKS VERPNERKNQ LLKSTIQCLE DGSAFFEKML QGLEGGITPE IVRESTEQEK |
| 348 | QQQDIALWCA VNWERPVSQD SLIHTIASDN LVEKFEEYYG GTASDAIKQY FSASIGESYY |
| (Variant | WNDCRQQYYD LCRELGVEVS DLTHDLEILC REKCLAVATE SNQNNSIISV LEGTGEKEDR |
| Cas12i2 of | SVKLRITKKI LEAISNLKEI PKNVAPIQEI ILNVAKATKE TFRQVYAGNL GAPSTLEKFI |
| SEQ ID NO: | AKDGQKEFDT KKIQTDIKKV TRGKSKERDW CCQFEIRSYV FQNTTQYDAW AWGEMENKAH |
| 3 of | TALKIKSTRN YNFAKQRLEQ FKEIQSLNNL LVVKKLNDFF DSEFFSGEET YTICVHHLGG |
| PCT/US2021/ | KDLSKLYKAW EDDPADPENA IVVLCDDLKN NEKKEPIRNI LRYIFTIRQE CSAQDILAAA |
| 025257) | KYNQQLDRYK SQKANPSVLG NQGFTWTNAV ILPEKAQRND RPNSLDLRIW LYLKLRHPDG |
| | RWKKHHIPFY DIREFQEIYA AGNSPVDTCQ FRIPREGYHL PKLTDQTAIR VNKKHVKAAK |
| | TEARIRLAIQ QGTLPVSNLK ITEISATINS KGQVRIPVKF RVGRQKGTLQ IGDRECGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVIVEAK EKFDAICKYQ PRLYKENKEY AYLLRDIVRG |
| | KSLVELQQIR QEIFRFIEQD CGVTRIGSLS LSTLETVKAV KGIIYSYFST ALNASKNNPI |
| | SDEQRKEFDP ELFALLEKLE LIRTRKKKQK VERIANSLIQ ICLENNIKFI RGEGDLSTIN |
| | NATKKKANSR SMDWLARGVE NKIRQLAPMH NITLFGCGSL YTSHQDPLVH RNPDKAMKCR |
| | WAAIPVKDIG RWVLRKLSQN LRAKNRGIGE YYHQGVKEFL SHYELQDLEE ELLKWRSDRK |
| | SNIPCWVLQN RLAEKLGNKE AVVYIPVRGG RIYFATHKVA IGAVSIVEDQ KQVWVCNADH |
| | VAAANIALIG KGIGEQSSDE ENPDGSRIKL QLTS |
| SEQ ID NO: | MSSAIKSYKS VLRPNERKNQ LLKSTIQCLE DGSAFFFKML QGLFGGITPE IVRFSTEQEK |
| 349 | QQQDIALWCA VNWERPVSQD SLTHTIASDN LVEKFEEYYG GTASDAIKQY FSASIGESYY |
| (Variant | WNDCRQQYYD LCRELGVEVS DLTHDLEILC REKCLAVATE SNQNNSIISV LFGIGEKEDR |
| Cas12i2 of | SVKLRITKKI LEAISNLKEI PKNVAPIQEI ILNVAKATKE TFRQVYAGNL GAPSTLEKFI |
| SEQ ID NO: | AKDGQKEFDT KKLQTDIKKV TRGKSKERDW CCQEELRSYV FQNTTQYDLW AWGEMENKAR |
| 4 of | TALKIKSTRN YNFAKQRLEQ FKEIQSLNNL LVVKKINDFF DSEFFSGEET YTICVHHLGG |
| PCT/US2021/ | KDLSKLYKAW EDDPADPENA IVVLCDDLKN NEKKEPIRNI LRYIFTIRQE CSAQDILAAA |
| 025257) | KYNQQLDRYK SQKANPSVLG NQGFTWINAV ILPEKAQRND RPNSLDLRIW LYLKLRHPDG |
| | RWKKHHIPFY DIRFFQEIYA AGNSPVDTCQ FRIPRFGYHL PKLIDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVIVEAK EKFDAICKYQ PRLYKENKEY AYLERDIVRG |
| | KSLVELQQIR QEIFREIEQD CGVTRIGSLS LSTLETVKAV KGIIYSYFST ALNASKNNPI |
| | SDEQRKEFDP ELFALLEKLE LIRTRKKKQK VERIANSLIQ ICLENNIKFI RGEGDLSTIN |
| | NATKKKANSR SMDWLARGVE NKIRQLAPMH NITLFGCGSL YTSHQDPLVH RNPDKAMKQR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA IGAVSIVEDQ KQVWVCNADH |
| | VAAANIALTG KGIGEQSSDE ENPDGSRIKL QLTS |
| SEQ ID NO: | MSSAIKSYKS VLRPNERKNQ LLKSTIQCLE DGSAFFFKML QGLEGGITPE IVRESTEQEK |
| 350 | QQQDIALWCA VNWERPVSQD SLTHTIASDN LVEKFEEYYG GTASDAIKQY FSASIGESYY |
| (Variant | WNDCRQQYYD LCRELGVEVS DLTHDLEILC REKCLAVATE SNQNNSIISV LFGTGEKEDR |
| Cas12i2 of | SVKLRITKKI LEAISNLKEI PKNVAPIQEI ILNVAKATKE TFRQVYAGNL GAPSTLEKFI |
| SEQ ID NO: | AKDGQKEFDL KKLQTDLKKV IRGKSKERDW CCQEELRSYV EQNTIQYDLW AWGEMENKAH |
| 5 of | TALKIKSTRN YNFAKQRLEQ FKEIQSLNNL LVVKKLNDFF DSEFFSGEET YTICVHHLGG |
| PCT/US2021/ | KDLSKLYKAW EDDPADPENA IVVLCDDLKN NEKKEPIRNI LRYIFTIRQE CSAQDILAAA |
| 025257) | KYNQQLDRYK SQKANPSVLG NQGFTWTNAV ILPEKAQRND RPNSLDLRIW LYLKLRHPDG |
| | RWKKHHIPFY DIRFFQEIYA AGNSPVDTCQ FRIPRFGYHL PKLTDQTAIR VNKKHVKAAK |
| | TEARIRLAIQ QGTLPVSNLK ITEISATINS KGQVRIPVKF RVGRQKGTLQ IGDRECGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVIVEAK EKFDAICKYQ PRLYKENKEY AYLERDIVRG |
| | KSLVELQQIR QEIFRFIEQD CGVTRIGSLS LSTLETVKAV KGIIYSYFST ALNASKNNPI |
| | SDEQRKEFDP ELFALLEKLE LIRTRKKKQK VERIANSLIQ ICLENNIKFI RGEGDLSTIN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA IGAVSIVEDQ KQVWVCNADH |
| | VAAANIALTG KGIGEQSSDE ENPDGGRIKL QLTS |
| SEQ ID NO: | MSSAIKSYKS VLRPNERKNQ LLKSTIQCLE DGSAFFEKML QGLEGGITPE IVRESTEQEK |
| 351 | QQQDIALWCA VNWFRPVSQD SLTHTIASDN LVEKFEEYYG GTASDAIKQY FSASIGESYY |
| (Variant | WNDCRQQYYD LCRELGVEVS DLTHDLEILC REKCLAVATE SNQNNSIISV LFGTGEKEDR |
| Cas12i2 of | SVKLRITKKI LEAISNLKEI PKNVAPIQEI ILNVAKATKE TFRQVYAGNL GAPSTLEKFI |
| SEQ ID NO: | AKDGQKEFDL KKLQTDLKKV IRGKSKERDW CCQEELRSYV EQNTIQYDLW AWGEMENKAH |
| 495 of | TALKIKSTRN YNFAKQRLEQ FKEIQSLNNL LVVKKLNDEF DSEFFSGEET YTICVHHLGG |
| PCT/US2021/ | KDLSKLYKAW EDDPADPENA IVVLCDDLKN NFKKEPIRNI LRYIFTIRQE CSAQDILAAA |
| 025257) | KYNQQLDRYK SQKANPSVLG NQGETWTNAV ILPEKAQRND RPNSLDLRIW LYLKERAPDG |
| | RWKKHHIPFY DIREFQEIYA AGNSPVDICQ FRIPREGYHL PKLIDQTAIR VNKKHVKAAK |
| | TEARIRLAIQ QGTLPVSNLK ITEISATINS KGQVRIPVKF RVGRQKGTLQ IGDRECGYDQ |
| | NQTASHAYSL WEVVKEGQYH KELRCRVRFI SSGDIVSITE NRGNQFDQLS YEGLAYPQYA |
| | DWRKKASKFV SLWQITKKNK KKEIVIVEAK EKFDAICKYQ PRLYKENKEY AYLLRDIVRG |
| | KSLVELQQIR QEIFRFIEQD CGVIRLGSLS LSTLETVKAV KGIIYSYFST ALNASKNNPI |
| | SDEQRKEEDP ELFALLEKLE LIRTRKKKQK VERIANSLIQ TCLENNIKFI RGEGDLSTIN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGIGE YYHQGVKEFL SHYELQDLEE ELLKWRSDRK |
| | SNIPCWVLQN RLAEKLGNKE AVVYIPVRGG RIYFATAKVA IGAVSIVEDQ KQVWVCNADH |
| | VAAANIALIG KGIGRQSSDE ENPDGGRIKL QLTS |
| SEQ ID NO: | MSSAIKSYKS VLRPNERKNQ LIKSTIQCLE DGSAFFEKML QGLEGGITPE IVRESTEQEK |
| 352 | QQQDIALWCA VNWERPVSQD SLTHTIASDN LVEKFEEYYG GTASDAIKQY FSASIGESYY |
| (Variant | WNDCRQQYYD LCRELGVEVS DLTHDLEILC REKCLAVATE SNQNNSIISV LFGTGEKEDR |
| Cas12i2 of | SVKIRITKKI LEAISNLKEI PKNVAPIQEI ILNVAKATKE IFRQVYAGNL GAPSTLEKFI |
| SEQ ID NO: | AKDGQKEFDL KKLQIDLKKV IRGKSKERDW CCQEELRSYV EQNTIQYDLW AWGEMENKAH |
| 496 of | TALKIKSTRN YNFAKQRLEQ FKEIQSLNNL LVVKKLNDFF DSEFFSGEET YTICVHHLGG |
| PCT/US2021/ | KDLSKLYKAW EDDPADPENA IVVLCDDLKN NEKKEPIRNI LRYIFTIRQE CSAQDILAAA |
| 025257) | KYNQQLDRYK SQKANPSVLG NQGETWINAV ILPEKAQRND RPNSLDLRIW LYLKLRHPDG |
| | RWKKHHIPFY DIRFFQEIYA AGNSPVDTCQ FRIPRFGYHL PKLIDQTAIR VNKKHVKAAK |
| | TEARIRLAIQ QGTLPVSNLK ITEISATINS KGQVRIPVKF RVGROKGTLQ IGDRECGYDO |
| | NOTASHAYSL WEVVKEGQYH KELRCRVRFI SSGDIVSITE NRGNQFDQLS YEGLAYPQYA |
| | DWRKKASKFV SLWQITKKNK KKEIVIVEAK EKFDAICKYQ PRLYKENKEY AYLLRDIVRG |
| | KSLVELQQIR QRTFRFTROD CGVTRIGSLS LSTLETVKAV KGTTYSYFST AINASKNNPT |
| | SDEQRKEFDP ELFALLEKLE LIRTRKKKOK VERIANSLIQ ICLENNIKFI RGEGDLSTIN |
| | NATKKKANSR SMDWLARGVE NKIRQLATMH NITLFGCGSL YTSHODPLVH RNPDKAMKCR |
| | WAAIPVKDIG DWVLRKLSON LRAKNRGIGE YYHQGVKEFL SHYELQDLEE ELLKWRSDRK |
| | SNIPCWVLON RLAEKLGNKE AVVYIPVRGG RIYFATHKVA IGAVSIVEDQ KOVWVCNADH |
| | VAAANIALTG KGIGROSSDE ENPDGGRIKL QLIS |
| SEQ ID NO: | ATGGCTTCCATCTCTAGGCCATACGGCACCAAGCTGCGACCGGACGCACGGAAGAAGGAGATGCTCG |
| 353 | ATAAGTTCTTTAATACACTGACTAAGGGTCAGCGCGTGTTCGCAGACCTGGCCCTGTGCATCTATGG |
| (Nucleotide | CTCCCTGACCCTGGAGATGGCCAAGTCTCTGGAGCCAGAAAGTGATTCAGAACTGGTGTGCGCTATT |
| sequence | GGGTGGTTTCGGCTGGTGGACAAGACCATCTGGTCCAAGGATGGCATCAAGCAGGAGAATCTGGTGA |
| encoding | AACAGTACGAAGCCTATTCCGGAAAGGAGGCTTCTGAAGTGGTCAAAACATACCTGAACAGCCCCAG |
| Cas12i4) | CTCCGACAAGTACGTGTGGATCGATTGCAGGCAGAAATTCCTGAGGTTTCAGCGCGAGCTCGGCACT |
| | CGCAACCTGTCCGAGGACTTCGAATGTATGCTCTTTGAACAGTACATTAGACTGACCAAGGGCGAGA |
| | TCGAAGGGTATGCCGCTATTTCAAATATGTTCGGAAACGGCGAGAAGGAAGACCGGAGCAAGAAAAG |
| | AATGTACGCTACACGGATGAAAGATTGGCTGGAGGCAAACGAAAATATCACTTGGGAGCAGTATAGA |
| | GAGGCCCTGAAGAACCAGCTGAATGCTAAAAACCTGGAGCAGGTTGTGGCCAATTACAAGGGGAACG |
| | CTGGCGGGGCAGACCCCTTCTTTAAGTATAGCTTCTCCAAAGAGGGAATGGTGAGCAAGAAAGAACA |
| | TGCACAGCAGCTCGACAAGTTCAAAACCGTCCTGAAGAACZAAGCCCGGGACCTGAATTTTCCAAAC |
| | AAGGAGAAGCTGAAGCAGTACCTGGAGGCCGAAATCGGCATTCCGGTCGACGCTAACGTGTACTCCC |
| | AGATGTTCTCTAACGGGGTGAGTGAGGTCCAGCCTAAGACCACACGGAATATGTCTTTTAGTAACGA |
| | GAAACTGGATCTGCTCACTGAACTGAAGGACCTGAACAAGGGCGATGGGTTCGAGTACGCCAGAGAA |
| | GTGCTGAACGGGTTCTTTGACTCCGAGCTCCACACTACCGAGGATAAGTTTAATATCACCTCTAGGT |
| | ACCTGGGAGGCGACAAATCAAACCGCCTGAGCAAACTCTATAAGATCTGGAAGAAAGAGGGTGTGGA |
| | CTGCGAGGAAGGCATTCAGCAGTTCTGTGAAGCCGTCAAAGATAAGATGGGCCAGATCCCCATTCGA |
| | AATGTGCTGAAGTACCTGTGGCAGTTCCGGGAGACAGTCAGTGCCGAGGATTTTGAAGCAGCCGCTA |
| | AGGCTAACCATCTGGAGGAAAAGATCAGCCGGGTGAAAGCCCACCCAATCGTGATTAGCAATAGGTA |
| | CTGGGCTTTTGGGACTTCCGCACTGGTGGGAAACATTATGCCCGCAGACAAGAGGCATCAGGGAGAG |
| | TATGCCGGTCAGAATTTCAAAATGTGGCTGGAGGCTGAACTGCACTACGATGGCAAGAAAGCAAAGC |
| | ACCATCTGCCTTTTTATAACGCCCGCTTCTTTGAGGAAGTGTACTGCTATCACCCCTCTGTCGCCGA |
| | GATCACTCCTTTCAAAACCAAGCAGTTTGGCTGTGAAATCGGGAAGGACATTCCAGATTACGTGAGC |
| | GTCGCTCTGAAGGACAATCCGTATAAGAAAGCAACCAAACGAATCCTGCGTGCAATCTACAATCCCG |
| | TCGCCAACACAACTGGCGTTGATAAGACCACAAACTGCAGCTTCATGATCAAACGCGAGAATGACGA |
| | ATATAAGCTGGTCATCAACCGAAAAATTTCCGTGGATCGGCCTAAGAGAATCGAAGTGGGCAGGACA |
| | ATTATGGGGTACGACCGCAATCAGACAGCTAGCGATACTTATTGGATTGGCCGGCTGGTGCCACCTG |
| | GAACCCGGGGCGCATACCGCATCGGAGAGTGGAGCGTCCAGTATATTAAGTCCGGGCCTGTCCTGTC |
| | TAGTACTCAGGGAGTTAACAATTCCACTACCGACCAGCTGGTGTACAACGGCATGCCATCAAGCTCC |
| | GAGCGGTTCAAGGCCTGGAAGAAAGCCAGAATGGCTTTTATCCGAAAACTCATTCGTCAGCTGAATG |
| | ACGAGGGACTGGAATCTAAGGGTCAGGATTATATCCCCGAGAACCCTTCTAGTTTCGATGTGCGGGG |
| | CGAAACCCTGTACGTCTTTAACAGTAATTATCTGAAGGCCCTGGTGAGCAAACACAGAAAGGCCAAG |
| | AAACCTGTTGAGGGGATCCTGGACGAGATTGAAGCCTGGACATCTAAAGACAAGGATTCATGCAGCC |
| | TGATGCGGCTGAGCAGCCTGAGCGATGCTTCCATGCAGGGAATCGCCAGCCTGAAGAGTCTGATTAA |
| | CAGCTACTTCAACAAGAATGGCTGTAAAACCATCGAGGACAAAGAAAAGTTTAATCCCGTGOTGTAT |
| | GCCAAGCTGGTTGAGGTGGAACAGCGGAGAACAAACAAGCGGTCTGAGAAAGTGGGAAGAATCGCAG |
| | GTAGTCTGGAGCAGCTGGCCCTGCTGAACGGGGTTGAGGTGGTCATCGGCGAAGCTGACCTGGGGGA |
| | GGTCGAAAAAGGAAAGAGTAAGAAACAGAATTCACGGAACATGGATTGGTGCGCAAAGCAGGTGGCA |
| | CAGCGGCTGGAGTACAAACTGGCCTTCCATGGAATCGGTTACTTTGGAGTGAACCCCATGTATACCA |
| | GCCACCAGGACCCTTTCGAACATAGGCGCGTGGCTGATCACATCGTCATGCGAGCACGTTTTGAGGA |
| | AGTCAACGTGGAGAACATTGCCGAATGGCACGTGCGAAATTTCTCAAACTACCTGCGTGCAGACAGC |
| | GGCACTGGGCTGTACTATAAGCAGGCCACCATGGACTTCCTGAAACATTACGGTCTGGAGGAACACG |
| | CTGAGGGCCTGGAAAATAAGAAAATCAAGTTCTATGACTTTAGAAAGATCCTGGAGGATAAAAACCT |
| | GACAAGCGTGATCATTCCAAAGAGGGGGGGGCGCATCTACATGGCCACCAACCCAGTGACATCCGAC |
| | TCTACCCCGATTACATACGCCGGCAAGACTTATAATAGGTGTAACGCTGATGAGGTGGCAGCCGCTA |
| | ATATCGTTATTTCTGTGCTGGCTCCCCGCAGTAAGAAAAACGAGGAACAGGACGATATCCCTCTGAT |
| | TACCAAGAAAGCCGAGAGTAAGTCACCACCGAAAGACCGGAAGAGATCAAAAACAAGCCAGCTGCCT |
| | CAGAAA |
| SEQ ID NO: | MASISRPYGTKLRPDARKKEMLDKFFNILTKGQRVFADLALCIYGSLTLEMAKSLEPESDSELVCAI |
| 354 | GWFRLVDKTIWSKDGIKQENLVKQYEAYSGKEASEVVKTYLNSPSSDKYVWIDCRQKFLRFQRELGT |
| Cas12i4 | RNLSEDFECMLFEQYIRLTKGEIEGYAAISNMFGNGEKEDRSKKRMYAIRMKDWLEANENITWEQYR |
| amino acid | EALKNQLNAKNLEQVVANYKGNAGGADPFFKYSFSKEGMVSKKEHAQQLDKFKTVLKNKARDLNFPN |
| sequence of | KEKLKQYLEAETGIPVDANVYSQMFSNGVSEVQPKTTRNMSFSNEKIDLLTELKDINKGQGFEYARE |
| SEQ ID NO: | VLNGFFDSELHTTEDKENITSRYLGGDKSNRLSKLYKIWKKEGVDCEEGIQQFCEAVKDKMGQIPIR |
| 14 of U.S. | NVLKYLWQFRETVSAEDFEAAAKANHLEEKISRVKAHPIVISNRYWAFGISALVGNIMPADKRHQGE |
| Patent No | YAGQNEKMWLEAELHYDGKKAKHHLPFYNARFFEEVYCYHPSVAEIIPFKTKQFGCEIGKDIPDYVS |
| 10,808,245) | VALKDNPYKKATKRILRAIYNPVANTTGVDKTINCSEMIKRENDEYKLVINRKISVDRPKRIEVGRI |
| | IMGYDRNQTASDTYWIGRLVPPGTRGAYRIGEWSVQYIKSGPVLSSTQGVNNSTIDQLVYNGMPSSS |
| | ERFKAWKKARMAFIRKLIRQLNDEGLESKGQDYIPENPSSFDVRGETLYVENSNYLKALVSKARKAK |
| | KPVEGILDEIEAWTSKDKDSCSLMRLSSLSDASMQGIASLKSLINSYENKNGCKTIEDKEKENPVLY |
| | AKLVEVEQRRINKRSEKVGRIAGSLEQLALLNGVEVVIGEADLGEVEKGKSKKQNSRNMDWCAKQVA |
| | QRLEYKLAFHGIGYFGVNPMYTSHQDPFEHRRVADHIVMRARFEEVNVENIAEWHVRNESNYLRADS |
| | GTGLYYKQATMDELKHYGLEEHAEGLENKKIKFYDERKILEDKNLTSVIIPKRGGRIYMATNPVTSD |
| | STPITYAGKTYNRCNADEVAAANIVISVLAPRSKKNEEQDDIPLITKKAESKSPPKDRKRSKTSQLP |
| | QK |
| SEQ ID NO: | MASISRPYGT KLRPDARKKE MLDKFENTLI KGQRVFADLA LCIYGSLILE MAKSLEPESD |
| 355 | SELVCAIGWF RLVDKTIWSK DGIKQENLVK QYEAYSGKEA SEVVKTYLNS PSSDKYVWID |
| (Variant | CRQKFLRFQR ELGIRNLSED FECMLFEQYI RLIKGEIEGY AAISNMEGNG EKEDRSKKRM |
| Cas12i4) | YATRMKDWLE ANENITWEQY REALKNQLNA KNLEQVVANY KGNAGGADPF FKYSFSKEGM |
| | VSKKEHAQQL DKFKTVLKNK ARDLNEPNKE KLKQYLEZEI GIPVDANVYS QMFSNGVSEV |
| | QPKTTRNMSF SNEKLDLLTE LKDLNKGDGE EYAREVINGE FDSELHTTED KENITSRYLG |
| | GDKSNRLSKL YKIWKKEGVD CEEGIQQFCE AVKDKMGQIP IRNVLKYLWQ FRETVSAEDE |
| | EAAAKANHLE EKISRVKARP IVISNRYWAF GTSALVGNIM PADKRHQGEY AGQNFKMWLE |
| | AELHYDGKKA KHALPFYNAR FFEEVYCYHP SVAEITPEKI KQFGCEIGKD IPDYVSVALK |
| | DNPYKKATKR ILRAIYNPVA NTTGVDKTIN CSFMIKREND EYKLVINRKI SRDRPKRIEV |
| | GRTIMGYDRN QTASDTYWIG RLVPPGTRGA YRIGEWSVQY IKSGPVISST QGVNNSTIDQ |
| | LVYNGMPSSS ERFKAWKKAR MAFIRKLIRQ LNDEGLESKG QDYIPENPSS FDVRGEILYV |
| | ENSNYLKALV SKARKAKKPV EGILDEIEAW TSKDKDSCSL MRLSSLSDAS MQGIASLKSL |
| | INSYFNKNGC KTIEDKEKEN PVLYAKLVEV EQRRINKRSE KVGRIAGSLE QLALLNGVEV |
| | VIGEADLGEV EKGKSKKQNS RNMDWCAKQV AQRLEYKLAF AGIGYFGVNP MYTSHQDPFE |
| | HRRVADHIVM RARFEEVNVE NIAEWHVRNE SNYLRADSGT GLYYKQAIMD FLKHYGLEEH |
| | AEGLENKKIK FYDERKILED KNLISVIIPK RGGRIYMATN PVTSDSTPIT YAGKTYNRCN |
| | ADEVAAANIV ISVLAPRSKK NREQDDIPLI TKKAESKSPP KDRKRSKISQ LPQK |
| SEQ ID NO: | MASISRPYGT KLRPDARKKE MLDKFENTLI KGQRVFADLA LCIYGSLILE MAKSLEPESD |
| 356 | SELVCAIGWF RLVDKTIWSK DGIKQENLVK QYEAYSGKEA SEVVKTYLNS PSSDKYVWID |
| (Variant | CRQKFLRFQR ELGIRNLSED FECMLFEQYI RLTKGEIEGY AAISNMEGNG EKEDRSKKRM |
| Cas12i4) | YATRMKDWLE ANENITWEQY REALKNQLNA KNLEQVVANY KGNAGGADPF FKYSFSKEGM |
| | VSKKEHAQQL DKFKTVLKNK ARDLNEPNKE KLKQYLEAEI GIPVDANVYS QMFSNGVSEV |
| | QPKTTRNMSF SNEKLDLLTE LKDLNKGDGE EYAREVLNGF FDSELHTTED KENITSRYLG |
| | GDKSNRLSKL YKIWKKEGVD CEEGIQQFCE AVKDKMGQIP IRNVLKYLWQ FRETVSAEDE |
| | EAAAKANHLE EKISRVKAHP IVISNRYWAF GTSALVGNIM PADKRHQGEY AGQNFKMWLR |
| | AELHYDGKKA KRALPFYNAR FFEEVYCYHP SVAEITPEKT KQFGCEIGKD IPDYVSVALK |
| | DNPYKKATKR ILRAIYNPVA NTTRVDKIIN CSEMIKREND EYKLVINRKI SRDRPKRIEV |
| | GRTIMGYDRN QTASDTYWIG RLVPPGTRGA YRIGEWSVQY IKSGPVLSST QGVNNSTIDQ |
| | LVYNGMPSSS ERFKAWKKAR MAFIRKLIRQ LNDEGLESKG QDYIPENPSS EDVRGETLYV |
| | ENSNYLKALV SKARKAKKPV EGILDEIEAW TSKDKDSCSL MRLSSLSDAS MQGIASLKSL |
| | INSYFNKNGC KTIEDKEKEN PVLYAKLVEV EQRRINKRSE KVGRIAGSLE QLALLNGVEV |
| | VIGEADIGEV EKGKSKKQNS RNMDWCAKQV AQRLEYKLAF HGIGYFGVNP MYTSHQDPFE |
| | HRRVADHIVM RARFEEVNVE NIAEWHVRNE SNYLRADSGT GLYYKQAIMD FLKHYGLEER |
| | AEGLENKKIK FYDERKILED KNLISVIIPK RGGRIYMATN PVTSDSTPIT YAGKTYNRCN |
| | ADEVAAANIV ISVLAPRSKK NREQDDIPLI IKKAESKSPP KDRKRSKISQ LPQK |
| SEQ ID NO: | MSNKEKNASETRKAYTTKMIPRSHDRMKLLGNFMDYLMDGTPIFFELWNQFGGGIDRDIISGTANKD |
| 357 | KISDDLLLAVNWFKVMPINSKPQGVSPSNLANLFQQYSGSEPDIQAQEYFASNFDTEKHQWKDMRVE |
| (Cas12i1 of | YERLLAELQLSRSDMHHDLKLMYKEKCIGLSLSTAHYITSVMFGIGAKNNRQTKHQFYSKVIQLLEE |
| SEQ ID NO: | STQINSVEQLASIILKAGDCDSYRKLRIRCSRKGATPSILKIVQDYELGINHDDEVNVPSLIANLKE |
| 3 of U.S. | KLGRFEYECEWKCMEKTKAFLASKVGPYYLGSYSAMLENALSPIKGMTTKNCKFVIKQTDAKNDTKY |
| Pat. No. | ENEPFGKIVEGFFDSPYFESDINVKWVLHPHHIGESNIKILWEDLNAIHSKYEEDIASLSEDKKEKR |
| 10,808,245) | IKVYQGDVCQTINTYCEEVGKEAKTPLVQLLRYLYSRKDDIAVDKIIDGITFLSKKHKVEKQKINPV |
| | IQKYPSFNFGNNSKLIGKIISPKDKLKHNLKCNRNQVDNYIWIEIKVLNTKIMRWEKHHYALSSTRE |
| | LEEVYYPATSENPPDALAARFRIKINGYEGKPALSARQIEQIRSAPVGLRKVKKRQMRLEAARQQNL |
| | LPRYTWGKDFNINICKRENNFEVTLATKVKKKKEKNYKVVLGYDANIVRKNTYAAIEAHANGDGVID |
| | YNDLPVKPIESGFVIVESQVRDKSYDQLSYNGVKLLYCKPHVESRRSFLEKYRNGTMKDNRGNNIQI |
| | DFMKDFEAIADDETSLYYFNMKYCKLLQSSIRNHSSQAKEYREEIFELLRDGKLSVLKLSSLSNLSE |
| | VMFKVAKSLIGTYFGHLLKKPKNSKSDVKAPPITDEDKQKADPEMFALRLALEEKRLNKVKSKKEVI |
| | ANKIVAKALELRDKYGPVLIKGENISDITKKGKKSSINSFLMDWLARGVANKVKEMVMMHQGLEFVE |
| | VNPNFISHQDPFVHKNPENTFRARYSRCTPSELTEKNRKEILSFLSDKPSKRPTNAYYNEGAMAFLA |
| | TYGLKKNDVLGVSLEKFKQIMANILHQRSEDQLLFPSRGGMFYLATYKLDADATSVNWNGKQFWVCN |
| | ADLVAAYNVGLVDIQKDEKKK |
| SEQ ID NO: | MSISNNNILPYNPKLLPDDRKHKMLVDIFNQLDLIRNNLHDMIIALYGALKYDNIKQFASKEKPHIS |
| 358 | ADALCSINWERLVKINERKPAIESNQIISKFIQYSGHTPDKYALSHITGNHEPSHKWIDCREYAINY |
| (Cas12i3 of | ARIMHLSFSQFQDLATACLNCKILILNGTLISSWAWGANSALFGGSDKENFSVKAKILNSFIENLKD |
| SEQ ID NO: | EMNITKFQVVEKVCQQIGSSDAADLFDLYRSTVKDGNRGPATGRNPKVMNLFSQDGEISSEQREDFI |
| 14 of U.S. | ESFQKVMQEKNSKQIIPELDKLKYHLVKQSGLYDIYSWAAAIKNANSTIVASNSSNLNTILNKTEKQ |
| Pat. No. | QTFEELRKDEKIVACSKILLSVNDTLPEDLHYNPSTSNLGKNLDVFFDLLNENSVATIENKEEKNKI |
| 10,808,245) | VKECVNQYMEECKGLNKPPMPVLLTFISDYAHKHQAQDFLSAAKMNFIDLKIKSIKVVPTVHGSSPY |
| | TWISNLSKKNKDGKMIRIPNSSLIGWIIPPEEIHQQKFAGQNPIIWAVLRVYCNNKWEMHHFPFSDS |
| | RFFTEVYAYKPNLPYLPGGENRSKRFGYRHSINLSNESRQILLDKSKYAKANKSVLRCMENMTHNVV |
| | EDPKTSLNIRIKTDKNNSPVLDDKGRIIFVMQINHRILEKYNNTKIEIGDRILAYDQNQSENHTYAI |
| | LQRTEEGSHAHQENGWYVRVLETGKVTSIVQGLSGPIDQLNYDGMPVTSHKENCWQADRSAFVSQFA |
| | SIKISETEIFDEAYQAINAQGAYTWNLFYLRILRKALRVCHMENINQFREEILAISKNRLSPMSLGS |
| | LSQNSLKMIRAFKSIINCYMSRMSFVDELQKKEGDLELHTIMRLIDNKLNDKRVEKINRASSFLINK |
| | AHSMGCKMIVGESDLPVADSKTSKKQNVDRMDWCARALSHKVEYACKLMGLAYRGIPAYMSSHQDPL |
| | VALVESKRSVERPREVVADKSDVKQHHLDNLRRMLNSKTKVGTAVYYREAVELMCEELGIHKTDMAK |
| | GKVSLSDFVDKFIGEKAIFPQRGGRFYMSTKRLTTGAKLICYSGSDVWLSDADEIAAINIGMEVVCD |
| | QTGAFKKKKKEKLDDEECDILPERPM |

What is claimed is:

1. A composition comprising an RNA guide, wherein the RNA guide comprises (i) a spacer sequence that is substantially complementary to a target sequence within a TRAC gene and (ii) a direct repeat sequence; wherein the target sequence is adjacent to a protospacer adjacent motif (PAM) comprising the sequence 5′-NTTN-3′.

2. The composition of claim 1, wherein the target sequence is within exon 1, exon 2, exon 3, or exon 4 of the TRAC gene.

3. The composition of claim 1 or 2, wherein the TRAC gene comprises the sequence of SEQ ID NO: 339, the reverse complement of SEQ ID NO: 339, a variant of the sequence of SEQ ID NO: 339, or the reverse complement of a variant of SEQ ID NO: 339.

4. The composition of any one of claims 1 to 3, wherein the spacer sequence comprises:
a. nucleotide 1 through nucleotide 16 of a sequence that is at least 90% identical to a sequence of any one of SEQ ID NOs: 174-336;
b. nucleotide 1 through nucleotide 17 of a sequence that is at least 90% identical to a sequence of any one of SEQ ID NOs: 174-336;
c. nucleotide 1 through nucleotide 18 of a sequence that is at least 90% identical to a sequence of any one of SEQ ID NOs: 174-336;
d. nucleotide 1 through nucleotide 19 of a sequence that is at least 90% identical to a sequence of any one of SEQ ID NOs: 174-336;
e. nucleotide 1 through nucleotide 20 of a sequence that is at least 90% identical to a sequence of any one of SEQ ID NOs: 174-336;
f. nucleotide 1 through nucleotide 21 of a sequence that is at least 90% identical to a sequence of any one of SEQ ID NOs: 174-336;
g. nucleotide 1 through nucleotide 22 of a sequence that is at least 90% identical to a sequence of any one of SEQ ID NOs: 174-336;
h. nucleotide 1 through nucleotide 23 of a sequence that is at least 90% identical to a sequence of any one of SEQ ID NOs: 174-336;
i. nucleotide 1 through nucleotide 24 of a sequence that is at least 90% identical to a sequence of any one of SEQ ID NOs: 174-336;
j. nucleotide 1 through nucleotide 25 of a sequence that is at least 90% identical to a sequence of any one of SEQ ID NOs: 174-275 and 277-336;
k. nucleotide 1 through nucleotide 26 of a sequence that is at least 90% identical to a sequence of any one of SEQ ID NOs: 174-275 and 277-336;
l. nucleotide 1 through nucleotide 27 of a sequence that is at least 90% identical to a sequence of any one of SEQ ID NOs: 174-275 and 277-336;
m. nucleotide 1 through nucleotide 28 of a sequence that is at least 90% identical to a sequence of any one of SEQ ID NOs: 174-275 and 277-336;
n. nucleotide 1 through nucleotide 29 of a sequence that is at least 90% identical to a sequence of any one of SEQ ID NOs: 174-275 and 277-336; or
o. nucleotide 1 through nucleotide 30 of a sequence that is at least 90% identical to a sequence of any one of SEQ ID NOs: 174-275 and 277-336.

5. The composition of any one of claims 1 to 4, wherein the spacer sequence comprises:
a. nucleotide 1 through nucleotide 16 of any one of SEQ ID NOs: 174-336;
b. nucleotide 1 through nucleotide 17 of any one of SEQ ID NOs: 174-336;
c. nucleotide 1 through nucleotide 18 of any one of SEQ ID NOs: 174-336;
d. nucleotide 1 through nucleotide 19 of any one of SEQ ID NOs: 174-336;
e. nucleotide 1 through nucleotide 20 of any one of SEQ ID NOs: 174-336;
f. nucleotide 1 through nucleotide 21 of any one of SEQ ID NOs: 174-336;
g. nucleotide 1 through nucleotide 22 of any one of SEQ ID NOs: 174-336;
h. nucleotide 1 through nucleotide 23 of any one of SEQ ID NOs: 174-336;
i. nucleotide 1 through nucleotide 24 of any one of SEQ ID NOs: 174-336;
j. nucleotide 1 through nucleotide 25 of any one of SEQ ID NOs: 174-275 and 277-336;
k. nucleotide 1 through nucleotide 26 of any one of SEQ ID NOs: 174-275 and 277-336;
l. nucleotide 1 through nucleotide 27 of any one of SEQ ID NOs: 174-275 and 277-336;
m. nucleotide 1 through nucleotide 28 of any one of SEQ ID NOs: 174-275 and 277-336;
n. nucleotide 1 through nucleotide 29 of any one of SEQ ID NOs: 174-275 and 277-336; or
o. nucleotide 1 through nucleotide 30 of any one of SEQ ID NOs: 174-275 and 277-336.

6. The composition of any one of claims 1 to 5, wherein the direct repeat comprises:
a. nucleotide 1 through nucleotide 36 of a sequence that is at least 90% identical to a sequence of any one of SEQ ID NOs: 1-8;
b. nucleotide 2 through nucleotide 36 of a sequence that is at least 90% identical to a sequence of any one of SEQ ID NOs: 1-8;
c. nucleotide 3 through nucleotide 36 of a sequence that is at least 90% identical to a sequence of any one of SEQ ID NOs: 1-8;
d. nucleotide 4 through nucleotide 36 of a sequence that is at least 90% identical to a sequence of any one of SEQ ID NOs: 1-8;
e. nucleotide 5 through nucleotide 36 of a sequence that is at least 90% identical to a sequence of any one of SEQ ID NOs: 1-8;
f. nucleotide 6 through nucleotide 36 of a sequence that is at least 90% identical to a sequence of any one of SEQ ID NOs: 1-8;
g. nucleotide 7 through nucleotide 36 of a sequence that is at least 90% identical to a sequence of any one of SEQ ID NOs: 1-8;
h. nucleotide 8 through nucleotide 36 of a sequence that is at least 90% identical to a sequence of any one of SEQ ID NOs: 1-8;
i. nucleotide 9 through nucleotide 36 of a sequence that is at least 90% identical to a sequence of any one of SEQ ID NOs: 1-8;
j. nucleotide 10 through nucleotide 36 of a sequence that is at least 90% identical to a sequence of any one of SEQ ID NOs: 1-8;
k. nucleotide 11 through nucleotide 36 of a sequence that is at least 90% identical to a sequence of any one of SEQ ID NOs: 1-8;
l. nucleotide 12 through nucleotide 36 of a sequence that is at least 90% identical to a sequence of any one of SEQ ID NOs: 1-8;
m. nucleotide 13 through nucleotide 36 of a sequence that is at least 90% identical to a sequence of any one of SEQ ID NOs: 1-8;
n. nucleotide 14 through nucleotide 36 of a sequence that is at least 90% identical to a sequence of any one of SEQ ID NOs: 1-8;
o. nucleotide 1 through nucleotide 34 of a sequence that is at least 90% identical to a sequence of SEQ ID NO: 9;
p. nucleotide 2 through nucleotide 34 of a sequence that is at least 90% identical to a sequence of SEQ ID NO: 9;
q. nucleotide 3 through nucleotide 34 of a sequence that is at least 90% identical to a sequence of SEQ ID NO: 9;
r. nucleotide 4 through nucleotide 34 of a sequence that is at least 90% identical to a sequence of SEQ ID NO: 9;
s. nucleotide 5 through nucleotide 34 of a sequence that is at least 90% identical to a sequence of SEQ ID NO: 9;
t. nucleotide 6 through nucleotide 34 of a sequence that is at least 90% identical to a sequence of SEQ ID NO: 9;
u. nucleotide 7 through nucleotide 34 of a sequence that is at least 90% identical to a sequence of SEQ ID NO: 9;
v. nucleotide 8 through nucleotide 34 of a sequence that is at least 90% identical to a sequence of SEQ ID NO: 9;
w. nucleotide 9 through nucleotide 34 of a sequence that is at least 90% identical to a sequence of SEQ ID NO: 9;
x. nucleotide 10 through nucleotide 34 of a sequence that is at least 90% identical to a sequence of SEQ ID NO: 9;
y. nucleotide 11 through nucleotide 34 of a sequence that is at least 90% identical to a sequence of SEQ ID NO: 9;
z. nucleotide 12 through nucleotide 34 of a sequence that is at least 90% identical to a sequence of SEQ ID NO: 9; or
aa, a sequence that is at least 90% identical to a sequence of SEQ ID NO: 10 or a portion thereof.

7. The composition of any one of claims 1 to 6, wherein the direct repeat comprises:
a. nucleotide 1 through nucleotide 36 of any one of SEQ ID NOs: 1-8;
b. nucleotide 2 through nucleotide 36 of any one of SEQ ID NOs: 1-8;
c. nucleotide 3 through nucleotide 36 of any one of SEQ ID NOs: 1-8;
d. nucleotide 4 through nucleotide 36 of any one of SEQ ID NOs: 1-8;
e. nucleotide 5 through nucleotide 36 of any one of SEQ ID NOs: 1-8;
f. nucleotide 6 through nucleotide 36 of any one of SEQ ID NOs: 1-8;
g. nucleotide 7 through nucleotide 36 of any one of SEQ ID NOs: 1-8;
h. nucleotide 8 through nucleotide 36 of any one of SEQ ID NOs: 1-8;
i. nucleotide 9 through nucleotide 36 of any one of SEQ ID NOs: 1-8;
j. nucleotide 10 through nucleotide 36 of any one of SEQ ID NOs: 1-8;
k. nucleotide 11 through nucleotide 36 of any one of SEQ ID NOs: 1-8;
l. nucleotide 12 through nucleotide 36 of any one of SEQ ID NOs: 1-8;
m. nucleotide 13 through nucleotide 36 of any one of SEQ ID NOs: 1-8;
n. nucleotide 14 through nucleotide 36 of any one of SEQ ID NOs: 1-8;
o. nucleotide 1 through nucleotide 34 of SEQ ID NO: 9;
p. nucleotide 2 through nucleotide 34 of SEQ ID NO: 9;
q. nucleotide 3 through nucleotide 34 of SEQ ID NO: 9;
r. nucleotide 4 through nucleotide 34 of SEQ ID NO: 9;
s. nucleotide 5 through nucleotide 34 of SEQ ID NO: 9;
t. nucleotide 6 through nucleotide 34 of SEQ ID NO: 9;
u. nucleotide 7 through nucleotide 34 of SEQ ID NO: 9;
v. nucleotide 8 through nucleotide 34 of SEQ ID NO: 9;
w. nucleotide 9 through nucleotide 34 of SEQ ID NO: 9;
x. nucleotide 10 through nucleotide 34 of SEQ ID NO: 9;
y. nucleotide 11 through nucleotide 34 of SEQ ID NO: 9;
z. nucleotide 12 through nucleotide 34 of SEQ ID NO: 9; or
aa. SEQ ID NO: 10 or a portion thereof.

8. The composition of any one of claims 1 to 5, wherein the direct repeat comprises:
a. nucleotide 1 through nucleotide 36 of a sequence that is at least 90% identical to a sequence of any one of SEQ ID NOs: 359-376;
b. nucleotide 2 through nucleotide 36 of a sequence that is at least 90% identical to a sequence of any one of SEQ ID NOs: 359-376;
c. nucleotide 3 through nucleotide 36 of a sequence that is at least 90% identical to a sequence of any one of SEQ ID NOs: 359-376;
d. nucleotide 4 through nucleotide 36 of a sequence that is at least 90% identical to a sequence of any one of SEQ ID NOs: 359-376;
e. nucleotide 5 through nucleotide 36 of a sequence that is at least 90% identical to a sequence of any one of SEQ ID NOs: 359-376;
f. nucleotide 6 through nucleotide 36 of a sequence that is at least 90% identical to a sequence of any one of SEQ ID NOs: 359-376;
g. nucleotide 7 through nucleotide 36 of a sequence that is at least 90% identical to a sequence of any one of SEQ ID NOs: 359-376;
h. nucleotide 8 through nucleotide 36 of a sequence that is at least 90% identical to a sequence of any one of SEQ ID NOs: 359-376;
i. nucleotide 9 through nucleotide 36 of a sequence that is at least 90% identical to a sequence of any one of SEQ ID NOs: 359-376;
j. nucleotide 10 through nucleotide 36 of a sequence that is at least 90% identical to a sequence of any one of SEQ ID NOs: 359-376;
k. nucleotide 11 through nucleotide 36 of a sequence that is at least 90% identical to a sequence of any one of SEQ ID NOs: 359-376;
l. nucleotide 12 through nucleotide 36 of a sequence that is at least 90% identical to a sequence of any one of SEQ ID NOs: 359-376;
m. nucleotide 13 through nucleotide 36 of a sequence that is at least 90% identical to a sequence of any one of SEQ ID NOs: 359-376;
n. nucleotide 14 through nucleotide 36 of a sequence that is at least 90% identical to a sequence of any one of SEQ ID NOs: 359-376; or
o. a sequence that is at least 90% identical to a sequence of SEQ ID NO: 377 or a portion thereof.

9. The composition of any one of claims 1 to 5 or 8, wherein the direct repeat comprises:
a. nucleotide 1 through nucleotide 36 of any one of SEQ ID NOs: 359-376;
b. nucleotide 2 through nucleotide 36 of any one of SEQ ID NOs: 359-376;
c. nucleotide 3 through nucleotide 36 of any one of SEQ ID NOs: 359-376;
d. nucleotide 4 through nucleotide 36 of any one of SEQ ID NOs: 359-376;
e. nucleotide 5 through nucleotide 36 of any one of SEQ ID NOs: 359-376;
f. nucleotide 6 through nucleotide 36 of any one of SEQ ID NOs: 359-376;
g. nucleotide 7 through nucleotide 36 of any one of SEQ ID NOs: 359-376;
h. nucleotide 8 through nucleotide 36 of any one of SEQ ID NOs: 359-376;
i. nucleotide 9 through nucleotide 36 of any one of SEQ ID NOs: 359-376;
j. nucleotide 10 through nucleotide 36 of any one of SEQ ID NOs: 359-376;
k. nucleotide 11 through nucleotide 36 of any one of SEQ ID NOs: 359-376;
l. nucleotide 12 through nucleotide 36 of any one of SEQ ID NOs: 359-376;
m. nucleotide 13 through nucleotide 36 of any one of SEQ ID NOs: 359-376;
n. nucleotide 14 through nucleotide 36 of any one of SEQ ID NOs: 359-376; or
o. SEQ ID NO: 377 or a portion thereof.

10. The composition of any one of claims 1 to 5, wherein the direct repeat comprises:
a. nucleotide 1 through nucleotide 36 of a sequence that is at least 90% identical to SEQ ID NO: 378;
b. nucleotide 2 through nucleotide 36 of a sequence that is at least 90% identical to SEQ ID NO: 378;
c. nucleotide 3 through nucleotide 36 of a sequence that is at least 90% identical to SEQ ID NO: 378;
d. nucleotide 4 through nucleotide 36 of a sequence that is at least 90% identical to SEQ ID NO: 378;
e. nucleotide 5 through nucleotide 36 of a sequence that is at least 90% identical to SEQ ID NO: 378;
f. nucleotide 6 through nucleotide 36 of a sequence that is at least 90% identical to SEQ ID NO: 378;
g. nucleotide 7 through nucleotide 36 of a sequence that is at least 90% identical to SEQ ID NO: 378;
h. nucleotide 8 through nucleotide 36 of a sequence that is at least 90% identical to SEQ ID NO: 378;
i. nucleotide 9 through nucleotide 36 of a sequence that is at least 90% identical to SEQ ID NO: 378;
j. nucleotide 10 through nucleotide 36 of a sequence that is at least 90% identical to SEQ ID NO: 378;
k. nucleotide 11 through nucleotide 36 of a sequence that is at least 90% identical to SEQ ID NO: 378;
l. nucleotide 12 through nucleotide 36 of a sequence that is at least 90% identical to SEQ ID NO: 378;
m. nucleotide 13 through nucleotide 36 of a sequence that is at least 90% identical to SEQ ID NO: 378;
n. nucleotide 14 through nucleotide 36 of a sequence that is at least 90% identical to SEQ ID NO: 378; or
o. a sequence that is at least 90% identical to a sequence of SEQ ID NO: 379 or SEQ ID NO: 380 or a portion thereof.

11. The composition of any one of claims 1 to 5 or 10, wherein the direct repeat comprises:
a. nucleotide 1 through nucleotide 36 of SEQ ID NO: 378;
b. nucleotide 2 through nucleotide 36 of SEQ ID NO: 378;
c. nucleotide 3 through nucleotide 36 of SEQ ID NO: 378;
d. nucleotide 4 through nucleotide 36 of SEQ ID NO: 378;
e. nucleotide 5 through nucleotide 36 of SEQ ID NO: 378;
f. nucleotide 6 through nucleotide 36 of SEQ ID NO: 378;
g. nucleotide 7 through nucleotide 36 of SEQ ID NO: 378;
h. nucleotide 8 through nucleotide 36 of SEQ ID NO: 378;
i. nucleotide 9 through nucleotide 36 of SEQ ID NO: 378;
j. nucleotide 10 through nucleotide 36 of SEQ ID NO: 378;
k. nucleotide 11 through nucleotide 36 of SEQ ID NO: 378;
l. nucleotide 12 through nucleotide 36 of SEQ ID NO: 378;
m. nucleotide 13 through nucleotide 36 of SEQ ID NO: 378;
n. nucleotide 14 through nucleotide 36 of SEQ ID NO: 378; or
o. SEQ ID NO: 379 or SEQ ID NO: 380 or a portion thereof.

12. The composition of any one of claims 1 to 5, wherein the direct repeat comprises:
a. nucleotide 1 through nucleotide 36 of a sequence that is at least 90% identical to a sequence of SEQ ID NO: 381 or SEQ ID NO: 382;
b. nucleotide 2 through nucleotide 36 of a sequence that is at least 90% identical to a sequence of SEQ ID NO: 381 or SEQ ID NO: 382;
c. nucleotide 3 through nucleotide 36 of a sequence that is at least 90% identical to a sequence of SEQ ID NO: 381 or SEQ ID NO: 382;
d. nucleotide 4 through nucleotide 36 of a sequence that is at least 90% identical to a sequence of SEQ ID NO: 381 or SEQ ID NO: 382;
e. nucleotide 5 through nucleotide 36 of a sequence that is at least 90% identical to a sequence of SEQ ID NO: 381 or SEQ ID NO: 382;
f. nucleotide 6 through nucleotide 36 of a sequence that is at least 90% identical to a sequence of SEQ ID NO: 381 or SEQ ID NO: 382;
g. nucleotide 7 through nucleotide 36 of a sequence that is at least 90% identical to a sequence of SEQ ID NO: 381 or SEQ ID NO: 382;
h. nucleotide 8 through nucleotide 36 of a sequence that is at least 90% identical to a sequence of SEQ ID NO: 381 or SEQ ID NO: 382;
i. nucleotide 9 through nucleotide 36 of a sequence that is at least 90% identical to a sequence of SEQ ID NO: 381 or SEQ ID NO: 382;
j. nucleotide 10 through nucleotide 36 of a sequence that is at least 90% identical to a sequence of SEQ ID NO: 381 or SEQ ID NO: 382;
k. nucleotide 11 through nucleotide 36 of a sequence that is at least 90% identical to a sequence of SEQ ID NO: 381 or SEQ ID NO: 382;
l. nucleotide 12 through nucleotide 36 of a sequence that is at least 90% identical to a sequence of SEQ ID NO: 381 or SEQ ID NO: 382;
m. nucleotide 13 through nucleotide 36 of a sequence that is at least 90% identical to a sequence of SEQ ID NO: 381 or SEQ ID NO: 382;
n. nucleotide 14 through nucleotide 36 of a sequence that is at least 90% identical to a sequence of SEQ ID NO: 381 or SEQ ID NO: 382;
o. nucleotide 15 through nucleotide 36 of a sequence that is at least 90% identical to a sequence of SEQ ID NO: 381 or SEQ ID NO: 382; or
p, a sequence that is at least 90% identical to a sequence of SEQ ID NO: 383 or a portion thereof.

13. The composition of any one of claims 1 to 5 or 12, wherein the direct repeat comprises:
a. nucleotide 1 through nucleotide 36 of SEQ ID NO: 381 or SEQ ID NO: 382;
b. nucleotide 2 through nucleotide 36 of SEQ ID NO: 381 or SEQ ID NO: 382;
c. nucleotide 3 through nucleotide 36 of SEQ ID NO: 381 or SEQ ID NO: 382;
d. nucleotide 4 through nucleotide 36 of SEQ ID NO: 381 or SEQ ID NO: 382;
e. nucleotide 5 through nucleotide 36 of SEQ ID NO: 381 or SEQ ID NO: 382;
f. nucleotide 6 through nucleotide 36 of SEQ ID NO: 381 or SEQ ID NO: 382;
g. nucleotide 7 through nucleotide 36 of SEQ ID NO: 381 or SEQ ID NO: 382;
h. nucleotide 8 through nucleotide 36 of SEQ ID NO: 381 or SEQ ID NO: 382;
i. nucleotide 9 through nucleotide 36 of SEQ ID NO: 381 or SEQ ID NO: 382;
j. nucleotide 10 through nucleotide 36 of SEQ ID NO: 381 or SEQ ID NO: 382;
k. nucleotide 11 through nucleotide 36 of SEQ ID NO: 381 or SEQ ID NO: 382;
l. nucleotide 12 through nucleotide 36 of SEQ ID NO: 381 or SEQ ID NO: 382;
m. nucleotide 13 through nucleotide 36 of SEQ ID NO: 381 or SEQ ID NO: 382;
n. nucleotide 14 through nucleotide 36 of SEQ ID NO: 381 or SEQ ID NO: 382;
o. nucleotide 15 through nucleotide 36 of SEQ ID NO: 381 or SEQ ID NO: 382; or
p. SEQ ID NO: 383 or a portion thereof.

14. The composition of any one of claims 1 to 13, wherein the spacer sequence is substantially complementary to the complement of a sequence of any one of SEQ ID NOs: 11-173.

15. The composition of claim 1, wherein the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTA-3′, 5′-CTTT-3′, 5′-CTTG-3′, or 5′-CTTC-3′.

16. The composition of claim 1 or 15, wherein the target sequence is immediately adjacent to the PAM sequence.

17. The composition of any one of claims 1 to 16, wherein the composition further comprises a Cas12i polypeptide.

18. The composition of claim 17, wherein the Cas12i polypeptide is:
a, a Cas12i2 polypeptide comprising a sequence that is at least 90% identical to the sequence of SEQ ID NO: 338, SEQ ID NO: 348, SEQ ID NO: 349, SEQ ID NO: 350, SEQ ID NO: 351, or SEQ ID NO: 352;
b, a Cas12i4 polypeptide comprising a sequence that is at least 90% identical to the sequence of SEQ ID NO: 354, SEQ ID NO: 355, or SEQ ID NO: 356;
c, a Cas12i1 polypeptide comprising a sequence that is at least 90% identical to the sequence of SEQ ID NO: 357; or
d, a Cas12i3 polypeptide comprising a sequence that is at least 90% identical to the sequence of SEQ ID NO: 358.

19. The composition of claim 18, wherein the Cas12i polypeptide is:
a, a Cas12i2 polypeptide comprising a sequence of SEQ ID NO: 338, SEQ ID NO: 348, SEQ ID NO: 349, SEQ ID NO: 350, SEQ ID NO: 351, or SEQ ID NO: 352;
b, a Cas12i4 polypeptide comprising a sequence of SEQ ID NO: 354, SEQ ID NO: 355, or SEQ ID NO: 356;
c, a Cas12i1 polypeptide comprising a sequence of SEQ ID NO: 357; or
d, a Cas12i3 polypeptide comprising a sequence of SEQ ID NO: 358.

20. The composition of any one of claims 17 to 19, wherein the RNA guide and the Cas2i polypeptide form a ribonucleoprotein complex.

21. The composition of claim 20, wherein the ribonucleoprotein complex binds a target nucleic acid.

22. The composition of claim 20 or 21, wherein the composition is present within a cell.

23. The composition of any one of claims 17 to 22, wherein the RNA guide and the Cas2i polypeptide are encoded in a vector, e.g., expression vector.

24. The composition of claim 23, wherein the RNA guide and the Cas12i polypeptide are encoded in a single vector or the RNA guide is encoded in a first vector and the Cas12i polypeptide is encoded in a second vector.

25. An RNA guide comprising (i) a spacer sequence that is substantially complementary to a target sequence within a TRAC gene and (ii) a direct repeat sequence.

26. The RNA guide of claim 25, wherein the target sequence is within exon 1, exon 2, exon 3, or exon 4 of the TRAC gene.

27. The RNA guide of claim 25 or 26, wherein the TRAC gene comprises the sequence of SEQ ID NO: 339, the reverse complement of SEQ ID NO: 339, a variant of the sequence of SEQ ID NO: 339, or the reverse complement of SEQ ID NO: 339.

28. The RNA guide of any one of claims 25 to 27, wherein the spacer sequence comprises:
a. nucleotide 1 through nucleotide 16 of a sequence that is at least 90% identical to a sequence of any one of SEQ ID NOs: 174-336;
b. nucleotide 1 through nucleotide 17 of a sequence that is at least 90% identical to a sequence of any one of SEQ ID NOs: 174-336;
c. nucleotide 1 through nucleotide 18 of a sequence that is at least 90% identical to a sequence of any one of SEQ ID NOs: 174-336;
d. nucleotide 1 through nucleotide 19 of a sequence that is at least 90% identical to a sequence of any one of SEQ ID NOs: 174-336;
e. nucleotide 1 through nucleotide 20 of a sequence that is at least 90% identical to a sequence of any one of SEQ ID NOs: 174-336;
f. nucleotide 1 through nucleotide 21 of a sequence that is at least 90% identical to a sequence of any one of SEQ ID NOs: 174-336;
g. nucleotide 1 through nucleotide 22 of a sequence that is at least 90% identical to a sequence of any one of SEQ ID NOs: 174-336;
h. nucleotide 1 through nucleotide 23 of a sequence that is at least 90% identical to a sequence of any one of SEQ ID NOs: 174-336;
i. nucleotide 1 through nucleotide 24 of a sequence that is at least 90% identical to a sequence of any one of SEQ ID NOs: 174-336;
j. nucleotide 1 through nucleotide 25 of a sequence that is at least 90% identical to a sequence of any one of SEQ ID NOs: 174-275 and 277-336;
k. nucleotide 1 through nucleotide 26 of a sequence that is at least 90% identical to a sequence of any one of SEQ ID NOs: 174-275 and 277-336;
l. nucleotide 1 through nucleotide 27 of a sequence that is at least 90% identical to a sequence of any one of SEQ ID NOs: 174-275 and 277-336;
m. nucleotide 1 through nucleotide 28 of a sequence that is at least 90% identical to a sequence of any one of SEQ ID NOs: 174-275 and 277-336;
n. nucleotide 1 through nucleotide 29 of a sequence that is at least 90% identical to a sequence of any one of SEQ ID NOs: 174-275 and 277-336; or
o. nucleotide 1 through nucleotide 30 of a sequence that is at least 90% identical to a sequence of any one of SEQ ID NOs: 174-275 and 277-336.

29. The RNA guide of any one of claims 25 to 28, wherein the spacer sequence comprises:
a. nucleotide 1 through nucleotide 16 of any one of SEQ ID NOs: 174-336;
b. nucleotide 1 through nucleotide 17 of any one of SEQ ID NOs: 174-336;
c. nucleotide 1 through nucleotide 18 of any one of SEQ ID NOs: 174-336;
d. nucleotide 1 through nucleotide 19 of any one of SEQ ID NOs: 174-336;
e. nucleotide 1 through nucleotide 20 of any one of SEQ ID NOs: 174-336;
f. nucleotide 1 through nucleotide 21 of any one of SEQ ID NOs: 174-336;
g. nucleotide 1 through nucleotide 22 of any one of SEQ ID NOs: 174-336;
h. nucleotide 1 through nucleotide 23 of any one of SEQ ID NOs: 174-336;
i. nucleotide 1 through nucleotide 24 of any one of SEQ ID NOs: 174-336;
j. nucleotide 1 through nucleotide 25 of any one of SEQ ID NOs: 174-275 and 277-336;
k. nucleotide 1 through nucleotide 26 of any one of SEQ ID NOs: 174-275 and 277-336;
l. nucleotide 1 through nucleotide 27 of any one of SEQ ID NOs: 174-275 and 277-336;
m. nucleotide 1 through nucleotide 28 of any one of SEQ ID NOs: 174-275 and 277-336;
n. nucleotide 1 through nucleotide 29 of any one of SEQ ID NOs: 174-275 and 277-336; or
o. nucleotide 1 through nucleotide 30 of any one of SEQ ID NOs: 174-275 and 277-336.

30. The RNA guide of any one of claims 25 to 29, wherein the direct repeat comprises:
a. nucleotide 1 through nucleotide 36 of a sequence that is at least 90% identical to a sequence of any one of SEQ ID NOs: 1-8;
b. nucleotide 2 through nucleotide 36 of a sequence that is at least 90% identical to a sequence of any one of SEQ ID NOs: 1-8;
c. nucleotide 3 through nucleotide 36 of a sequence that is at least 90% identical to a sequence of any one of SEQ ID NOs: 1-8;
d. nucleotide 4 through nucleotide 36 of a sequence that is at least 90% identical to a sequence of any one of SEQ ID NOs: 1-8;
e. nucleotide 5 through nucleotide 36 of a sequence that is at least 90% identical to a sequence of any one of SEQ ID NOs: 1-8;
f. nucleotide 6 through nucleotide 36 of a sequence that is at least 90% identical to a sequence of any one of SEQ ID NOs: 1-8;
g. nucleotide 7 through nucleotide 36 of a sequence that is at least 90% identical to a sequence of any one of SEQ ID NOs: 1-8;
h. nucleotide 8 through nucleotide 36 of a sequence that is at least 90% identical to a sequence of any one of SEQ ID NOs: 1-8;
i. nucleotide 9 through nucleotide 36 of a sequence that is at least 90% identical to a sequence of any one of SEQ ID NOs: 1-8;
j. nucleotide 10 through nucleotide 36 of a sequence that is at least 90% identical to a sequence of any one of SEQ ID NOs: 1-8;
k. nucleotide 11 through nucleotide 36 of a sequence that is at least 90% identical to a sequence of any one of SEQ ID NOs: 1-8;
l. nucleotide 12 through nucleotide 36 of a sequence that is at least 90% identical to a sequence of any one of SEQ ID NOs: 1-8;
m. nucleotide 13 through nucleotide 36 of a sequence that is at least 90% identical to a sequence of any one of SEQ ID NOs: 1-8;
n. nucleotide 14 through nucleotide 36 of a sequence that is at least 90% identical to a sequence of any one of SEQ ID NOs: 1-8;
o. nucleotide 1 through nucleotide 34 of a sequence that is at least 90% identical to a sequence of SEQ ID NO: 9;
p. nucleotide 2 through nucleotide 34 of a sequence that is at least 90% identical to a sequence of SEQ ID NO: 9;
q. nucleotide 3 through nucleotide 34 of a sequence that is at least 90% identical to a sequence of SEQ ID NO: 9;
r. nucleotide 4 through nucleotide 34 of a sequence that is at least 90% identical to a sequence of SEQ ID NO: 9;
s. nucleotide 5 through nucleotide 34 of a sequence that is at least 90% identical to a sequence of SEQ ID NO: 9;
t. nucleotide 6 through nucleotide 34 of a sequence that is at least 90% identical to a sequence of SEQ ID NO: 9;
u. nucleotide 7 through nucleotide 34 of a sequence that is at least 90% identical to a sequence of SEQ ID NO: 9;
v. nucleotide 8 through nucleotide 34 of a sequence that is at least 90% identical to a sequence of SEQ ID NO: 9;
w. nucleotide 9 through nucleotide 34 of a sequence that is at least 90% identical to a sequence of SEQ ID NO: 9;
x. nucleotide 10 through nucleotide 34 of a sequence that is at least 90% identical to a sequence of SEQ ID NO: 9;
y. nucleotide 11 through nucleotide 34 of a sequence that is at least 90% identical to a sequence of SEQ ID NO: 9;
z. nucleotide 12 through nucleotide 34 of a sequence that is at least 90% identical to a sequence of SEQ ID NO: 9; or
aa, a sequence that is at least 90% identical to a sequence of SEQ ID NO: 10 or a portion thereof.

31. The RNA guide of any one of claims 25 to 30, wherein the direct repeat comprises:
a. nucleotide 1 through nucleotide 36 of any one of SEQ ID NOs: 1-8;
b. nucleotide 2 through nucleotide 36 of any one of SEQ ID NOs: 1-8;
c. nucleotide 3 through nucleotide 36 of any one of SEQ ID NOs: 1-8;
d. nucleotide 4 through nucleotide 36 of any one of SEQ ID NOs: 1-8;
e. nucleotide 5 through nucleotide 36 of any one of SEQ ID NOs: 1-8;
f. nucleotide 6 through nucleotide 36 of any one of SEQ ID NOs: 1-8;
g. nucleotide 7 through nucleotide 36 of any one of SEQ ID NOs: 1-8;
h. nucleotide 8 through nucleotide 36 of any one of SEQ ID NOs: 1-8;
i. nucleotide 9 through nucleotide 36 of any one of SEQ ID NOs: 1-8;
j. nucleotide 10 through nucleotide 36 of any one of SEQ ID NOs: 1-8;
k. nucleotide 11 through nucleotide 36 of any one of SEQ ID NOs: 1-8;
l. nucleotide 12 through nucleotide 36 of any one of SEQ ID NOs: 1-8;
m. nucleotide 13 through nucleotide 36 of any one of SEQ ID NOs: 1-8;
n. nucleotide 14 through nucleotide 36 of any one of SEQ ID NOs: 1-8;
o. nucleotide 1 through nucleotide 34 of SEQ ID NO: 9;
p. nucleotide 2 through nucleotide 34 of SEQ ID NO: 9;
q. nucleotide 3 through nucleotide 34 of SEQ ID NO: 9;
r. nucleotide 4 through nucleotide 34 of SEQ ID NO: 9;
s. nucleotide 5 through nucleotide 34 of SEQ ID NO: 9;
t. nucleotide 6 through nucleotide 34 of SEQ ID NO: 9;
u. nucleotide 7 through nucleotide 34 of SEQ ID NO: 9;
v. nucleotide 8 through nucleotide 34 of SEQ ID NO: 9;
w. nucleotide 9 through nucleotide 34 of SEQ ID NO: 9;
x. nucleotide 10 through nucleotide 34 of SEQ ID NO: 9;
y. nucleotide 11 through nucleotide 34 of SEQ ID NO: 9;
z. nucleotide 12 through nucleotide 34 of SEQ ID NO: 9; or
aa. SEQ ID NO: 10 or a portion thereof.

32. The RNA guide of any one of claims 25 to 31, wherein the direct repeat comprises:
a. nucleotide 1 through nucleotide 36 of a sequence that is at least 90% identical to a sequence of any one of SEQ ID NOs: 359-376;
b. nucleotide 2 through nucleotide 36 of a sequence that is at least 90% identical to a sequence of any one of SEQ ID NOs: 359-376;
c. nucleotide 3 through nucleotide 36 of a sequence that is at least 90% identical to a sequence of any one of SEQ ID NOs: 359-376;
d. nucleotide 4 through nucleotide 36 of a sequence that is at least 90% identical to a sequence of any one of SEQ ID NOs: 359-376;
e. nucleotide 5 through nucleotide 36 of a sequence that is at least 90% identical to a sequence of any one of SEQ ID NOs: 359-376;
f. nucleotide 6 through nucleotide 36 of a sequence that is at least 90% identical to a sequence of any one of SEQ ID NOs: 359-376;
g. nucleotide 7 through nucleotide 36 of a sequence that is at least 90% identical to a sequence of any one of SEQ ID NOs: 359-376;
h. nucleotide 8 through nucleotide 36 of a sequence that is at least 90% identical to a sequence of any one of SEQ ID NOs: 359-376;
i. nucleotide 9 through nucleotide 36 of a sequence that is at least 90% identical to a sequence of any one of SEQ ID NOs: 359-376;
j. nucleotide 10 through nucleotide 36 of a sequence that is at least 90% identical to a sequence of any one of SEQ ID NOs: 359-376;
k. nucleotide 11 through nucleotide 36 of a sequence that is at least 90% identical to a sequence of any one of SEQ ID NOs: 359-376;
l. nucleotide 12 through nucleotide 36 of a sequence that is at least 90% identical to a sequence of any one of SEQ ID NOs: 359-376;
m. nucleotide 13 through nucleotide 36 of a sequence that is at least 90% identical to a sequence of any one of SEQ ID NOs: 359-376;
n. nucleotide 14 through nucleotide 36 of a sequence that is at least 90% identical to a sequence of any one of SEQ ID NOs: 359-376; or
o. a sequence that is at least 90% identical to a sequence of SEQ ID NO: 377 or a portion thereof.

33. The RNA guide of any one of claims 25 to 29 or 32, wherein the direct repeat comprises:
a. nucleotide 1 through nucleotide 36 of any one of SEQ ID NOs: 359-376;
b. nucleotide 2 through nucleotide 36 of any one of SEQ ID NOs: 359-376;
c. nucleotide 3 through nucleotide 36 of any one of SEQ ID NOs: 359-376;
d. nucleotide 4 through nucleotide 36 of any one of SEQ ID NOs: 359-376;
e. nucleotide 5 through nucleotide 36 of any one of SEQ ID NOs: 359-376;
f. nucleotide 6 through nucleotide 36 of any one of SEQ ID NOs: 359-376;
g. nucleotide 7 through nucleotide 36 of any one of SEQ ID NOs: 359-376;
h. nucleotide 8 through nucleotide 36 of any one of SEQ ID NOs: 359-376;
i. nucleotide 9 through nucleotide 36 of any one of SEQ ID NOs: 359-376;
j. nucleotide 10 through nucleotide 36 of any one of SEQ ID NOs: 359-376;
k. nucleotide 11 through nucleotide 36 of any one of SEQ ID NOs: 359-376;
l. nucleotide 12 through nucleotide 36 of any one of SEQ ID NOs: 359-376;
m. nucleotide 13 through nucleotide 36 of any one of SEQ ID NOs: 359-376;
n. nucleotide 14 through nucleotide 36 of any one of SEQ ID NOs: 359-376; or
o. SEQ ID NO: 377 or a portion thereof.

34. The RNA guide of any one of claims 25 to 29, wherein the direct repeat comprises:
a. nucleotide 1 through nucleotide 36 of a sequence that is at least 90% identical to SEQ ID NO: 378;
b. nucleotide 2 through nucleotide 36 of a sequence that is at least 90% identical to SEQ ID NO: 378;
c. nucleotide 3 through nucleotide 36 of a sequence that is at least 90% identical to SEQ ID NO: 378;
d. nucleotide 4 through nucleotide 36 of a sequence that is at least 90% identical to SEQ ID NO: 378;
e. nucleotide 5 through nucleotide 36 of a sequence that is at least 90% identical to SEQ ID NO: 378;
f. nucleotide 6 through nucleotide 36 of a sequence that is at least 90% identical to SEQ ID NO: 378;
g. nucleotide 7 through nucleotide 36 of a sequence that is at least 90% identical to SEQ ID NO: 378;
h. nucleotide 8 through nucleotide 36 of a sequence that is at least 90% identical to SEQ ID NO: 378;
i. nucleotide 9 through nucleotide 36 of a sequence that is at least 90% identical to SEQ ID NO: 378;
j. nucleotide 10 through nucleotide 36 of a sequence that is at least 90% identical to SEQ ID NO: 378;
k. nucleotide 11 through nucleotide 36 of a sequence that is at least 90% identical to SEQ ID NO: 378;
l. nucleotide 12 through nucleotide 36 of a sequence that is at least 90% identical to SEQ ID NO: 378;
m. nucleotide 13 through nucleotide 36 of a sequence that is at least 90% identical to SEQ ID NO: 378;
n. nucleotide 14 through nucleotide 36 of a sequence that is at least 90% identical to SEQ ID NO: 378; or
o. a sequence that is at least 90% identical to a sequence of SEQ ID NO: 379 or SEQ ID NO: 380 or a portion thereof.

35. The RNA guide of any one of claims 25 to 29 or 34, wherein the direct repeat comprises:
a. nucleotide 1 through nucleotide 36 of SEQ ID NO: 378;
b. nucleotide 2 through nucleotide 36 of SEQ ID NO: 378;
c. nucleotide 3 through nucleotide 36 of SEQ ID NO: 378;
d. nucleotide 4 through nucleotide 36 of SEQ ID NO: 378;
e. nucleotide 5 through nucleotide 36 of SEQ ID NO: 378;
f. nucleotide 6 through nucleotide 36 of SEQ ID NO: 378;
g. nucleotide 7 through nucleotide 36 of SEQ ID NO: 378;
h. nucleotide 8 through nucleotide 36 of SEQ ID NO: 378;
i. nucleotide 9 through nucleotide 36 of SEQ ID NO: 378;
j. nucleotide 10 through nucleotide 36 of SEQ ID NO: 378;
k. nucleotide 11 through nucleotide 36 of SEQ ID NO: 378;
l. nucleotide 12 through nucleotide 36 of SEQ ID NO: 378;
m. nucleotide 13 through nucleotide 36 of SEQ ID NO: 378;
n. nucleotide 14 through nucleotide 36 of SEQ ID NO: 378; or
o. SEQ ID NO: 379 or SEQ ID NO: 380 or a portion thereof.

36. The RNA guide of any one of claims 25 to 29, wherein the direct repeat comprises:
a. nucleotide 1 through nucleotide 36 of a sequence that is at least 90% identical to a sequence of SEQ ID NO: 381 or SEQ ID NO: 382;
b. nucleotide 2 through nucleotide 36 of a sequence that is at least 90% identical to a sequence of SEQ ID NO: 381 or SEQ ID NO: 382;
c. nucleotide 3 through nucleotide 36 of a sequence that is at least 90% identical to a sequence of SEQ ID NO: 381 or SEQ ID NO: 382;
d. nucleotide 4 through nucleotide 36 of a sequence that is at least 90% identical to a sequence of SEQ ID NO: 381 or SEQ ID NO: 382;
e. nucleotide 5 through nucleotide 36 of a sequence that is at least 90% identical to a sequence of SEQ ID NO: 381 or SEQ ID NO: 382;
f. nucleotide 6 through nucleotide 36 of a sequence that is at least 90% identical to a sequence of SEQ ID NO: 381 or SEQ ID NO: 382;
g. nucleotide 7 through nucleotide 36 of a sequence that is at least 90% identical to a sequence of SEQ ID NO: 381 or SEQ ID NO: 382;
h. nucleotide 8 through nucleotide 36 of a sequence that is at least 90% identical to a sequence of SEQ ID NO: 381 or SEQ ID NO: 382;
i. nucleotide 9 through nucleotide 36 of a sequence that is at least 90% identical to a sequence of SEQ ID NO: 381 or SEQ ID NO: 382;
j. nucleotide 10 through nucleotide 36 of a sequence that is at least 90% identical to a sequence of SEQ ID NO: 381 or SEQ ID NO: 382;
k. nucleotide 11 through nucleotide 36 of a sequence that is at least 90% identical to a sequence of SEQ ID NO: 381 or SEQ ID NO: 382;
l. nucleotide 12 through nucleotide 36 of a sequence that is at least 90% identical to a sequence of SEQ ID NO: 381 or SEQ ID NO: 382;
m. nucleotide 13 through nucleotide 36 of a sequence that is at least 90% identical to a sequence of SEQ ID NO: 381 or SEQ ID NO: 382;
n. nucleotide 14 through nucleotide 36 of a sequence that is at least 90% identical to a sequence of SEQ ID NO: 381 or SEQ ID NO: 382;
o. nucleotide 15 through nucleotide 36 of a sequence that is at least 90% identical to a sequence of SEQ ID NO: 381 or SEQ ID NO: 382; or
p, a sequence that is at least 90% identical to a sequence of SEQ ID NO: 383 or a portion thereof.

37. The RNA guide of any one of claims 25 to 29 or 36, wherein the direct repeat comprises:
a. nucleotide 1 through nucleotide 36 of SEQ ID NO: 381 or SEQ ID NO: 382;
b. nucleotide 2 through nucleotide 36 of SEQ ID NO: 381 or SEQ ID NO: 382;
c. nucleotide 3 through nucleotide 36 of SEQ ID NO: 381 or SEQ ID NO: 382;
d. nucleotide 4 through nucleotide 36 of SEQ ID NO: 381 or SEQ ID NO: 382;
e. nucleotide 5 through nucleotide 36 of SEQ ID NO: 381 or SEQ ID NO: 382;
f. nucleotide 6 through nucleotide 36 of SEQ ID NO: 381 or SEQ ID NO: 382;
g. nucleotide 7 through nucleotide 36 of SEQ ID NO: 381 or SEQ ID NO: 382;
h. nucleotide 8 through nucleotide 36 of SEQ ID NO: 381 or SEQ ID NO: 382;
i. nucleotide 9 through nucleotide 36 of SEQ ID NO: 381 or SEQ ID NO: 382;
j. nucleotide 10 through nucleotide 36 of SEQ ID NO: 381 or SEQ ID NO: 382;
k. nucleotide 11 through nucleotide 36 of SEQ ID NO: 381 or SEQ ID NO: 382;
l. nucleotide 12 through nucleotide 36 of SEQ ID NO: 381 or SEQ ID NO: 382;
m. nucleotide 13 through nucleotide 36 of SEQ ID NO: 381 or SEQ ID NO: 382;
n. nucleotide 14 through nucleotide 36 of SEQ ID NO: 381 or SEQ ID NO: 382;
o. nucleotide 15 through nucleotide 36 of SEQ ID NO: 381 or SEQ ID NO: 382; or
p. SEQ ID NO: 383 or a portion thereof.

38. The RNA guide of any one of claims 25 to 37, wherein the spacer sequence is substantially complementary to the complement of a sequence of any one of SEQ ID NOs: 11-173.

39. The RNA guide of any one of claims 25 to 38, wherein the target sequence is adjacent to a protospacer adjacent motif (PAM) comprising the sequence 5′-NTTN-3′, wherein N is any nucleotide.

40. The RNA guide of claim 39, wherein the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

41. The RNA guide of claim 39 or 40, wherein the target sequence is immediately adjacent to the PAM sequence.

42. A nucleic acid encoding an RNA guide of any one of claims 25 to 41.

43. A vector comprising the nucleic acid of claim 42.

44. A vector system comprising one or more vectors encoding (i) the RNA guide as defined in any of claims 1 to 41 and (ii) a Cas12i polypeptide, optionally wherein the vector system comprises a first vector encoding the RNA guide and a second vector encoding the Cas12i polypeptide.

45. A cell comprising the composition of any one of claims 1 to 24, the RNA guide of any one of claims 25 to 41, the nucleic acid of claim 42, the vector of claim 43, or the vector system of claim 44.

46. The cell of claim 45, wherein the cell is a eukaryotic cell, an animal cell, a mammalian cell, a human cell, a primary cell, a cell line, a stem cell, or a T cell.

47. A kit comprising the composition of any one of claims 1 to 24, the RNA guide of any one of claims to 41, the nucleic acid of claim 42, the vector of claim 43, or the vector system of claim 44.

48. A method of editing a TRAC sequence, the method comprising contacting a TRAC sequence with a composition of any one of claims 1 to 24 or an RNA guide of any one of claims 25 to 41.

49. The method of claim 48, wherein the TRAC sequence is in a cell.

50. The method of claim 48 or 49, wherein the composition or the RNA guide induces a deletion in the TRAC sequence.

51. The method of claim 50, wherein the deletion is adjacent to a 5′-NTTN-3′ sequence, wherein N is any nucleotide.

52. The method of claim 50 or 51, wherein the deletion is downstream of the 5′-NTTN-3′ sequence.

53. The method of any one of claims 50 to 52, wherein the deletion is up to about 50 nucleotides in length.

54. The method of any one of claims 50 to 53, wherein the deletion is up to about 40 nucleotides in length.

55. The method of any one of claims 50 to 54, wherein the deletion is from about 4 nucleotides to 40 nucleotides in length.

56. The method of any one of claims 50 to 55, wherein the deletion is from about 4 nucleotides to 25 nucleotides in length.

57. The method of any one of claims 50 to 56, wherein the deletion is from about 10 nucleotides to 25 nucleotides in length.

58. The method of any one of claims 50 to 57, wherein the deletion is from about 10 nucleotides to 15 nucleotides in length.

59. The method of any one of claims 50 to 58, wherein the deletion starts within about 5 nucleotides to about 15 nucleotides of the 5′-NTTN-3′ sequence.

60. The method of any one of claims 50 to 59, wherein the deletion starts within about 5 nucleotides to about 10 nucleotides of the 5′-NTTN-3′ sequence.

61. The method of any one of claims 50 to 60, wherein the deletion starts within about 10 nucleotides to about 15 nucleotides of the 5′-NTTN-3′ sequence.

62. The method of any one of claims 50 to 61, wherein the deletion starts within about 5 nucleotides to about 15 nucleotides downstream of the 5′-NTTN-3′ sequence.

63. The method of any one of claims 50 to 62, wherein the deletion starts within about 5 nucleotides to about 10 nucleotides downstream of the 5′-NTTN-3′ sequence.

64. The method of any one of claims 50 to 63, wherein the deletion starts within about 10 nucleotides to about 15 nucleotides downstream of the 5′-NTTN-3′ sequence.

65. The method of any one of claims 50 to 64, wherein the deletion ends within about 20 nucleotides to about 30 nucleotides of the 5′-NTTN-3′ sequence.

66. The method of any one of claims 50 to 65, wherein the deletion ends within about 20 nucleotides to about 25 nucleotides of the 5′-NTTN-3′ sequence.

67. The method of any one of claims 50 to 66, wherein the deletion ends within about 25 nucleotides to about 30 nucleotides of the 5′-NTTN-3′ sequence.

68. The method of any one of claims 50 to 67, wherein the deletion ends within about 20 nucleotides to about 30 nucleotides downstream of the 5′-NTTN-3′ sequence.

69. The method of any one of claims 50 to 68, wherein the deletion ends within about 20 nucleotides to about 25 nucleotides downstream of the 5′-NTTN-3′ sequence.

70. The method of any one of claims 50 to 69, wherein the deletion ends within about 25 nucleotides to about 30 nucleotides downstream of the 5′-NTTN-3′ sequence.

71. The method of any one of claims 50 to 70, wherein the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

72. The method of any one of claims 50 to 71, wherein the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

73. The method of any one of claims 50 to 72, wherein the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

74. The method of any one of claims 50 to 73, wherein the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

75. The method of any one of claims 50 to 74, wherein the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

76. The method of any one of claims 50 to 75, wherein the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

77. The method of any one of claims 50 to 76, wherein the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

78. The method of any one of claims 50 to 77, wherein the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

79. The method of any one of claims 50 to 78, wherein the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

80. The method of any one of claims 50 to 79, wherein the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

81. The method of any one of claims 50 to 80, wherein the deletion overlaps with a mutation in the TRAC sequence.

82. The method of any one of claims 50 to 81, wherein the deletion overlaps with an insertion in the TRAC sequence.

83. The method of any one of claims 50 to 82, wherein the deletion removes a repeat expansion of the TRAC sequence or a portion thereof.

84. The method of any one of claims 50 to 83, wherein the deletion disrupts one or both alleles of the TRAC sequence.

85. The composition, RNA guide, nucleic acid, vector, cell, kit or method of any one of the previous claims, wherein the RNA guide does not consist of the sequence of:
(SEQ ID NO: 344)
AGAAAUCCGUCUUUCAUUGACGGAAGAGCAACAGUGCUGUGGC;
(SEQ ID NO: 345)
AGAAAUCCGUCUUUCAUUGACGGAACAACAGCAUUAUUCCAGA;
(SEQ ID NO: 346)
AGAAAUCCGUCUUUCAUUGACGGGAAACAGGUAAGACAGGGGU;
or
(SEQ ID NO: 347)
AGAAAUCCGUCUUUCAUUGACGGCAGGAGGAGGAUUCGGAACC.

86. The composition, RNA guide, nucleic acid, vector, cell, kit or method of any one of the previous claims, wherein the RNA guide comprises the sequence of any one of SEQ ID NOs: 385-397.
